(19) United States
(12) Patent Application Publication
(10) Pub. No.: US 2026/0049077 A1
(43) Pub. Date: Feb. 19, 2026

(54) Neurotrophic Receptor Tyrosine Kinase (NTRK) Inhibitors and Methods Using Same

(71) Applicant: Baylor College of Medicine, Houston, TX (US)

(72) Inventors: Martin M. Matzuk, Houston, TX (US); Yong Wang, Houston, TX (US); Diana Monsivais, Houston, TX (US); Ravikumar Jimmindi, Houston, TX (US); Feng Li, Houston, TX (US); Srinivas Chamakuri, Houston, TX (US); Mingxing Teng, Houston, TX (US); Ramakrishna Kommagani, Houston, TX (US); Zhi Tan, Houston, TX (US)

(21) Appl. No.: 19/101,764

(22) PCT Filed: Jul. 27, 2023
(86) PCT No.: PCT/US2023/071125

§ 371 Date: Feb. 6, 2025

Related U.S. Application Data
(60) Provisional application No. 63/393,557, filed on Jul. 29, 2022.

(51) Int. Cl. C07D 471/04; A61K 31/17; A61K 31/404; A61K 31/4184; A61K 31/437; A61K 31/4409; A61K 31/4439; A61K 31/444; A61K 31/496; A61K 31/505; A61K 31/519; A61K 31/529; A61K 31/538; A61K 31/553; A61P 15/00; A61P 29/00; C07C 275/42; C07D 209/08; C07D 213/40; C07D 235/18; C07D 239/26; C07D 401/12; C07D 413/14; C07D 519/00

(52) U.S. Cl. CPC C07D 471/04; A61K 31/17; A61K 31/404; A61K 31/4184; A61K 31/437; A61K 31/4409; A61K 31/4439; A61K 31/444; A61K 31/496; A61K 31/505; A61K 31/519; A61K 31/529; A61K 31/538; A61K 31/553; A61P 15/00; A61P 29/00; C07C 275/42; C07D 209/08; C07D 213/40; C07D 235/18; C07D 239/26; C07D 401/12; C07D 413/14; C07D 519/00

(57) ABSTRACT

The present disclosure relates to compounds which inhibit one or more members of the neurotrophic receptor tyrosine kinase (NTRK) family (i.e., NTRK1, NTRK2, and/or NTRK3), and pharmaceutical compositions thereof. In another aspect, the present disclosure provides a method of treating, preventing, and/or ameliorating at least one disease and/or disorder selected from the group consisting of endometriosis, cancer, and pain in a subject in need thereof, the method comprising administering to the subject a therapeutically effective amount of a compound of the present disclosure and/or a pharmaceutical composition of the present disclosure.

### CROSS-REFERENCE TO RELATED APPLICATIONS

This application claims priority under 35 U.S.C. § 119(e) to U.S. Provisional Patent Application No. 63/393,557, filed Jul. 29, 2022, which is incorporated herein by reference in its entirety.

### BACKGROUND

The human genome encodes 538 protein kinases, most of which are functionally important and are involved in the pathogenesis of many diseases. Small molecule inhibition of the enzymatic activities of kinases is an attractive and tractable approach for the treatment of disease. Currently, there are approximately 73 kinase inhibitors with Food and Drug Administration (FDA) approval for the treatment of various diseases, indicating that kinase inhibition is a validated therapeutic approach with an opportunity for substantial development and advancement.

Endometriosis is a debilitating inflammatory disease that is associated with dysmenorrhea, dyspareunia, and menorrhagia, causes infertility, and results in increased incidence of endometrioid and clear-cell ovarian cancer. In women with endometriosis, endometrial tissue enters the peritoneal cavity via retrograde menstruation, leading to the attachment and persistent growth of endometrial tissue outside of the uterine cavity. The disease affects approximately 190 million (˜10%) of women ages 15-49 worldwide.

Women with endometriosis will typically endure the painful symptoms of the disease for about 10 years before receiving a conclusive diagnosis. Further, even after diagnosis, available treatments for endometriosis are limited and often ineffective long-term. For example, surgical excision of endometriotic implants is not completely curative, as lesions, and associated pain will typically recur. The debilitating pain and host of related side effects of endometriosis prevent women from experiencing productive and vibrant lives during the peak years of their lives.

Thus, there is an urgent need in the art for compositions for the treatment, prevention, and/or amelioration of endometriosis, and methods of use thereof. The present disclosure addresses this need.

### BRIEF SUMMARY

The present disclosure provides, in one aspect, a compound of formula (I), or a salt, solvate, isotopologue, prodrug, stereoisomer, or tautomer thereof, or any mixtures thereof, wherein T, L¹, L², R⁴, and R⁵ are defined elsewhere herein:

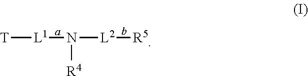

In another aspect, the present disclosure provides a compound of formula (Ia), or a salt, solvate, isotopologue, prodrug, stereoisomer, or tautomer thereof, or any mixtures thereof, wherein L¹, L², R¹, R², R^{3a}, R^{3b}, R^{3c}, R⁴, and R⁵ are defined elsewhere herein:

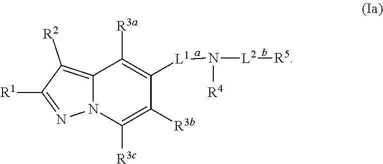

In another aspect, the present disclosure provides a compound of formula (Ib), or a salt, solvate, isotopologue, prodrug, stereoisomer, or tautomer thereof, or any mixtures thereof, wherein L¹, L², R¹, R², R^{3a}, R^{3b}, R^{3c}, R⁴, and R⁵ are defined elsewhere herein:

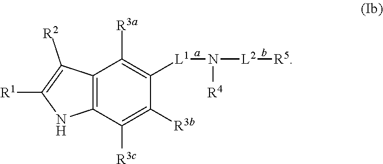

In another aspect, the present disclosure provides a compound of formula (Ic), or a salt, solvate, isotopologue, prodrug, stereoisomer, or tautomer thereof, or any mixtures thereof, wherein L¹, L², R¹, R^{3a}, R^{3b}, R^{3c}, R⁴, and R⁵ are defined elsewhere herein:

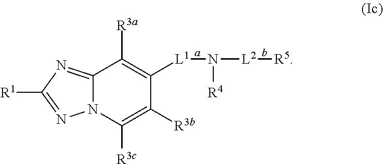

In another aspect, the present disclosure provides a compound of formula (Id), or a salt, solvate, isotopologue, prodrug, stereoisomer, or tautomer thereof, or any mixtures thereof, wherein L¹, L², R¹, R², R^{3a}, R^{3b}, R^{3c}, R⁴, and R⁵ are defined elsewhere herein:

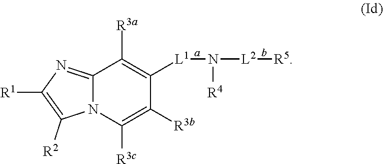

In another aspect, the present disclosure provides a compound of formula (Ie), or a salt, solvate, isotopologue, prodrug, stereoisomer, or tautomer thereof, or any mixtures thereof, wherein L¹, L², R^{3a}, R^{3b}, R^{3c}, R^{3d}, R⁴, and R⁵ are defined elsewhere herein:

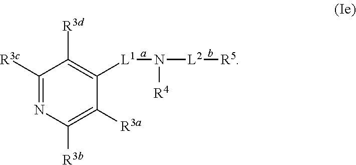

In another aspect, the present disclosure provides a compound of formula (If), or a salt, solvate, isotopologue, prodrug, stereoisomer, or tautomer thereof, or any mixtures thereof, wherein L¹, L², R^{3a}, R^{3b}, R⁴, and R⁵ are defined elsewhere herein:

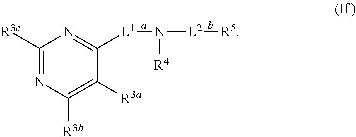

In another aspect, the present disclosure provides a compound of formula (Ig), or a salt, solvate, isotopologue, prodrug, stereoisomer, or tautomer thereof, or any mixtures thereof, wherein L¹, L², R^{3a}, R^{3b}, R^{3c}, R^{3d}, R^{3e}, R⁴, and R⁵ are defined elsewhere herein are defined elsewhere herein:

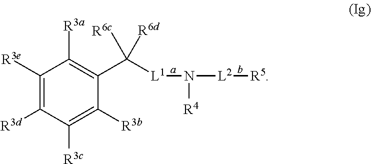

In another aspect, the present disclosure provides a compound of formula (IT), or a salt, solvate, isotopologue, prodrug, stereoisomer, or tautomer thereof, or any mixtures thereof, wherein A², L³, Z¹, Z², Z³, Z⁴, X², and R¹⁰ are defined elsewhere herein:

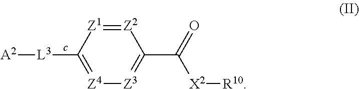

In another aspect, the present disclosure provides a compound of formula (III), or a salt, solvate, isotopologue, prodrug, stereoisomer, or tautomer thereof, or any mixtures thereof, wherein A³, A⁴, R^{15a}, R^{15b}, R^{15c}, R¹⁶, R^{17a}, R^{17b}, R^{18a}, and R^{18b} are defined elsewhere herein.

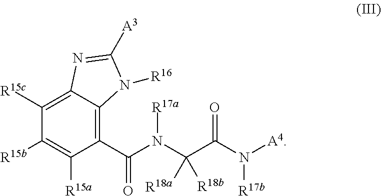

In another aspect, the present disclosure provides a pharmaceutical composition comprising at least one compound of formula (I), (Ia), (Ib), (Ic), (Id), (Ie), (If), (Ig), (II), or (III) and a pharmaceutically acceptable carrier.

In another aspect, the present disclosure provides a method of treating, preventing, and/or ameliorating endometriosis in a subject in need thereof. In certain embodiments, the method comprises administering to the subject at least one Neurotrophic Receptor Tyrosine Kinase (NTRK) inhibitor or a pharmaceutical composition comprising the NTRK inhibitor and a pharmaceutically acceptable carrier.

In another aspect, the present disclosure provides a method of treating, preventing, and/or ameliorating cancer in a subject in need thereof. In certain embodiments, the method comprises administering to the subject at least one Neurotrophic Receptor Tyrosine Kinase (NTRK) inhibitor or a pharmaceutical composition comprising the NTRK inhibitor and a pharmaceutically acceptable carrier.

In another aspect, the present disclosure provides a method of treating, preventing, and/or ameliorating pain in a subject in need thereof. In certain embodiments, the method comprises administering to the subject at least one Neurotrophic Receptor Tyrosine Kinase (NTRK) inhibitor or a pharmaceutical composition comprising the NTRK inhibitor and a pharmaceutically acceptable carrier.

In certain embodiments, the NTRK inhibitor is a compound of formula (I), (Ia), (Ib), (Ic), (Id), (Ie), (If), (Ig), (II), or (III).

the NTRK2 inhibitor is selected from the group consisting of staurosporine, entrectinib, selitrectinib, reprotrectinib, and PF-06273340 In certain embodiments, the NTRK inhibitor is a NTRK2 inhibitor.

In certain embodiments, the NTRK2 inhibitor selectively inhibits NTRK2 over NTRK1 and/or NTRK3. In certain embodiments, the NTRK2 inhibitor selectively inhibits NTRK2 over NTRK1. In certain embodiments, the NTRK2 inhibitor selectively inhibits NTRK2 over NTRK3.

### BRIEF DESCRIPTION OF THE FIGURES

The drawings illustrate generally, by way of example, but not by way of limitation, various embodiments of the present application.

FIGS. 1A-1B show the identification of NTRK2 as a therapeutic target for endometriosis. NTRK2 overexpression was identified in NTRK2 isoforms NM_006180 (FIG. TA) and NM-001007097 (FIG. 1B) by gene expression profiling, which compared endometrial tissues obtained from normal women (control endometrium, CE, n=43), women with endometriosis (patient endometriosis, PE, n=104), and endometriosis lesions (ovarian endometriomas, OMA, n=28; deep-infiltrating endometriosis, DiE, n=91; peritoneal endometriosis lesions, PeL, n=79). NTRK2 overexpression was confirmed in three additional endometriosis datasets, GSE25628, GSE23339, GSE7305 (fold change >1.4, p<0.05).

FIGS. 2A-2K shows NTRK2 overexpression and activity in stromal cells of endometriotic lesions. FIG. 2A: Western blotting analysis showing that NTRK2 is more elevated in lysates from ectopic peritoneal lesions and ovarian endometriomas than in eutopic normal endometrium or in eutopic endometrium from endometriosis patients. FIGS. 2B-2G: Immunohistochemistry of NTRK2 and negative control (NC) shows staining in the stroma and epithelial cells in a peritoneal endometriosis lesion. Similar results were observed in endometriotic lesions from endometriomas and rectal endometriosis lesions. FIGS. 2H-2J: qRT-PCR shows that PGE2-mediated induction of the inflammatory genes, PTGS2/COX2, IL6, and IL8, are decreased following NTRK2 knockdown in primary stromal cells from endometriomas. FIG. 2K: Western blot analysis of endometriotic stromal cells treated with siNTRK2±PGE2 shows that PGE2 induced CREB phosphorylation is suppressed following NTRK2 siRNA treatment.

FIG. 3 demonstrates that NTRK2 variants are associated with endometriosis. Analysis of 36,697 endometriosis cases and 116,071 controls from the FinnGen database identified 20 NTRK2 variants associated with endometriosis (i.e., −log₁₀p values≥3; p<0.001). Note that 182 NTRK2 variants are found within 0.001<p<0.01). Variants within the NTRK2 gene boundaries are shaded darker than those upstream or downstream of the NTRK2 gene boundary.

FIG. 4 provides the sequence of steps for use of the DEC-Tec platform. (1) generate compounds attached to DNA using key chemical reactions; (2) prepare library pools of >6 billion DNA-encoded drug-like compounds, each with a “DNA barcode”; (3) affinity selection of the library against his-tagged proteins; (4) capture his-tagged proteins with small molecules bound to the proteins with nickel beads; (5) separate proteins from unbound compounds; (6) sequencing and informatics analysis of DNA barcodes to decode the molecular structures of putative selective, drug-like “Hits”; and (7) re-synthesize “Hits” with/without DNA tag to test binding/inhibition.

FIGS. 5A-5B show non-limiting use of a DEC-Tec platform for identification of NTRK2 binders. Parallel DEC-Tec selections were performed with NTRK2, without NTRK2 (Control), or in the presence of promiscuous kinase inhibitor, staurosporine (not shown). Enrichment of each library member (measured by a normalized z-score) from the selections were compared. A series of similar compounds was significantly enriched in the NTRK2-only selection, but not control or NTRK2+staurosporine selections, consistent with competitive ligand binding. Hit CDD-2262 and analog CDD-2330 are potent and metabolically stable molecules.

FIGS. 6A-6B show the live-cell engagement (FIG. 6A) concept and an NTRK2 example (FIG. 6B). FIG. 6A: Depiction of Promega's NanoBRET target engagement system as per their website. The system involves cell-based competitive displacement of a fluorescent NanoBRET tracer bound reversibly to a NanoLuc fusion protein. Test compounds displace the tracer dependent on their affinity for the target and reduce loss of the NanoBRET signal. FIG. 6B: Example of the use of Promega's NTRK2 NanoBRET cell-based system. Each NTRK2 compound (Cpd) was assayed in kinase assays and in cells. Multiple NTRK2 compounds enter the cell and are potent nanomolar inhibitors in these assays (% normalized to no Cpd). Staurosporine, a promiscuous kinase inhibitor, is a positive control in these assays.

FIGS. 7A-7B show a TREEspot™ representation of KINOMEscan binding data of CDD-2330 across the human kinome phylogenetic tree (FIG. 7A) and a Z'-LYTE™ kinase screening assay (FIG. 7B) which demonstrates the low nanomolar affinity of CDD-2330 for the NTRK subfamily. FIG. 7A: The larger the filled circles, the higher the affinity of ligand binding; CDD-2330 is highly specific for NTRK1, NTRK2, and NTRK3 even when tested at a concentration of 1 μM and has minimal off-target binding. FIG. 7B: Using the ThermoFisher Z'-LYTE™ screening assay, kinase specificity for the NTRK subfamily was observed.

FIGS. 8A-8B provide graphs demonstrating the metabolic stability of CDD-3031 in mouse liver microsomes (MLMs) (FIG. 8A) and human liver microsomes (FIG. 8B).

FIGS. 9A-9B show mouse brain (FIG. 9A) and testis (FIG. 9B) distribution of CDD-2330. Mice (n=3 per timepoint) were treated intraperitoneally with CDD-2330 and brain, testis, and blood samples were collected. Samples were processed and analyzed by LC-MS.

FIGS. 10A-10C show the distribution of hydroxyzine (FIG. 10A), chlorpromazine (FIG. 10B), and CDD-3031 (FIG. 10C) in the brain and plasma of mice 24 hours post-administration, demonstrating low brain penetration.

FIGS. 11A-11B shows pharmacokinetics of CDD-2330 (FIG. 11A) and CDD-3031 (FIG. 11B) in mice. CDD-2330 was dissolved in 10% DMSO+0.5% methyl cellulose at 5 mg/mL. CDD-2330 was orally delivered at 50 mg/kg to mice (n=4). Blood samples were collected at 0, 0.25, 0.5, 1, 2, 4, 6, 8, and 24 h. Samples were analyzed by LC-MS, and the concentration of CDD-2330 was quantified with a standard curve (range: 0-10 μM). CDD-3031 was administered by oral gavage at a dose of 50 mg/kg. Blood samples were collected at 0, 0.83, 0.167, 0.25, 0.5, 1, 2, 4, 6, 8, and 24 h.

FIGS. 12A-12C shows that NTRK2 fuels pro-inflammatory signaling and pain in endometriosis. FIG. 12A: estrogen, secreted by the ovary and endometriotic lesions, signals to macrophages and nerve fibers, including production of BDNF/NT-3 and pro-inflammatory cytokines, respectively. FIG. 12B: at the cellular level, our studies show that the pro-inflammatory prostaglandin E2 (PGE2) signals via the EP2/EP4 receptors on endometriotic stromal cells, inducing the phosphorylation and activation of CREB (pCREB). Activated CREB acts on the PTGS2 promoter, creating a positive feedback mechanism that results in elevated inflammation. PGE2 signaling also stimulates PKA/PKC signaling to induce the production of BDNF and NTF3 expression. FIG. 12C: endometriotic stromal cells were pre-treated with 1 μM entrectinib or various doses of CDD-2262 followed by stimulation with 1 μM PGE2 for 6 h. Gene expression of PTGS2 was quantified using quantitative real time PCR (RT-PCR).

FIG. 13 provides the chemical structures of metabolites of CDD-2330 in mouse plasma.

FIG. 14 provides a schematic depicting the experimental procedure for studies utilizing the endometriosis mouse model described herein.

FIGS. 15A-15E provide photographs (FIGS. 15A-15C) and graphs (FIG. 15D-15E) demonstrating that CDD-2737 decreases the number of lesions and the size of lesions in an endometriosis mouse model.

FIGS. 16A-16B provide graphs demonstrating that CDD-2737 provides no gross or serum effects on the liver and kidney.

FIGS. 17A-17F depict the robust efficacy of the inhibitory compounds of the present disclosure as NTRK2 inhibitors for inhibition of endometriotic lesion growth in mice utilizing CDD-3132 as an example. FIG. 17A provides a schematic overview of the experiment performed. FIGS. 17B-17C provide photographs showing excised lesions with administration of a control (FIG. 17B) or CDD-3132 (10 mg/kg/day) (FIG. 17C). FIGS. 17D-17F provide graphs which show the number of lesions (FIG. 17D), volume of lesions (FIG. 17E), and mass of lesions (FIG. 17F) per mouse with administration of certain compounds (i.e., CDD-3031, CDD-3119, CDD-3132, and PF-06273340 at 10 mg/kg/day) or controls (vehicle).

FIGS. 18A-18B provide microscopy images of excised lesions from mice administered vehicle or CDD-3132 by H&E staining (FIG. 18A) or confocal microscopy (FIG. 18B), demonstrating that NTRK2 inhibitors reduce proliferation and inflammation with endometriotic lesions.

FIG. 19 provides photographs of excised lesions from mice administered CDD-3031, CDD-3119, and PF-06273340 (10 mg/kg).

FIGS. 20A-20B provide graphs depicting total liver (FIG. 20A) and kidney (FIG. 20B) weight in mice administered certain compounds, demonstrating no significant impact of NTRK2 inhibitors on liver and kidney weights in mice treated for 14 days with certain compounds, including CDD-3031, CDD-3119, and CDD-3132.

### DETAILED DESCRIPTION OF THE DISCLOSURE

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms.

Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl.

Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R²NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “atm” as used herein refers to a pressure in atmospheres under standard conditions. Thus, 1 atm is a pressure of 101 kPa, 2 atm is a pressure of 202 kPa, and so on.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7.

Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-mnethyl-3,4-epoxycyclohexyl)propyl, 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl. 2-benzothiazolyl, 4-benzothiazolyl, 5-benzothiazolyl. 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein.

The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise.

Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “room temperature” as used herein refers to a temperature of about 15° C. to 28° C.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate).

Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

The term “staurosporine” refers to (5S,6R,7R,9S)-6-methoxy-5-methyl-7-(methylamino)-6,7,8,9,15,16-hexahydro-5H,14H-17-oxa-4b,9a,15-triaza-5,9-methanodibenzo[b,h]cyclonona[jkl]cyclopenta[e]-as-indacen-14-one.

The term “entrectinib” refers to N-(5-(3,5-difluorobenzyl)-1H-indazol-3-yl)-4-(4-methylpiperazin-1-yl)-2-((tetrahydro-2H-pyran-4-yl)amino)benzamide.

The terms “LOXO-101” and “larotrectinib” are used interchangeably herein to refer to (S)-N-(5-((R)-2-(2,5-difluorophenyl)pyrrolidin-1-yl)pyrazolo[1,5-a]pyrimidin-3-yl)-3-hydroxypyrrolidine-1-carboxamide.

The terms “LOXO-195” and “selitrectinib” are used interchangeably herein to refer to (6R,15R)-9-fluoro-15-methyl-2,11,16,20,21,24-hexazapentacyclo[16.5.2.02,6.07,12.021,25]pentacosa-1(24),7(12),8,10,18(25),19,22-heptaen-17-one or (13E,14E,22R,6R)-35-fluoro-6-methyl-7-aza-1(5,3)-pyrazolo[1,5-a]pyrimidina-3(3,2)-pyridina-2(1,2)-pyrrolidinacyclooctaphan-8-one.

The terms “TPX-005” and “reprotrectinib” are used interchangeably herein to refer to (3R,11S)-6-fluoro-3,11-dimethyl-10-oxa-2,13,17,18,21-pentazatetracyclo[13.5.2.04,9.018,22]docosa-1(21),4(9),5,7,15(22).16,19-heptaen-14-one or (13E,14E,3R,6S)-45-fluoro-3,6-dimethyl-5-oxa-2,8-diaza-1(5,3)-pyrazolo[1,5-a]pyrimidina-4(1,2)-benzenacyclononaphan-9-one.

The term “PF06273340” as used herein refers to N-[5-[[2-Amino-7-(2-hydroxy-1,1-dimethylethyl)-7H-pyrrolo[2,3-d]pyrimidin-5-yl]carbonyl]-3-pyridinyl]-5-chloro-2-pyridineacetamide.

### DESCRIPTION

Neurotrophic receptor tyrosine kinases 1, 2, and 3 (NTRK1, NTRK2, and NTRK3) have been shown to be critical for various aspects of nervous system function, including nociceptive pain. Gene fusions involving those kinases (e.g., TPM3-NTRKT, MPRIP-NTRK1, TRIM24-NTRK2, and ETV6-NTRK3) are observed in adult and pediatric gliomas and other cancers (e.g., non-small cell lung carcinoma), where they amplify signaling cascades and act as oncogenic drivers.

In one aspect, the present disclosure relates to the identification of targets for the treatment of patients with endometriosis, as well as the inflammation and pain associated with endometriosis. Accordingly, described herein are bioinformatic analyses of overexpressed kinases in endometriosis tissues, which resulted in the identification of NTRK2 as a potential therapeutic target for endometriosis.

As demonstrated herein, NTRK2 is upregulated in endometriosis lesions and silencing of NTRK2 in primary endometriotic stromal cells inhibits pro-inflammatory gene expression.

Further, administration of ANA-12, a selective NTRK2 inhibitor that crosses the blood-brain barrier rapidly to cause anti-depressant and anxiolytic effects, causes endometriotic lesion regression in mice. These studies indicate that NTRK2 contributes to the survival and inflammation associated with endometriosis.

The present disclosure further describes identification, preparation, and validation of NTRK2 inhibitors for the treatment, prevention, and/or amelioration of endometriosis, and/or symptoms thereof, cancer, and pain in a subject.

### Compounds

In one aspect, the present disclosure provides a compound of formula (I), or a salt, solvate, isotopologue, prodrug, stereoisomer, or tautomer thereof, or any mixtures thereof:

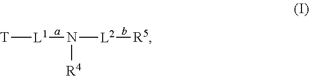

wherein:
T is selected from the group consisting of

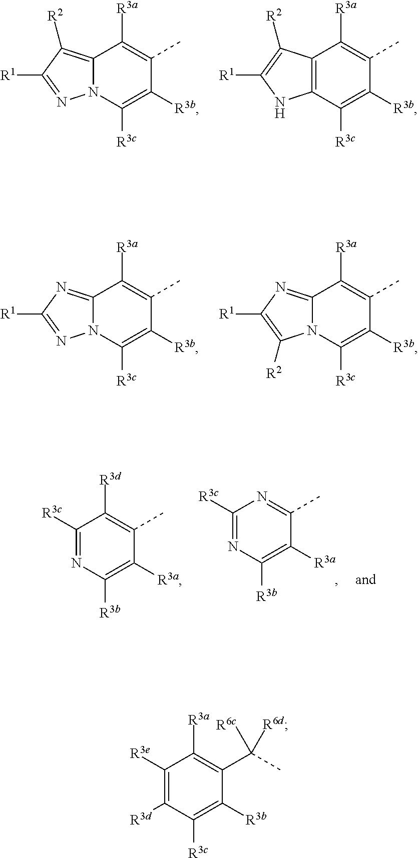

R¹ is selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R² is selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}) NR^{a}C(═O)R^{b}, C(═O)N(R^{a})(OR^{b}), C(═NR^{a})N(R^{b})(R^{c}), C(═NR^{a})N(R^{b})(OR^{c}), and NR^{a}S(═O)₂R^{b};
R^{3a}, R^{3b}, R^{3c}, R^{3d}, and R^{3e} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
R⁷ is selected from the group consisting of H and optionally substituted C₁-C₆ alkyl;
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
A¹ is selected from the group consisting of optionally substituted phenyl, optionally substituted naphthyl, and optionally substituted C₂-C₈ heterocyclyl;
R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
   or a salt, solvate, isotopologue, prodrug, stereoisomer, or tautomer thereof, or any mixtures thereof.

In certain embodiments, if T

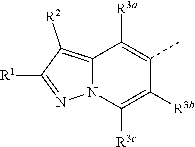

is and -L²-R⁵ is —H, then R⁴ is not H;

In another aspect, the present disclosure provides a compound of formula (Ia), or a salt, solvate, isotopologue, prodrug, stereoisomer, or tautomer thereof, or any mixtures thereof:

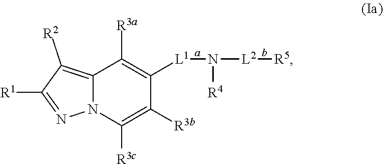

wherein:
R¹ is selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R² is selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}). NR^{a}C(═O)R^{b}, C(═O)N(R^{a})(OR^{b}), C(═NR^{a})N(R^{b})(R^{c}), C(═NR^{a})N(R^{b})(OR^{c}), and NR^{a}S(═O)₂R^{b};
R^{3a}, R^{3b}, and R^{3c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
L¹ is —C(R^{6a})(R^{6b})—;
X¹ is selected from the group consisting of a bond, O, and NR⁷;
R^{6a} and R^{6b} are each independently selected from the group consisting of H, optionally substituted C₁-C₆ alkyl, and C(═O)N(R^{a})(R^{b});
R⁷ is selected from the group consisting of H and optionally substituted C₁-C₆ alkyl;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
A¹ is selected from the group consisting of optionally substituted phenyl, optionally substituted naphthyl, and optionally substituted C₂-C₈ heterocyclyl; and
R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In certain embodiments, if -L²-R⁵ is —H, then R⁴ is not H.

In another aspect, the present disclosure provides a compound of formula (Ib), or a salt, solvate, isotopologue, prodrug, stereoisomer, or tautomer thereof, or any mixtures thereof:

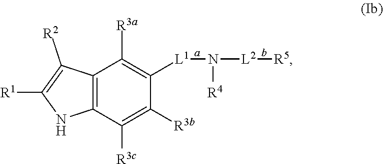

wherein:
R¹ is selected from the group consisting of H, optionally substituted C₁-C₆ alkyl, optionally substituted C₃-C₅ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R² is selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}). NR^{a}C(═O)R^{b}, C(═O)N(R^{a})(OR^{b}), C(═NR^{a})N(R^{b})(R^{c}), C(═NR^{a})N(R^{b})(OR^{c}), and NR^{a}S(═O)₂R^{b};
R^{3a}, R^{3A}, and R^{3c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a} and R^{6b} are each independently selected from the group consisting of H, optionally substituted C₁-C₆ alkyl, and C(═O)N(R^{a})(R^{b});
R⁷ is selected from the group consisting of H and optionally substituted C₁-C₆ alkyl;
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
A¹ is selected from the group consisting of optionally substituted phenyl, optionally substituted naphthyl, and optionally substituted C₂-C₈ heterocyclyl;
R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In another aspect, the present disclosure provides a compound of formula (Ic), or a salt, solvate, isotopologue, prodrug, stereoisomer, or tautomer thereof, or any mixtures thereof:

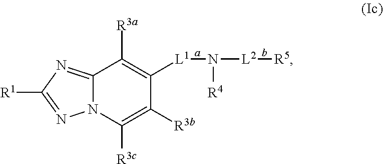

wherein:
R¹ is selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R^{3a}, R^{3b}, and R^{3c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
R⁷ is selected from the group consisting of H and optionally substituted C₁-C₆ alkyl;
A¹ is selected from the group consisting of optionally substituted phenyl, optionally substituted naphthyl, and optionally substituted C₂-C₈ heterocyclyl; R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In another aspect, the present disclosure provides a compound of formula (Id), or a salt, solvate, isotopologue, prodrug, stereoisomer, or tautomer thereof, or any mixtures thereof:

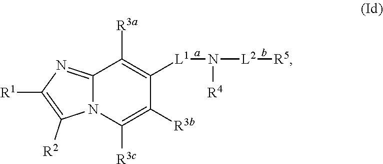

wherein:
R¹ is selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R² is selected from the group consisting of H, optionally substituted C₁-C₆ alkyl, optionally substituted C₃-C₅ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}). NR^{a}C(═O)R^{b}, C(═O)N(R^{a})(OR^{b}), C(═NR^{a})N(R^{b})(R^{c}), C(═NR^{a})N(R^{b})(OR^{c}), and NR^{a}S(═O)₂R^{b};
R^{3a}, R^{3b}, and R^{3c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
R⁷ is selected from the group consisting of H and optionally substituted C₁-C₆ alkyl;
A¹ is selected from the group consisting of optionally substituted phenyl, optionally substituted naphthyl, and optionally substituted C₂-C₈ heterocyclyl; R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In another aspect, the present disclosure provides a compound of formula (Ie), or a salt, solvate, isotopologue, prodrug, stereoisomer, or tautomer thereof, or any mixtures thereof:

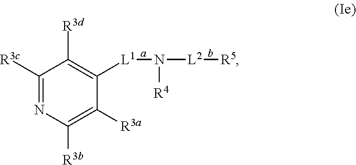

wherein:
R^{3a}, R^{3b}, R^{3c}, and R^{3d} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
R⁷ is selected from the group consisting of H and optionally substituted C₁-C₆ alkyl;
A¹ is selected from the group consisting of optionally substituted phenyl, optionally substituted naphthyl, and optionally substituted C₂-C₈ heterocyclyl;
   R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In another aspect, the present disclosure provides a compound of formula (If), or a salt, solvate, isotopologue, prodrug, stereoisomer, or tautomer thereof, or any mixtures thereof:

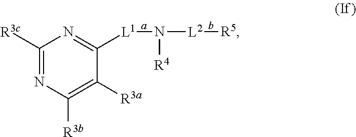

wherein:
R^{3a}, R^{3b}, and R^{3c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{a}, R^{6b}, R^{6c}, and R^{6d} are each independently selected from the group consisting of H, optionally substituted C₁-C₆ alkyl, and C(═O)N(R^{a})(R^{b});
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
R⁷ is selected from the group consisting of H and optionally substituted C₁-C₆ alkyl;
A¹ is selected from the group consisting of optionally substituted phenyl, optionally substituted naphthyl, and optionally substituted C₂-C₈ heterocyclyl;
   R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In another aspect, the present disclosure provides a compound of formula (Ig), or a salt, solvate, isotopologue, prodrug, stereoisomer, or tautomer thereof, or any mixtures thereof:

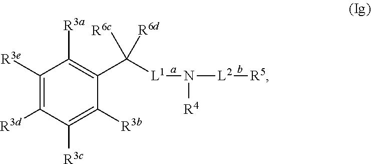

wherein:
R^{3a}, R^{3b}, R^{3c}, R^{3d}, and R^{3e} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
R⁷ is selected from the group consisting of H and optionally substituted C₁-C₆ alkyl;
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
A¹ is selected from the group consisting of optionally substituted phenyl, optionally substituted naphthyl, and optionally substituted C₂-C₈ heterocyclyl;
R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In certain embodiments, R¹ is H.

In certain embodiments, R² is H. In certain embodiments, R² is F. In certain embodiments, R² is Br. In certain embodiments, R² is CN. In certain embodiments, R² is C(═O)OH. In certain embodiments, R² is C(═O)NH₂. In certain embodiments, R² is C(═O)NMe. In certain embodiments, R² is C(═NH)NH(OH). In certain embodiments, R² is C(═O)NHOH.

In certain embodiments, at least one selected from R^{3a}, R^{3b}, and R^{3c} is H. In certain embodiments, at least two selected from R^{3a}, R^{3b} and R^{3c} are H. In certain embodiments, each of R^{3a}, R^{3b}, and R^{3c} are H. In certain embodiments, at least one selected from R^{3a}, R^{3b}R^{3c}, R^{3d}, and R^{3e} is H. In certain embodiments, at least two selected from R^{3a}, R^{3b}, R^{3c}, R^{3d}, and R^{3e} are H. In certain embodiments, at least three selected from R^{3a}, R^{3b}, R^{3c}, R^{3d}, and R^{3e} are H. In certain embodiments, at least four selected from R^{3a}, R^{3b}, R^{3c}, R^{3d}, and R^{3e} are H. In certain embodiments, each of R^{3a}, R^{3b}, R^{3c}, R^{3d}, and R^{3e} are H.

In certain embodiments, T is

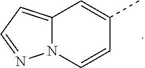

In certain embodiments, T is

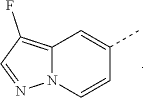

In certain embodiments, T is

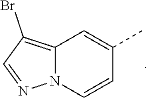

In certain embodiments, T is

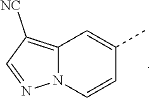

In certain embodiments, T is

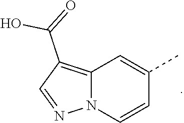

In certain embodiments, T is

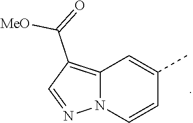

In certain embodiments, T is

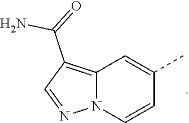

In certain embodiments, T is

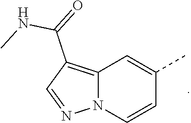

In certain embodiments, T is

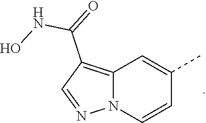

In certain embodiments, T is

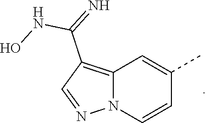

In certain embodiments, T is

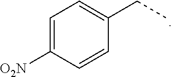

In certain embodiments, T is

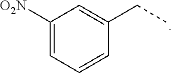

In certain embodiments, T is

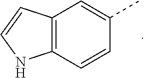

In certain embodiments, T is

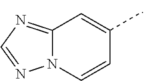

In certain embodiments, T is

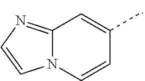

In certain embodiments, T is

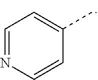

In certain embodiments, T is

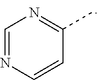

In certain embodiments, R⁴ is H. In certain embodiments, R⁴ is methyl. In certain embodiments, R⁴ is isopropyl. In certain embodiments, R⁴ is 1-methylpropyl. In certain embodiments, R⁴ is 2-methylpropyl. In certain embodiments, R⁴ is 3-methylbutyl. In certain embodiments, R⁴ is cyclopropyl. In certain embodiments, R⁴ is cyclobutyl. In certain embodiments, R⁴ is cyclopentyl. In certain embodiments, R⁴ is cyclohexyl. In certain embodiments, R⁴ is cycloheptyl. In certain embodiments, R⁴ is (CH₂)₃N(CH₃)₂. In certain embodiments, R⁴ is C(═O)CH₃. In certain embodiments, R⁴ is phenyl. In certain embodiments, R⁴ is benzyl substituted with at least one C₁-C₆ alkyl.

In certain embodiments, R⁴ is

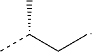

In certain embodiments, R⁴ is

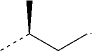

In certain embodiments, R⁴ is —CH₂CH₂CH₂O(phenyl substituted with at least one halogen). In certain embodiments, R⁴ is

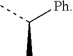

In certain embodiments, R⁴ is

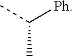

In certain embodiments, R⁴ is

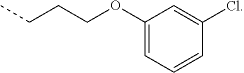

In certain embodiments, A¹ is H. In certain embodiments, A¹ is phenyl substituted with at least one C₁-C₆ alkyl. In certain embodiments, A¹ is phenyl substituted with at least one C₁-C₆ alkoxy. In certain embodiments, A¹ is phenyl substituted with at least one C₁-C₃ haloalkyl. In certain embodiments, A¹ is phenyl substituted with at least one C₁-C₃ haloalkoxy. In certain embodiments, A¹ is phenyl substituted with at least one phenoxy. In certain embodiments, A¹ is phenyl substituted with at least one halogen. In certain embodiments, A¹ is phenyl substituted with at least one NO₂. In certain embodiments, A¹ is phenyl substituted with at least one C₃-C₈ cycloalkyl, which is optionally substituted with at least one C₁-C₃ haloalkyl. In certain embodiments, A¹ is C₂-C₈ heteroaryl. In certain embodiments, A¹ is C₂-C₈ heteroaryl substituted with at least one C₁-C₆ alkyl. In certain embodiments, A¹ is C₂-C₈ heteroaryl fused with a C₂-C₈ heteroaryl. In certain embodiments, A¹ is

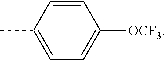

In certain embodiments, A¹ is

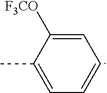

In certain embodiments, A¹ is

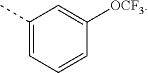

In certain embodiments, A¹ is

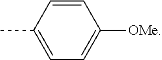

In certain embodiments, A¹ is

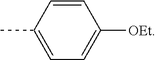

In certain embodiments, A¹ is

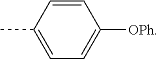

In certain embodiments, A¹ is

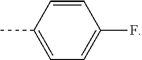

In certain embodiments, A¹ is

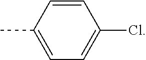

In certain embodiments, A¹ is

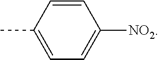

In certain embodiments, A¹ is

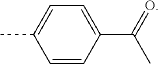

In certain embodiments, A¹ is

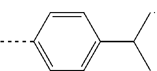

In certain embodiments, A¹ is

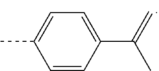

In certain embodiments, A¹ is

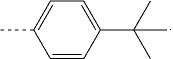

In certain embodiments, A¹ is

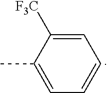

In certain embodiments, A¹ is

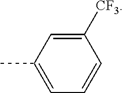

In certain embodiments, A¹ is

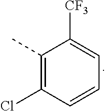

In certain embodiments, A¹ is

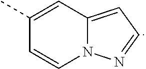

In certain embodiments, A¹ is

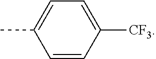

In certain embodiments, A¹ is

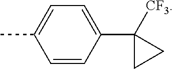

In certain embodiments, A¹ is

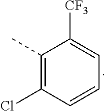

In certain embodiments, A¹ is

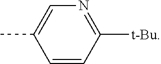

In certain embodiments, A¹ is

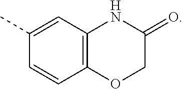

In certain embodiments, R^{6a} is H. In certain embodiments, R^{6b} is H.

In certain embodiments, L¹ is —CH₂—.

In certain embodiments, L² is a bond. In certain embodiments, L² is —C(═O)—. In certain embodiments, L² is —S(═O)₂—. In certain embodiments, L² is —C(═S)—. In certain embodiments, L² is —CH₂—. In certain embodiments, L² is

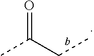

In certain embodiments, L² is

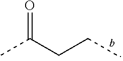

In certain embodiments, L² is

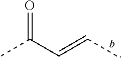

In certain embodiments, X¹ is NR⁷. In certain embodiments, X¹ is —NH— In certain embodiments, X¹ is NMe. In certain embodiments, X¹ is O. In certain embodiments, X¹ is a bond.

In certain embodiments, R⁷ is H. In certain embodiments, R⁷ is Me. In certain embodiments, R⁵ is H. In certain embodiments, R⁵ is

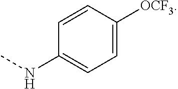

In certain embodiments, R⁵ is

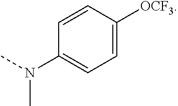

In certain embodiments, R⁵ is

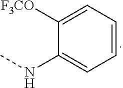

In certain embodiments, R⁵ is

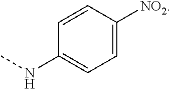

In certain embodiments, R⁵ is

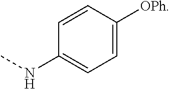

In certain embodiments, R⁵ is

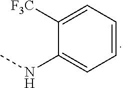

In certain embodiments, R⁵ is

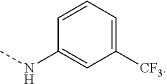

In certain embodiments, R⁵ is

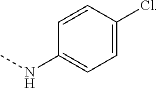

In certain embodiments, R⁵ is

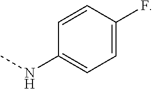

In certain embodiments, R⁵ is

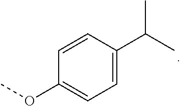

In certain embodiments, R⁵ is

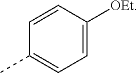

In certain embodiments, R⁵ is

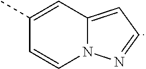

In certain embodiments, R⁵ is

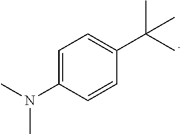

In certain embodiments, R⁵ is

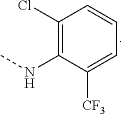

In certain embodiments, R⁵ is

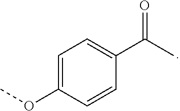

In certain embodiments, R⁵ is

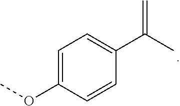

In certain embodiments, R⁵ is

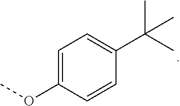

In certain embodiments, R⁵ is

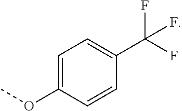

In certain embodiments, R⁵ is

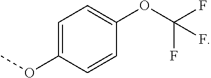

In certain embodiments, R⁵ is

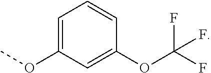

In certain embodiments, R⁵ is

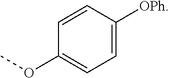

In certain embodiments, R⁵ is

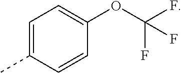

In certain embodiments, R⁵ is

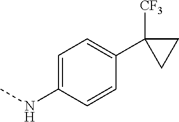

In certain embodiments, R⁵ is

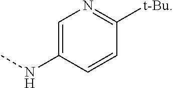

In certain embodiments, R⁵ is

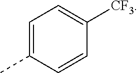

In certain embodiments, R⁵ is

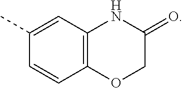

In certain embodiments, the compound of formula (I) is selected from the group consisting of
5-((1-isopropyl-3-(4-(trifluoromethoxy)phenyl)ureido)methyl)pyrazolo[1,5-a]pyridine-3-carboxylic acid;
1-cycloheptyl-1-(pyrazolo[1,5-a]pyridin-5-ylmethyl)-3-(4-(trifluoromethoxy)phenyl)urea;
1-cyclobutyl-1-(pyrazolo[1,5-a]pyridin-5-ylmethyl)-3-(2-(trifluoromethoxy)phenyl)urea;
1-isopropyl-1-(pyrazolo[1,5-a]pyridin-5-ylmethyl)-3-(4-(trifluoromethoxy)phenyl)urea;
1-cyclohexyl-1-(pyrazolo[1,5-a]pyridin-5-ylmethyl)-3-(4-(trifluoromethoxy)phenyl)urea;
1-cyclobutyl-3-(4-nitrophenyl)-1-(pyrazolo[1,5-a]pyridin-5-ylmethyl)urea;
1-phenyl-1-(pyrazolo[1,5-a]pyridin-5-ylmethyl)-3-(4-(trifluoromethoxy)phenyl)urea;
1-cyclopentyl-1-(pyrazolo[1,5-a]pyridin-5-ylmethyl)-3-(4-(trifluoromethoxy)phenyl)urea;
1-cyclopropyl-1-(pyrazolo[1,5-a]pyridin-5-ylmethyl)-3-(4-(trifluoromethoxy)phenyl)urea;
1-cyclobutyl-1-(pyrazolo[1,5-a]pyridin-5-ylmethyl)-3-(2-(trifluoromethyl)phenyl)urea;
1-cyclobutyl-1-(pyrazolo[1,5-a]pyridin-5-ylmethyl)-3-(3-(trifluoromethyl)phenyl)urea;
N-(3-(3-chlorophenoxy)propyl)-2-(4-isopropylphenoxy)-N-(pyrazolo[1,5-a]pyridin-5-ylmethyl)acetamide;
methyl 5-((cyclobutylamino)methyl)pyrazolo[1,5-a]pyridine-3-carboxylate;
3-(3-chlorophenoxy)-N-(pyrazolo[1,5-a]pyridin-5-ylmethyl)propan-1-amine;
N,N-bis(pyrazolo[1,5-a]pyridin-5-ylmethyl)cyclobutanamine:
1-(3-(3-chlorophenoxy)propyl)-3-(4-phenoxyphenyl)-1-(pyrazolo[1,5-a]pyridin-5-ylmethyl)urea;
1-(3-(3-chlorophenoxy)propyl)-1-(pyrazolo[1,5-a]pyridin-5-ylmethyl)-3-(4-(trifluoromethoxy)phenyl)urea;
3-(4-chlorophenyl)-1-cyclobutyl-1-(pyrazolo[1,5-a]pyridin-5-ylmethyl)urea;
1-cyclobutyl-3-(4-fluorophenyl)-1-(pyrazolo[1,5-a]pyridin-5-ylmethyl)urea;
5-((1-cyclobutyl-3-(4-(trifluoromethoxy)phenyl)ureido)methyl)-N-methylpyrazolo[1,5-a]pyridine-3-carboxamide;
5-((1-cyclobutyl-3-(4-(trifluoromethoxy)phenyl)ureido)methyl)pyrazolo[1,5-a]pyridine-3-carboxylic acid;
methyl 5-((1-cyclobutyl-3-(4-(trifluoromethoxy)phenyl)ureido)methyl)pyrazolo[1,5-a]pyridine-3-carboxylate;
1-cyclobutyl-3-(4-methoxyphenyl)-1-(pyrazolo[1,5-a]pyridin-5-ylmethyl)urea;
(R)-2-(4-ethoxyphenyl)-N-(1-phenylethyl)-N-(pyrazolo[1,5-a]pyridin-5-ylmethyl)acetamide;
(S)-2-(4-ethoxyphenyl)-N-(1-phenylethyl)-N-(pyrazolo[1,5-a]pyridin-5-ylmethyl)acetamide;
1-cyclobutyl-1-(pyrazolo[1,5-a]pyridin-5-ylmethyl)-3-(4-(trifluoromethoxy)phenyl)urea;
(S)-1-phenyl-N-(pyrazolo[1,5-a]pyridin-5-ylmethyl)ethan-1-amine;
1-((3-fluoropyrazolo[1,5-a]pyridin-5-yl)methyl)-1-isopropyl-3-(4-(trifluoromethoxy)phenyl)urea;
1-((3-bromopyrazolo[1,5-a]pyridin-5-yl)methyl)-1-isopropyl-3-(4-(trifluoromethoxy)phenyl)urea;
5-((1-cyclopropyl-3-(4-(trifluoro methoxy)phenyl) ureido)methyl)pyrazolo[1,5-a]pyridine-3-carboxylic acid;
5-((1-isopropyl-3-(4-(trifluoro methoxy)phenyl) ureido)methyl)pyrazolo[1,5-a]pyridine-3-carboxamide;
5-((1-cyclopropyl-3-(4-(trifluoro methoxy)phenyl) ureido)methyl)pyrazolo[1,5-a]pyridine-3-carboxamide;
5-((1-isopropyl-3-methyl-3-(4-(trifluoro methoxy) phenyl)ureido)methyl)pyrazolo[1,5-a]pyridine-3-carboxylic acid;
N-hydroxy-5-((1-isopropyl-3-methyl-3-(4-(trifluoro methoxy)phenyl)ureido)methyl)pyrazolo[1,5-a]pyridine-3-carboxamide;
1-((3-cyanopyrazolo[1,5-a]pyridin-5-yl)methyl)-1-isopropyl-3-(4-(trifluoromethoxy)phenyl)urea;
5-((1-isobutyl-3-(4-(trifluoromethoxy)phenyl)ureido)methyl)pyrazolo[1,5-a]pyridine-3-carboxylic acid;
5-((1-(sec-butyl)-3-(4-(trifluoromethoxy)phenyl) ureido)methyl)pyrazolo[1,5-a]pyridine-3-carboxylic acid;
N-hydroxy-5-((1-isopropyl-3-(4-(trifluoromethoxy) phenyl)ureido)methyl)pyrazolo[1,5-a]pyridine-3-carboximidamide;
5-((1-methyl-3-(4-(trifluoromethoxy)phenyl)thioureido)methyl)pyrazolo[1,5-a]pyridine-3-carboxamide;
5-((1-isopropyl-3-(4-(trifluoromethoxy)phenyl)thioureido)methyl)pyrazolo[1,5-a]pyridine-3-carboxamide;
5-((2-(4-isopropylphenoxy)-N-methylacetamido)methyl)pyrazolo[1,5-a]pyridine-3-carboxamide;
5-((N-methyl-3-(4-(trifluoromethoxy)phenyl)propanamido)methyl)pyrazolo[1,5-a]pyridine-3-carboxamide;
5-((1-methyl-3-(4-(trifluoromethoxy)phenyl)ureido)methyl)pyrazolo[1,5-a]pyridine-3-carboxamide;
5-((N-isopropyl-2-(4-isopropylphenoxy)acetamido)methyl)pyrazolo[1,5-a]pyridine-3-carboxamide;
5-((N-(3-(dimethylamino)propyl)-2-(4-isopropylphenoxy)acetamido)methyl)pyrazolo[1,5-a]pyridine-3-carboxamide;
5-((2-(4-isopropylphenoxy)acetamido)methyl)pyrazolo[1,5-a]pyridine-3-carboxamide;
5-((3-(4-(trifluoromethoxy)phenyl)ureido)methyl)pyrazolo[1,5-a]pyridine-3-carboxamide;
5-((3-(4-(trifluoromethoxy)phenyl)thioureido)methyl)pyrazolo[1,5-a]pyridine-3-carboxamide;
5-((N-methyl-2-(4-(trifluoromethoxy)phenoxy)acetamido)methyl)pyrazolo[1,5-a]pyridine-3-carboxamide;
5-((N-isopropyl-2-(4-(trifluoromethoxy)phenoxy)acetamido)methyl)pyrazolo[1,5-a]pyridine-3-carboxamide;
5-((1-(sec-butyl)-3-(4-(trifluoromethoxy)phenyl)ureido)methyl)pyrazolo[1,5-a]pyridine-3-carboxamide;
(R)-5-((1-(sec-butyl)-3-(4-(trifluoromethoxy)phenyl)ureido)methyl)pyrazolo[1,5-a]pyridine-3-carboxamide;
(S)-5-((1-(sec-butyl)-3-(4-(trifluoromethoxy)phenyl)ureido)methyl)pyrazolo[1,5-a]pyridine-3-carboxamide;
5-((N-methyl-2-(4-(trifluoromethyl)phenoxy)acetamido)methyl)pyrazolo[1,5-a]pyridine-3-carboxamide;
5-((N-methyl-2-(3-(trifluoromethoxy)phenoxy)acetamido)methyl)pyrazolo[1,5-a]pyridine-3-carboxamide;
5-((2-(4-(tert-butyl)phenoxy)-N-methylacetamido)methyl)pyrazolo[1,5-a]pyridine-3-carboxamide;
5-((2-(4-(tert-butyl)phenoxy)-N-isopropylacetamido)methyl)pyrazolo[1,5-a]pyridine-3-carboxamide;
5-((2-((4-(tert-butyl)phenyl)(methyl)amino)-N-methylacetamido)methyl)pyrazolo[1,5-a]pyridine-3-carboxamide;
5-((N-isopropyl-2-((4-(trifluoromethoxy)phenyl)amino)acetamido)methyl)pyrazolo[1,5-a]pyridine-3-carboxamide;
5-((N-isopropyl-2-(4-(prop-1-en-2-yl)phenoxy)acetamido)methyl)pyrazolo[1,5-a]pyridine-3-carboxamide;
5-((2-(4-acetylphenoxy)-N-isopropylacetamido)methyl)pyrazolo[1,5-a]pyridine-3-carboxamide;
5-((2-(4-acetylphenoxy)-N-methylacetamido)methyl)pyrazolo[1,5-a]pyridine-3-carboxamide;
5-((N-methyl-2-((4-(trifluoromethoxy)phenyl)amino)acetamido)methyl)pyrazolo[1,5-a]pyridine-3-carboxamide;
2-(3-(2-chloro-6-(trifluoromethyl)phenyl)ureido)-N,N-dimethyl-3-(3-nitrophenyl)propenamide;
(S)-2-(3-(2-chloro-6-(trifluoromethyl)phenyl)ureido)-N,N-dimethyl-3-(3-nitrophenyl)propenamide;
(R)-2-(3-(2-chloro-6-(trifluoromethyl)phenyl)ureido)-N,N-dimethyl-3-(3-nitrophenyl)propenamide;
N-methyl-3-(4-nitrophenyl)-2-(3-(4-phenoxyphenyl)ureido)propenamide;
(R)-N-methyl-3-(4-nitrophenyl)-2-(3-(4-phenoxyphenyl)ureido)propenamide;
(S)-N-methyl-3-(4-nitrophenyl)-2-(3-(4-phenoxyphenyl)ureido)propenamide;
N-((1H-indol-5-yl)methyl)-N-isopropyl-2-(4-(trifluoromethoxy)phenoxy)acetamide;
1-((1H-indol-5-yl)methyl)-1-isopropyl-3-(4-(trifluoromethoxy)phenyl)urea;
2-((6-(tert-butyl)pyridin-3-yl)amino)-N-isopropyl-N-(pyrazolo[1,5-a]pyridin-5-ylmethyl)acetamide;
N-isopropyl-N-(pyrazolo[1,5-a]pyridin-5-ylmethyl)-2-((4-(1-(trifluoromethyl)cyclopropyl)phenyl)amino)acetamide;
5-((methyl(N-(4-(trifluoromethoxy)phenyl)sulfamoyl)amino)methyl)pyrazolo[1,5-a]pyridine-3-carboxamide;
1-([1,2,4]triazolo[1,5-a]pyridin-7-ylmethyl)-1-cyclopropyl-3-(4-(trifluoromethoxy)phenyl)urea;
1-cyclopropyl-1-(pyridin-4-ylmethyl)-3-(4-(trifluoromethoxy)phenyl)urea;
1-cyclopropyl-1-(pyrimidin-4-ylmethyl)-3-(4-(trifluoromethoxy)phenyl)urea;
1-cyclopropyl-1-(imidazo[1,2-a]pyridin-7-ylmethyl)-3-(4-(trifluoromethoxy)phenyl)urea;
5-((N-(3-(3-chlorophenoxy)propyl)-2-(4-isopropylphenoxy)acetamido)methyl)pyrazolo[1,5-a]pyridine-3-carboxamide;
5-((N-(4-(trifluoromethoxy)phenyl)acetamido)methyl)pyrazolo[1,5-a]pyridine-3-carboxamide;
5-(((N-isopropyl-4-(trifluoromethyl)phenyl)sulfonamido)methyl)pyrazolo[1,5-a]pyridine-3-carboxamide;
5-(((N-isopropyl-3-oxo-3,4-dihydro-2H-benzo[b][1,4]oxazine)-6-sulfonamido)methyl)pyrazolo[1,5-a]pyridine-3-carboxamide;
5-(((N-methyl-4-(trifluoromethoxy)phenyl)sulfonamido)methyl)pyrazolo[1,5-a]pyridine-3-carboxamide;
5-(((N-isopropyl-4-(trifluoromethoxy)phenyl)sulfonamido)methyl)pyrazolo[1,5-a]pyridine-3-carboxamide;
5-((N-isopropyl-3-(4-methoxyphenyl)acrylamido)methyl)pyrazolo[1,5-a]pyridine-3-carboxamide;
(E)-5-((N-isopropyl-3-(4-methoxyphenyl)acrylamido)methyl)pyrazolo[1,5-a]pyridine-3-carboxamide;
(Z)-5-((N-isopropyl-3-(4-methoxyphenyl)acrylamido)methyl)pyrazolo[1,5-a]pyridine-3-carboxamide;
5-((N-isopropyl-3-(4-(trifluoromethyl)phenyl)acrylamido)methyl)pyrazolo[1,5-a]pyridine-3-carboxamide;
(E)-5-((N-isopropyl-3-(4-(trifluoromethyl)phenyl)acrylamido)methyl)pyrazolo[1,5-a]pyridine-3-carboxamide; and
(Z)-5-((N-isopropyl-3-(4-(trifluoromethyl)phenyl)acrylamido)methyl)pyrazolo[1,5-a]pyridine-3-carboxamide.

In another aspect, the present disclosure provides a compound of formula (II), or a salt, solvate, isotopologue, prodrug, stereoisomer, or tautomer thereof, or any mixtures thereof:

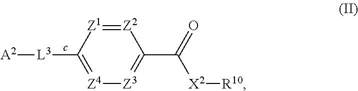

wherein:
A² is selected from the group consisting of

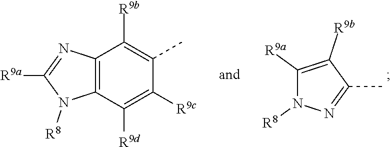

R⁸ is selected from the group consisting of H, optionally substituted C₁-C₆ alkyl, optionally substituted C₃-C₈ cycloalkyl, and optionally substituted phenyl;
R^{9a}, R^{9b}, R^{9c}, and R^{9d} are each independently selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, CN, and NO₂;
R¹⁰ is selected from the group consisting of optionally substituted C₁-C₆ alkyl,

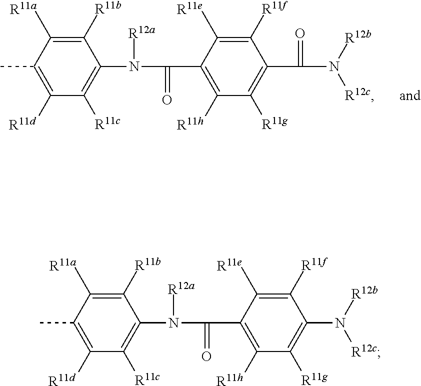

R^{11a}, R^{11b}, R^{11c}, R^{11d}, R^{11e}, R^{11f}, R^{11g}, and R^{11h} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{a}, and NR^{a}S(═O)₂R^{b};
R^{12a}, R^{12b}, and R^{12c} are each independently selected from the group consisting of H, optionally substituted C₁-C₆ alkyl, optionally substituted C₃-C₈ cycloalkyl, optionally substituted benzyl, and optionally substituted phenyl;
L³ is selected from the group consisting of

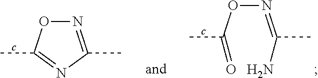

X² is selected from the group consisting of 0 and NR¹⁴;
R¹⁴ is selected from the group consisting of H and optionally substituted C₁-C₆ alkyl;
Z¹ is selected from the group consisting of CR¹³a and N. Z² is selected from the group consisting of CR^{13b} and N, Z³ is selected from the group consisting of CR^{13c} and N, and Z⁴ is selected from the group consisting of CR^{13d} and N,
   wherein at least one selected from the group consisting of Z¹, Z², Z³, and Z⁴ is N;
R^{13a}, R^{13b}, R^{13c}, and R^{13d} are each independently selected from the group consisting of H, optionally substituted C₁-C₆ alkyl, optionally substituted C₃-C₈ cycloalkyl, optionally substituted C₁-C₆ alkoxy, halogen, CN, and NO₂; and
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl.

In certain embodiments, R⁸ is isopropyl. In certain embodiments, R⁸ is phenyl.

In certain embodiments, R^{9a} is H. In certain embodiments, R^{9a} is tert-butyl. In certain embodiments, R^{9a} is trifluoromethyl. In certain embodiments, R^{9b} is H. In certain embodiments, R^{9b} is tert-butyl. In certain embodiments, R^{9b} is trifluoromethyl. In certain embodiments, R^{9c} is H. In certain embodiments, R^{9c} is tert-butyl. In certain embodiments, R^{9c} is trifluoromethyl. In certain embodiments, R^{9d} is H. In certain embodiments, R^{9d} is tert-butyl. In certain embodiments, R^{9d} is trifluoromethyl.

In certain embodiments, A² is

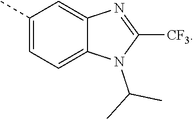

In certain embodiments, A² is

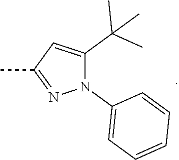

In certain embodiments, Z¹ is N and at least one of Z², Z³, and Z⁴ is CH. In certain embodiments, Z¹ is N and at least two of Z², Z³, and Z⁴ are CH. In certain embodiments, Z¹ is N and each of Z², Z³, and Z⁴ are CH. In certain embodiments, Z² is N and at least one of Z¹, Z³, and Z⁴ is CH. In certain embodiments, Z² is N and at least two of Z¹, Z³, and Z⁴ are CH. In certain embodiments, Z² is N and each of Z¹, Z³, and Z⁴ are CH.

In certain embodiments, X² is —O—. In certain embodiments, X² is —NH—.

In certain embodiments, R¹⁴ is H.

In certain embodiments, R^{11a} is H. In certain embodiments R^{11a} is methoxy. In certain embodiments, R^{11b} is H. In certain embodiments R^{11b} is methoxy. In certain embodiments, R^{11c} is H. In certain embodiments R^{11c} is methoxy. In certain embodiments, R^{11d} is H. In certain embodiments R^{11d} is methoxy. In certain embodiments, R^{11e} is H. In certain embodiments R^{11f} is methoxy. In certain embodiments, R^{11f} is H. In certain embodiments R^{11f} is methoxy. In certain embodiments, R^{11g} is H. In certain embodiments R^{11g} is methoxy. In certain embodiments, R^{11h} is H. In certain embodiments R^{11h} is methoxy.

In certain embodiments, R^{12a} is H. In certain embodiments, R^{12a} is methyl. In certain embodiments, R^{12a} is C(═O)O(C(CH₃)₃). In certain embodiments, R^{12b} is H. In certain embodiments, R^{12b} is methyl. In certain embodiments, R^{12b} is C(═O)O(C(CH₃)₃). In certain embodiments, R^{12c} is H. In certain embodiments, R^{12c} is methyl. In certain embodiments, R^{12c} is C(═O)O(C(CH₃)₃).

In certain embodiments R¹⁰ is tert-butyl. In certain embodiments, R¹⁰ is

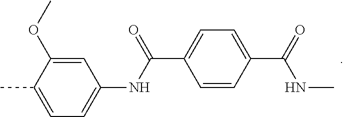

In certain embodiments, R¹⁰ is

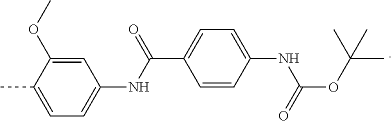

In certain embodiments, the compound of formula (II) is selected from the group consisting of
N¹-(4-(5-(5-(5-(tert-butyl)-1-phenyl-1H-pyrazol-3-yl)-1,2,4-oxadiazol-3-yl)picolinamido)-3-methoxyphenyl)-N⁴-methylterephthalamide;
N¹-(4-(5-(5-(1-isopropyl-2-(trifluoromethyl)-1H-benzo[d]imidazol-5-yl)-1,2,4-oxadiazol-3-yl)picolinamido)-3-methoxyphenyl)-N⁴-methylterephthalamide;
tert-butyl 5-(5-(5-(tert-butyl)-1-phenyl-TH-pyrazol-3-yl)-1,2,4-oxadiazol-3-yl)picolinate;
tert-butyl 5-(5-(1-isopropyl-2-(trifluoromethyl)-1H-benzo[d]imidazol-5-yl)-1,2,4-oxadiazol-3-yl)picolinate;
(Z)-N¹-(4-(5-(N′-((5-(tert-butyl)-1-phenyl-TH-pyrazole-3-carbonyl)oxy)carbamimidoyl)picolinamido)-3-methoxyphenyl)-N⁴-methylterephthalamide; and
tert-butyl (4-(5-(5-(1-isopropyl-2-(trifluoromethyl)-1H-benzo[d]imidazol-5-yl)-1,2,4-oxadiazol-3-yl)picolinamido)-3-methoxyphenyl)carbamate.

In another aspect, the present disclosure provides a compound of formula (III), or a salt, solvate, isotopologue, prodrug, stereoisomer, or tautomer thereof, or any mixtures thereof:

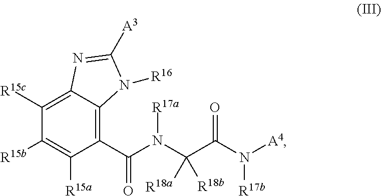

wherein:
R^{15a}, R^{15b}, and R^{15c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R¹⁶ is selected from the group consisting of H, optionally substituted C₁-C₆ alkyl, optionally substituted C₃-C₈ cycloalkyl, optionally substituted benzyl, and optionally substituted phenyl;
R^{17a} and R^{17b} are each independently selected from the group consisting of H, optionally substituted C₁-C₆ alkyl, optionally substituted C₃-C₈ cycloalkyl, optionally substituted benzyl, and optionally substituted phenyl;
R^{18a} and R^{18b} are each independently selected from the group consisting of H and optionally substituted C₁-C₆ alkyl;
A³ is optionally substituted phenyl; and
A⁴ is optionally substituted phenyl.

In certain embodiments, the compound of formula (III) is a compound of formula (IIIa):

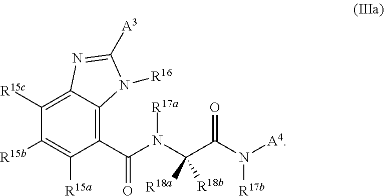

In certain embodiments, the compound of formula (III) is a compound of formula (IIIb):

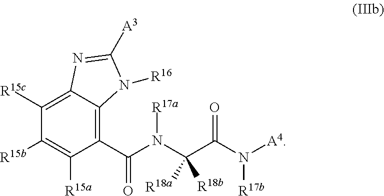

In certain embodiments, at least one of R^{15a}, R^{15b}, and R^{15c} is H. In certain embodiments, at least two of R^{15a}, R^{15b}, and R^{15c} are H. In certain embodiments, each of R^{15a}, R^{15b}, and R^{15c} are H.

In certain embodiments, R¹⁶ is benzyl. In certain embodiments, R¹⁶ is

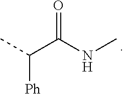

In certain embodiments, R^{17a} is H. In certain embodiments, R^{17b} is H.

In certain embodiments, R^{18a} is H. In certain embodiments, R^{18a} is Me. In certain embodiments, R^{18b} is H. In certain embodiments, R^{18b} is Me.

In certain embodiments, A³ is

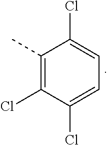

In certain embodiments, A³ is

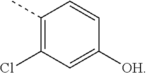

In certain embodiments, A⁴ is

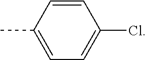

In certain embodiments, the compound of formula (III) is selected from the group consisting of
N-((S)-1-((4-chlorophenyl)amino)-1-oxopropan-2-yl)-1-(2-(methylamino)-2-oxo-1-phenylethyl)-2-(2,3,6-trichlorophenyl)-1H-benzo[d]imidazole-7-carboxamide;
(S)-1-benzyl-2-(2-chloro-4-hydroxyphenyl)-N-(1-((4-chlorophenyl)amino)-1-oxopropan-2-yl)-1H-benzo[d]imidazole-7-carboxamide; and
(S)-1-benzyl-N-(1-((4-chlorophenyl)amino)-1-oxopropan-2-yl)-2-(2,3,6-trichlorophenyl)-1H-benzo[d]imidazole-7-carboxamide.

In certain embodiments, each occurrence of optionally substituted alkyl, optionally substituted cycloalkyl, optionally substituted alkoxy, optionally substituted hydroxyalkyl, optionally substituted benzyl, optionally substituted phenyl, optionally substituted naphthyl, optionally substituted heterocyclyl, and optionally substituted alkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

TABLE 1
Exemplary compounds

| Cmpd No. | Compound |
|---|---|
| 1 CDD-2330 | 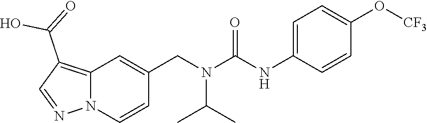 [SMILES: CC(C)N(Cc1ccn2ncc(C(=O)O)c2c1)C(=O)Nc1ccc(OC(F)(F)F)cc1] |
| | 5-((1-isopropyl-3-(4- |
| | (trifluoromethoxy)phenyl)ureido)methyl)pyrazolo[1,5-a]pyridine-3- |
| | carboxylic acid |
| 2 CDD-2301 | 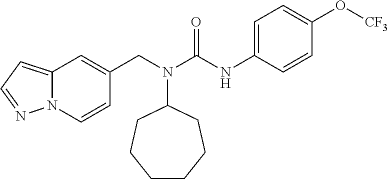 [SMILES: O=C(Nc1ccc(OC(F)(F)F)cc1)N(Cc1ccn2nccc2c1)C1CCCCCC1] |
| | 1-cycloheptyl-1-(pyrazolo[1,5-a]pyridin-5-ylmethyl)-3-(4- |
| | (trifluoromethoxy)phenyl)urea |
| 3 CDD-2300 | 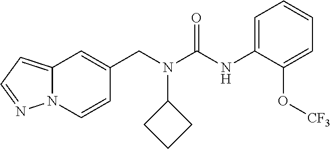 [SMILES: O=C(Nc1ccccc1OC(F)(F)F)N(Cc1ccn2nccc2c1)C1CCC1] |
| | 1-cyclobutyl-1-(pyrazolo[1,5-a]pyridin-5-ylmethyl)-3-(2- |
| | (trifluoromethoxy)phenyl)urea |
| 4 CDD-2299 | 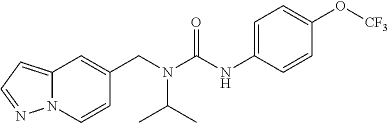 [SMILES: CC(C)N(Cc1ccn2nccc2c1)C(=O)Nc1ccc(OC(F)(F)F)cc1] |
| | 1-isopropyl-1-(pyrazolo[1,5-a]pyridin-5-ylmethyl)-3-(4- |
| | (trifluoromethoxy)phenyl)urea |
| 5 CDD-2298 | 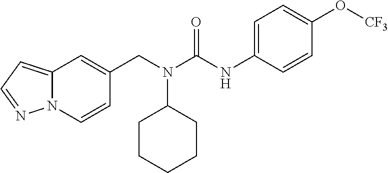 [SMILES: O=C(Nc1ccc(OC(F)(F)F)cc1)N(Cc1ccn2nccc2c1)C1CCCCC1] |
| | 1-cyclohexyl-1-(pyrazolo[1,5-a]pyridin-5-ylmethyl)-3-(4- |
| | (trifluoromethoxy)phenyl)urea |
| 6 CDD-2297 | 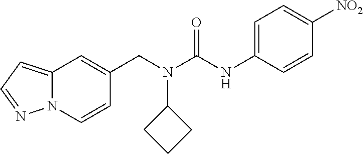 [SMILES: O=C(Nc1ccc([N+](=O)[O-])cc1)N(Cc1ccn2nccc2c1)C1CCC1] |
| | 1-cyclobutyl-3-(4-nitrophenyl)-1-(pyrazolo[1,5-a]pyridin-5-ylmethyl)urea |
| 7 CDD-2296 | 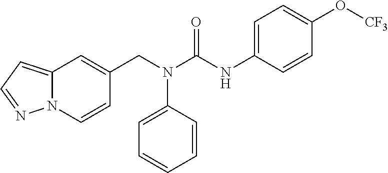 [SMILES: O=C(Nc1ccc(OC(F)(F)F)cc1)N(Cc1ccn2nccc2c1)c1ccccc1] |
| | 1-phenyl-1-(pyrazolo[1,5-a]pyridin-5-ylmethyl)-3-(4- |
| | (trifluoromethoxy)phenyl)urea |
| 8 CDD-2276 | 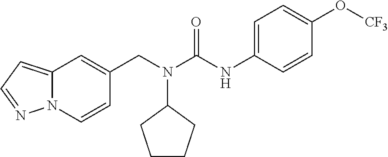 [SMILES: O=C(Nc1ccc(OC(F)(F)F)cc1)N(Cc1ccn2nccc2c1)C1CCCC1] |
| | 1-cyclopentyl-1-(pyrazolo[1,5-a]pyridin-5-ylmethyl)-3-(4- |
| | (trifluoromethoxy)phenyl)urea |
| 9 CDD-2275 | 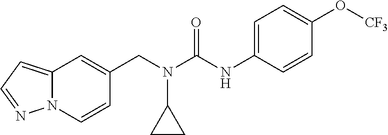 [SMILES: O=C(Nc1ccc(OC(F)(F)F)cc1)N(Cc1ccn2nccc2c1)C1CC1] |
| | 1-cyclopropyl-1-(pyrazolo[1,5-a]pyridin-5-ylmethyl)-3-(4- |
| | (trifluoromethoxy)phenyl)urea |
| 10 CDD-2274 | 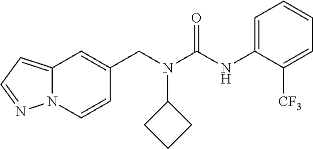 [SMILES: O=C(Nc1ccccc1C(F)(F)F)N(Cc1ccn2nccc2c1)C1CCC1] |
| | 1-cyclobutyl-1-(pyrazolo[1,5-a]pyridin-5-ylmethyl)-3-(2- |
| | (trifluoromethyl)phenyl)urea |
| 11 CDD-2273 | 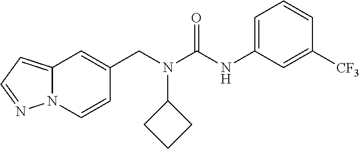 [SMILES: O=C(Nc1cccc(C(F)(F)F)c1)N(Cc1ccn2nccc2c1)C1CCC1] |
| | 1-cyclobutyl-1-(pyrazolo[1,5-a]pyridin-5-ylmethyl)-3-(3- |
| | (trifluoromethyl)phenyl)urea |
| 12 CDD-2271 | 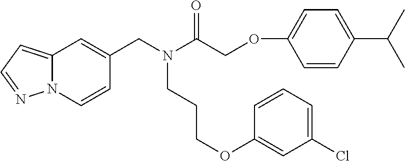 [SMILES: CC(C)c1ccc(OCC(=O)N(CCCOc2cccc(Cl)c2)Cc2ccn3nccc3c2)cc1] |
| | N-(3-(3-chlorophenoxy)propyl)-2-(4-isopropylphenoxy)-N-(pyrazolo[1,5- |
| | a]pyridin-5-ylmethyl)acetamide |
| 13 CDD-2270 | 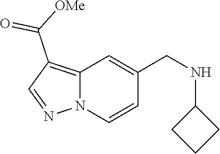 [SMILES: COC(=O)c1cnn2ccc(CNC3CCC3)cc12] |
| | methyl 5-((cyclobutylamino)methyl)pyrazolo[1,5-a]pyridine-3- |
| | carboxylate |
| 14 CDD-2269 | 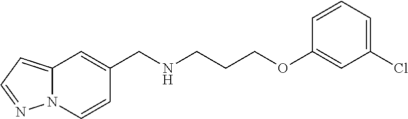 [SMILES: Clc1cccc(OCCCNCc2ccn3nccc3c2)c1] |
| | 3-(3-chlorophenoxy)-N-(pyrazolo[1,5-a]pyridin-5-ylmethyl)propan-1- |
| | amine |
| 15 CDD-2268 | 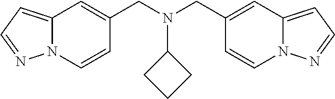 [SMILES: c1cc2cc(CN(Cc3ccn4nccc4c3)C3CCC3)ccn2n1] |
| | N,N-bis(pyrazolo[1,5-a]pyridin-5-ylmethyl)cyclobutanamine |
| 16 CDD-2267 | 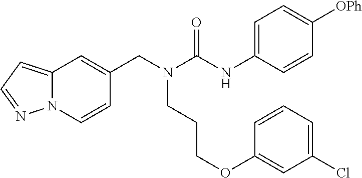 [SMILES: O=C(Nc1ccc(Oc2ccccc2)cc1)N(CCCOc1cccc(Cl)c1)Cc1ccn2nccc2c1] |
| | 1-(3-(3-chlorophenoxy)propyl)-3-(4-phenoxyphenyl)-1-(pyrazolo[1,5- |
| | a]pyridin-5-ylmethyl)urea |
| 17 CDD-2266 | 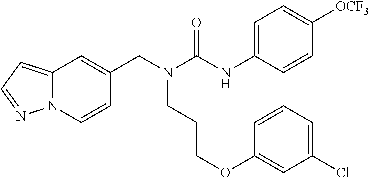 [SMILES: O=C(Nc1ccc(OC(F)(F)F)cc1)N(CCCOc1cccc(Cl)c1)Cc1ccn2nccc2c1] |
| | 1-(3-(3-chlorophenoxy)propyl)-1-(pyrazolo[1,5-a]pyridin-5-ylmethyl)-3- |
| | (4-(trifluoromethoxy)phenyl)urea |
| 18 CDD-2265 | 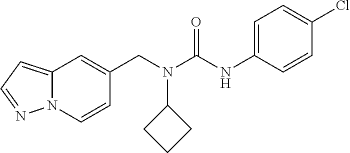 [SMILES: O=C(Nc1ccc(Cl)cc1)N(Cc1ccn2nccc2c1)C1CCC1] |
| | 3-(4-chlorophenyl)-1-cyclobutyl-1-(pyrazolo[1,5-a]pyridin-5- |
| | ylmethyl)urea |
| 19 CDD-2264 | 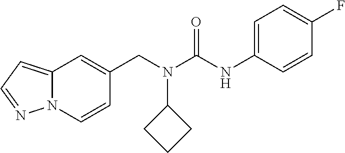 [SMILES: O=C(Nc1ccc(F)cc1)N(Cc1ccn2nccc2c1)C1CCC1] |
| | 1-cyclobutyl-3-(4-fluorophenyl)-1-(pyrazolo[1,5-a]pyridin-5- |
| | ylmethyl)urea |
| 20 CDD-2263 | 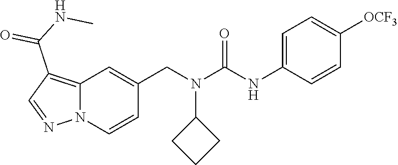 [SMILES: CNC(=O)c1cnn2ccc(CN(C(=O)Nc3ccc(OC(F)(F)F)cc3)C3CCC3)cc12] |
| | 5-((1-cyclobutyl-3-(4-(trifluoromethoxy)phenyl)ureido)methyl)-N- |
| | methylpyrazolo[1,5-a]pyridine-3-carboxamide |
| 21 CDD-2262 | 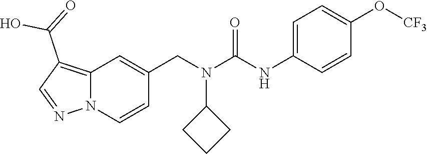 [SMILES: O=C(O)c1cnn2ccc(CN(C(=O)Nc3ccc(OC(F)(F)F)cc3)C3CCC3)cc12] |
| | 5-((1-cyclobutyl-3-(4- |
| | (trifluoromethoxy)phenyl)ureido)methyl)pyrazolo[1,5-a]pyridine-3- |
| | carboxylic acid |
| 22 CDD-2261 | 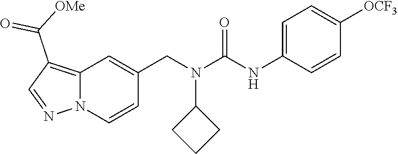 [SMILES: COC(=O)c1cnn2ccc(CN(C(=O)Nc3ccc(OC(F)(F)F)cc3)C3CCC3)cc12] |
| | methyl 5-((1-cyclobutyl-3-(4- |
| | (trifluoromethoxy)phenyl)ureido)methyl)pyrazolo[1,5-a]pyridine-3- |
| | carboxylate |
| 23 CDD-2260 | 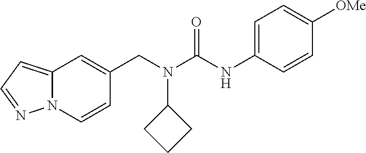 [SMILES: COc1ccc(NC(=O)N(Cc2ccn3nccc3c2)C2CCC2)cc1] |
| | 1-cyclobutyl-3-(4-methoxyphenyl)-1-(pyrazolo[1,5-a]pyridin-5- |
| | ylmethyl)urea |
| 24 CDD-2222 | 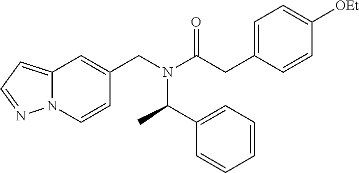 [SMILES: CCOc1ccc(CC(=O)N(Cc2ccn3nccc3c2)[C@H](C)c2ccccc2)cc1] |
| | (R)-2-(4-ethoxyphenyl)-N-(1-phenylethyl)-N-(pyrazolo[1,5-a]pyridin-5- |
| | ylmethyl)acetamide |
| 25 CDD-2259 | 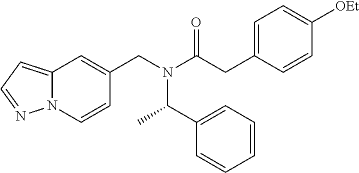 [SMILES: CCOc1ccc(CC(=O)N(Cc2ccn3nccc3c2)[C@@H](C)c2ccccc2)cc1] |
| | (S)-2-(4-ethoxyphenyl)-N-(1-phenylethyl)-N-(pyrazolo[1,5-a]pyridin-5- |
| | ylmethyl)acetamide |
| 26 CDD-2219 | 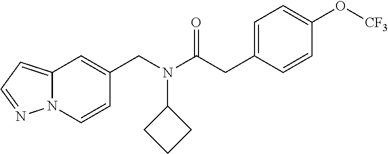 [SMILES: O=C(Cc1ccc(OC(F)(F)F)cc1)N(Cc1ccn2nccc2c1)C1CCC1] |
| | 1-cyclobutyl-1-(pyrazolo[1,5-a]pyridin-5-ylmethyl)-3-(4- |
| | (trifluoromethoxy)phenyl)urea |
| 27 CDD-2225 | 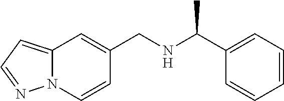 [SMILES: C[C@H](NCc1ccn2nccc2c1)c1ccccc1] |
| | (S)-1-phenyl-N-(pyrazolo[1,5-a]pyridin-5-ylmethyl)ethan-1-amine |
| 28 CDD-2734 | 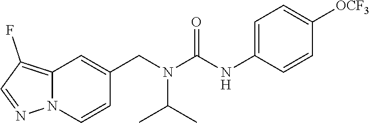 [SMILES: CC(C)N(Cc1ccn2ncc(F)c2c1)C(=O)Nc1ccc(OC(F)(F)F)cc1] |
| | 1-((3-fluoropyrazolo[1,5-a]pyridin-5-yl)methyl)-1-isopropyl-3-(4- |
| | (trifluoromethoxy)phenyl)urea |
| 29 CDD-2735 | 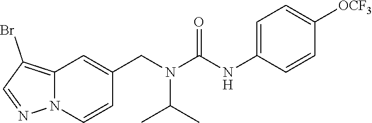 [SMILES: CC(C)N(Cc1ccn2ncc(Br)c2c1)C(=O)Nc1ccc(OC(F)(F)F)cc1] |
| | 1-((3-bromopyrazolo[1,5-a]pyridin-5-yl)methyl)-1-isopropyl-3-(4- |
| | (trifluoromethoxy)phenyl)urea |
| 30 CDD-2736 | 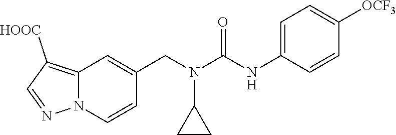 [SMILES: O=C(O)c1cnn2ccc(CN(C(=O)Nc3ccc(OC(F)(F)F)cc3)C3CC3)cc12] |
| | 5-((1-cyclopropyl-3-(4-(trifluoromethoxy)phenyl) |
| | ureido)methyl)pyrazolo[1,5-a]pyridine-3-carboxylic acid |
| 31 CDD-2737 | 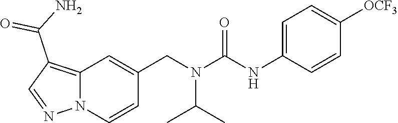 [SMILES: CC(C)N(Cc1ccn2ncc(C(N)=O)c2c1)C(=O)Nc1ccc(OC(F)(F)F)cc1] |
| | 5-((1-isopropyl-3-(4-(trifluoromethoxy)phenyl) |
| | ureido)methyl)pyrazolo[1,5-a]pyridine-3-carboxamide |
| 32 CDD-2738 | 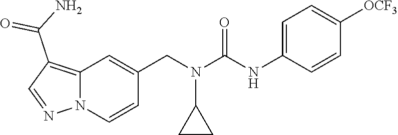 [SMILES: NC(=O)c1cnn2ccc(CN(C(=O)Nc3ccc(OC(F)(F)F)cc3)C3CC3)cc12] |
| | 5-((1-cyclopropyl-3-(4-(trifluoromethoxy)phenyl) |
| | ureido)methyl)pyrazolo[1,5-a]pyridine-3-carboxamide |
| 33 CDD-2739 | 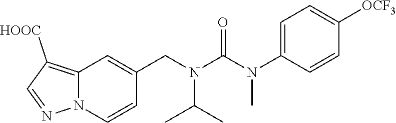 [SMILES: CC(C)N(Cc1ccn2ncc(C(=O)O)c2c1)C(=O)N(C)c1ccc(OC(F)(F)F)cc1] |
| | 5-((1-isopropyl-3-methyl-3-(4-(trifluoromethoxy) |
| | phenyl)ureido)methyl)pyrazolo[1,5-a]pyridine-3-carboxylic acid |
| 34 CDD-2740 | 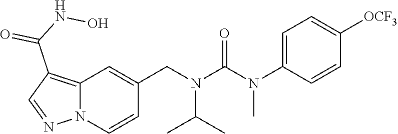 [SMILES: CC(C)N(Cc1ccn2ncc(C(=O)NO)c2c1)C(=O)N(C)c1ccc(OC(F)(F)F)cc1] |
| | N-hydroxy-5-((1-isopropyl-3-methyl-3-(4-trifluoro |
| | methoxy)phenyl)ureido)methyl)pyrazolo[1,5-a]pyridine-3-carboxamide |
| 35 CDD-2741 | 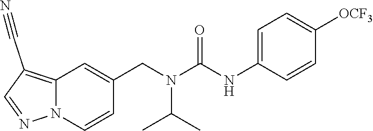 [SMILES: CC(C)N(Cc1ccn2ncc(C#N)c2c1)C(=O)Nc1ccc(OC(F)(F)F)cc1] |
| | 1-((3-cyanopyrazolo[1,5-a]pyridin-5-yl)methyl)-1-isopropyl-3-(4- |
| | (trifluoromethoxy)phenyl)urea |
| 36 CDD-2742 | 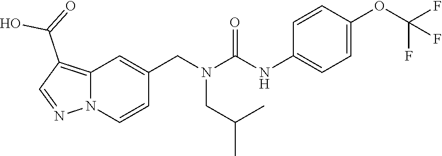 [SMILES: CC(C)CN(Cc1ccn2ncc(C(=O)O)c2c1)C(=O)Nc1ccc(OC(F)(F)F)cc1] |
| | 5-((1-isobutyl-3-(4-(trifluoromethoxy)phenyl)ureido)methyl)pyrazolo[1,5- |
| | a]pyridine-3-carboxylic acid |
| 37 CDD-2743 | 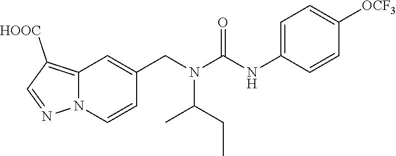 [SMILES: CCC(C)N(Cc1ccn2ncc(C(=O)O)c2c1)C(=O)Nc1ccc(OC(F)(F)F)cc1] |
| | 5-((1-(sec-butyl)-3-(4-(trifluoromethoxy)phenyl) |
| | ureido)methyl)pyrazolo[1,5-a]pyridine-3-carboxylic acid |
| 38 CDD-2744 | 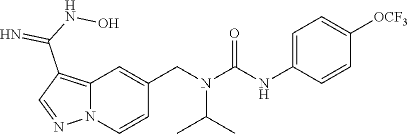 [SMILES: CC(C)N(Cc1ccn2ncc(C(=N)NO)c2c1)C(=O)Nc1ccc(OC(F)(F)F)cc1] |
| | N-hydroxy-5-((1-isopropyl-3-(4-(trifluoromethoxy) |
| | phenyl)ureido)methyl)pyrazolo[1,5-a]pyridine-3-carboximidamide |
| 39 CDD-2241 | 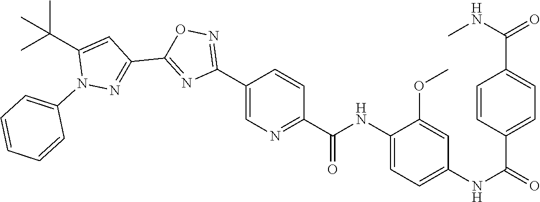 [SMILES: CNC(=O)c1ccc(C(=O)Nc2ccc(NC(=O)c3ccc(-c4noc(-c5cc(C(C)(C)C)n(-c6ccccc6)n5)n4)cn3)c(OC)c2)cc1] |
| | N¹-(4-(5-(5-(5-(tert-butyl)-1-phenyl-1H-pyrazol-3-yl)-1,2,4-oxadiazol-3- |
| | yl)picolinamido)-3-methoxyphenyl)-N⁴-methylterephthalamide |
| 40 CDD-2240 | 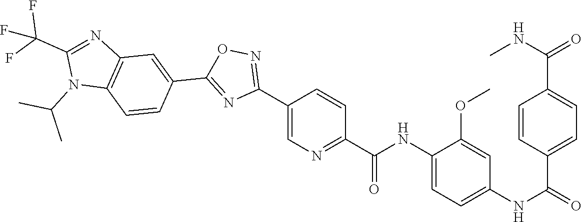 [SMILES: CNC(=O)c1ccc(C(=O)Nc2ccc(NC(=O)c3ccc(-c4noc(-c5ccc6c(c5)nc(C(F)(F)F)n6C(C)C)n4)cn3)c(OC)c2)cc1] |
| | N¹-(4-(5-(5-(1-isopropyl-2-(trifluoromethyl)-1H-benzo[d]imidazol-5-yl)- |
| | 1,2,4-oxadiazol-3-yl)picolinamido)-3-methoxyphenyl)-N⁴- |
| | methylterephthalamide |
| 41 CDD-2239 | 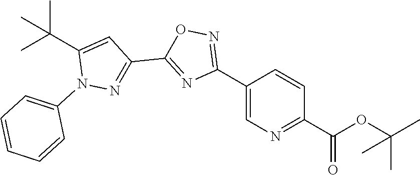 [SMILES: CC(C)(C)OC(=O)c1ccc(-c2noc(-c3cc(C(C)(C)C)n(-c4ccccc4)n3)n2)cn1] |
| | tert-butyl 5-(5-(5-(tert-butyl)-1-phenyl-1H-pyrazol-3-yl)-1,2,4-oxadiazol- |
| | 3-yl)picolinate |
| 42 CDD-2238 | 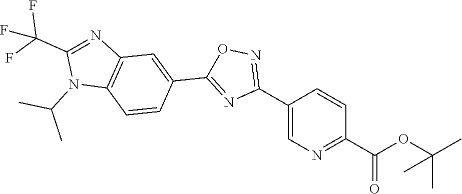 [SMILES: CC(C)n1c(C(F)(F)F)nc2cc(-c3nc(-c4ccc(C(=O)OC(C)(C)C)nc4)no3)ccc21] |
| | tert-butyl 5-(5-(1-isopropyl-2-(trifluoromethyl)-1H-benzo[d]imidazol-5- |
| | yl)-1,2,4-oxadiazol-3-yl)picolinate |
| 43 CDD-2228 | 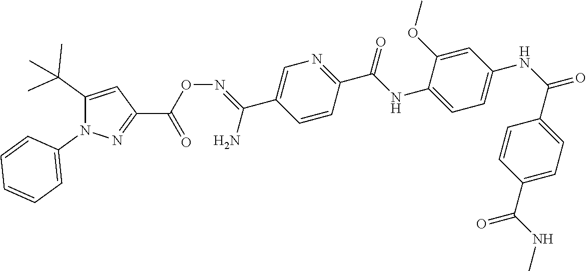 [SMILES: CNC(=O)c1ccc(C(=O)Nc2ccc(NC(=O)c3ccc(/C(N)=N/OC(=O)c4cc(C(C)(C)C)n(-c5ccccc5)n4)cn3)c(OC)c2)cc1] |
| | (Z)-N¹-(4-(5-(N¹-((5-(tert-butyl)-1-phenyl-1H-pyrazole-3- |
| | carbonyl)oxy)carbamimidoyl)picolinamido)-3-methoxyphenyl)-N⁴- |
| | methylterephthalamide |
| 44 CDD-2224 | 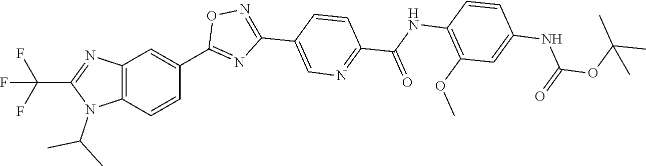 [SMILES: COc1cc(NC(=O)OC(C)(C)C)ccc1NC(=O)c1ccc(-c2noc(-c3ccc4c(c3)nc(C(F)(F)F)n4C(C)C)n2)cn1] |
| | tert-butyl (4-(5-(5-(1-isopropyl-2-(trifluoromethyl)-1H-benzo[d]imidazol- |
| | 5-yl)-1,2,4-oxadiazol-3-yl)picolinamido)-3-methoxyphenyl)carbamate |
| 45 CDD-2218 | 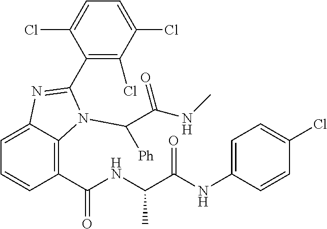 [SMILES: CNC(=O)C(c1ccccc1)n1c(-c2c(Cl)ccc(Cl)c2Cl)nc2cccc(C(=O)N[C@@H](C)C(=O)Nc3ccc(Cl)cc3)c21] |
| | N-((S)-1-((4-chlorophenyl)amino)-1-oxopropan-2-yl)-1-(2- |
| | (methylamino)-2-oxo-1-phenylethyl)-2-(2,3,6-trichlorophenyl)-1H- |
| | benzo[d]imidazole-7-carboxamide |
| 46 CDD-2217 | 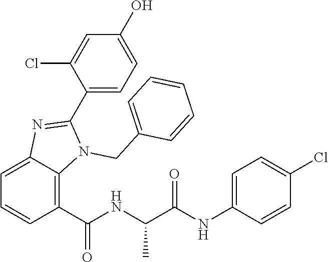 [SMILES: C[C@H](NC(=O)c1cccc2nc(-c3ccc(O)cc3Cl)n(Cc3ccccc3)c12)C(=O)Nc1ccc(Cl)cc1] |
| | (S)-1-benzyl-2-(2-chloro-4-hydroxyphenyl)-N-(1-((4- |
| | chlorophenyl)amino)-1-oxopropan-2-yl)-1H-benzo[d]imidazole-7- |
| | carboxamide |
| 47 CDD-2216 | 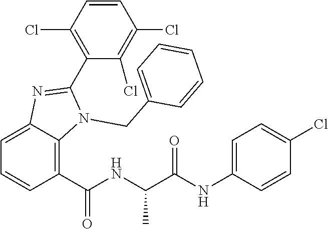 [SMILES: C[C@H](NC(=O)c1cccc2nc(-c3c(Cl)ccc(Cl)c3Cl)n(Cc3ccccc3)c12)C(=O)Nc1ccc(Cl)cc1] |
| | (S)-1-benzyl-N-(1-((4-chlorophenyl)amino)-1-oxopropan-2-yl)-2-(2,3,6- |
| | trichlorophenyl)-1H-benzo[d]imidazole-7-carboxamide |
| 48 CDD-3118 | 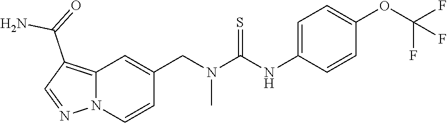 [SMILES: CN(Cc1ccn2ncc(C(N)=O)c2c1)C(=S)Nc1ccc(OC(F)(F)F)cc1] |
| | 5-((1-methyl-3-(4- |
| | (trifluoromethoxy)phenyl)thioureido)methyl)pyrazolo[1,5-a]pyridine-3- |
| | carboxamide |
| 49 CDD-3119 | 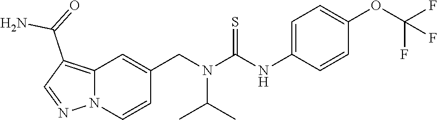 [SMILES: CC(C)N(Cc1ccn2ncc(C(N)=O)c2c1)C(=S)Nc1ccc(OC(F)(F)F)cc1] |
| | 5-((1-isopropyl-3-(4- |
| | (trifluoromethoxy)phenyl)thioureido)methyl)pyrazolo[1,5-a]pyridine-3- |
| | carboxamide |
| 50 CDD-3120 | 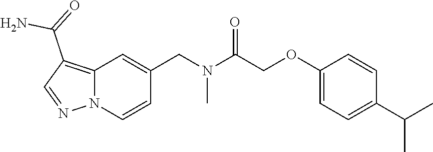 [SMILES: CC(C)c1ccc(OCC(=O)N(C)Cc2ccn3ncc(C(N)=O)c3c2)cc1] |
| | 5-((2-(4-isopropylphenoxy)-N-methylacetamido)methyl)pyrazolo[1,5- |
| | a]pyridine-3-carboxamide |
| 51 CDD-3121 | 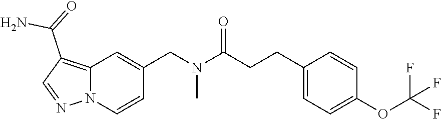 [SMILES: CN(Cc1ccn2ncc(C(N)=O)c2c1)C(=O)CCc1ccc(OC(F)(F)F)cc1] |
| | 5-((N-methyl-3-(4- |
| | (trifluoromethoxy)phenyl)propanamido)methyl)pyrazolo[1,5-a]pyridine- |
| | 3-carboxamide |
| 52 CDD-3031 | 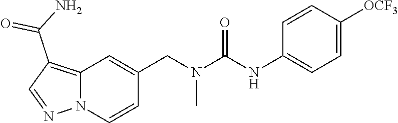 [SMILES: CN(Cc1ccn2ncc(C(N)=O)c2c1)C(=O)Nc1ccc(OC(F)(F)F)cc1] |
| | 5-((1-methyl-3-(4-(trifluoromethoxy)phenyl)ureido)methyl)pyrazolo[1,5- |
| | a]pyridine-3-carboxamide |
| 53 CDD-3032 | 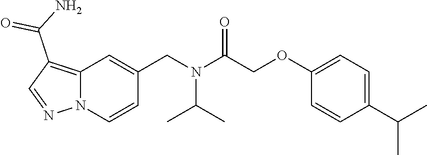 [SMILES: CC(C)c1ccc(OCC(=O)N(Cc2ccn3ncc(C(N)=O)c3c2)C(C)C)cc1] |
| | 5-((N-isopropyl-2-(4-isopropylphenoxy)acetamido)methyl)pyrazolo[1,5- |
| | a]pyridine-3-carboxamide |
| 54 CDD-3122 | 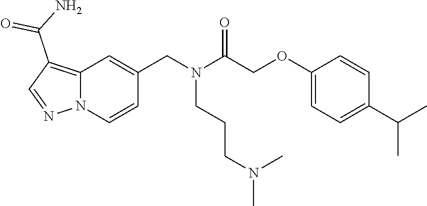 [SMILES: CC(C)c1ccc(OCC(=O)N(CCCN(C)C)Cc2ccn3ncc(C(N)=O)c3c2)cc1] |
| | 5-((N-(3-(dimethylamino)propyl)-2-(4- |
| | isopropylphenoxy)acetamido)methyl)pyrazolo[1,5-a]pyridine-3- |
| | carboxamide |
| 55 CDD-3129 | 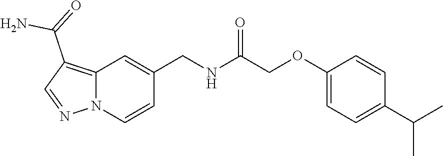 [SMILES: CC(C)c1ccc(OCC(=O)NCc2ccn3ncc(C(N)=O)c3c2)cc1] |
| | 5-((2-(4-isopropylphenoxy)acetamido)methyl)pyrazolo[1,5-a]pyridine-3- |
| | carboxamide |
| 56 CDD-3130 | 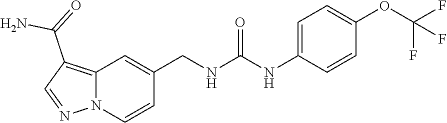 [SMILES: NC(=O)c1cnn2ccc(CNC(=O)Nc3ccc(OC(F)(F)F)cc3)cc12] |
| | 5-((3-(4-(trifluoromethoxy)phenyl)ureido)methyl)pyrazolo[1,5- |
| | a]pyridine-3-carboxamide |
| 57 CDD-3131 | 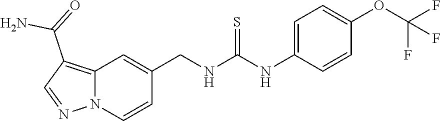 [SMILES: NC(=O)c1cnn2ccc(CNC(=S)Nc3ccc(OC(F)(F)F)cc3)cc12] |
| | 5-((3-(4-(trifluoromethoxy)phenyl)thioureido)methyl)pyrazolo[1,5- |
| | a]pyridine-3-carboxamide |
| 58 CDD-3132 | 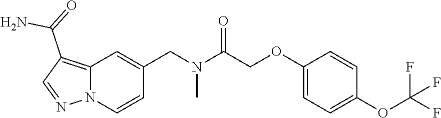 [SMILES: CN(Cc1ccn2ncc(C(N)=O)c2c1)C(=O)COc1ccc(OC(F)(F)F)cc1] |
| | 5-((N-methyl-2-(4- |
| | (trifluoromethoxy)phenoxy)acetamido)methyl)pyrazolo[1,5-a]pyridine-3- |
| | carboxamide |
| 59 CDD-3133 | 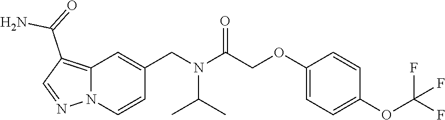 [SMILES: CC(C)N(Cc1ccn2ncc(C(N)=O)c2c1)C(=O)COc1ccc(OC(F)(F)F)cc1] |
| | 5-((N-isopropyl-2-(4- |
| | (trifluoromethoxy)phenoxy)acetamido)methyl)pyrazolo[1,5-a]pyridine-3- |
| | carboxamide |
| 60 CDD-3057 | 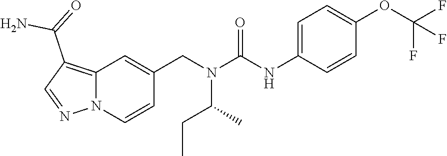 [SMILES: CC[C@@H](C)N(Cc1ccn2ncc(C(N)=O)c2c1)C(=O)Nc1ccc(OC(F)(F)F)cc1] |
| | (R)-5-((1-(sec-butyl)-3-(4- |
| | (trifluoromethoxy)phenyl)ureido)methyl)pyrazolo[1,5-a]pyridine-3- |
| | carboxamide |
| 61 CDD-3058 | 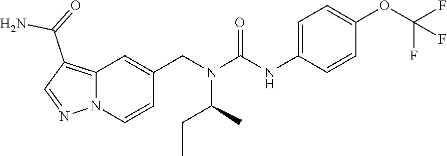 [SMILES: CC[C@H](C)N(Cc1ccn2ncc(C(N)=O)c2c1)C(=O)Nc1ccc(OC(F)(F)F)cc1] |
| | (S)-5-((1-(sec-butyl)-3-(4- |
| | (trifluoromethoxy)phenyl)ureido)methyl)pyrazolo[1,5-a]pyridine-3- |
| | carboxamide |
| 62 CDD-3181 | 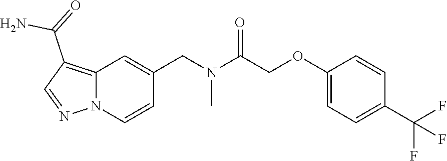 [SMILES: CN(Cc1ccn2ncc(C(N)=O)c2c1)C(=O)COc1ccc(C(F)(F)F)cc1] |
| | 5-((N-methyl-2-(4- |
| | (trifluoromethyl)phenoxy)acetamido)methyl)pyrazolo[1,5-a]pyridine-3- |
| | carboxamide |
| 63 CDD-3182 | 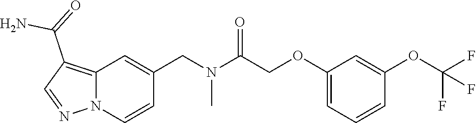 [SMILES: CN(Cc1ccn2ncc(C(N)=O)c2c1)C(=O)COc1cccc(OC(F)(F)F)c1] |
| | 5-((N-methyl-2-(3- |
| | (trifluoromethoxy)phenoxy)acetamido)methyl)pyrazolo[1,5-a]pyridine-3- |
| | carboxamide |
| 64 CDD-3183 | 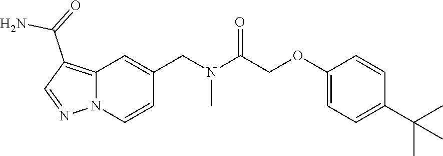 [SMILES: CN(Cc1ccn2ncc(C(N)=O)c2c1)C(=O)COc1ccc(C(C)(C)C)cc1] |
| | 5-((2-(4-(tert-butyl)phenoxy)-N-methylacetamido)methyl)pyrazolo[1,5- |
| | a]pyridine-3-carboxamide |
| 65 CDD-3184 | 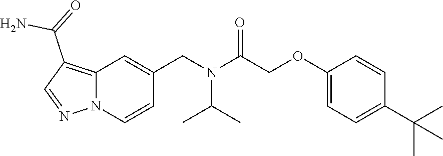 [SMILES: CC(C)N(Cc1ccn2ncc(C(N)=O)c2c1)C(=O)COc1ccc(C(C)(C)C)cc1] |
| | 5-((2-(4-(tert-butyl)phenoxy)-N-isopropylacetamido)methyl)pyrazolo[1,5- |
| | a]pyridine-3-carboxamide |
| 66 CDD-3185 | 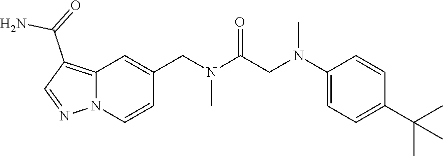 [SMILES: CN(Cc1ccn2ncc(C(N)=O)c2c1)C(=O)CN(C)c1ccc(C(C)(C)C)cc1] |
| | 5-((2-((4-(tert-butyl)phenyl)(methyl)amino)-N- |
| | methylacetamido)methyl)pyrazolo[1,5-a]pyridine-3-carboxamide |
| 67 CDD-3254 | 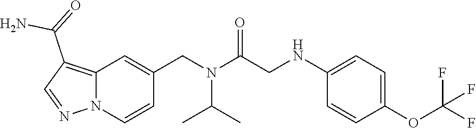 [SMILES: CC(C)N(Cc1ccn2ncc(C(N)=O)c2c1)C(=O)CNc1ccc(OC(F)(F)F)cc1] |
| | 5-((N-isopropyl-2-((4- |
| | (trifluoromethoxy)phenyl)amino)acetamido)methyl)pyrazolo[1,5- |
| | a]pyridine-3-carboxamide |
| 68 CDD-3255 | 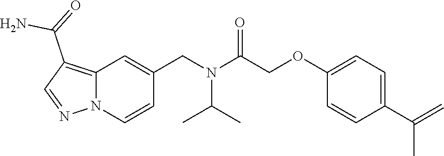 [SMILES: C=C(C)c1ccc(OCC(=O)N(Cc2ccn3ncc(C(N)=O)c3c2)C(C)C)cc1] |
| | 5-((N-isopropyl-2-(4-(prop-1-en-2- |
| | yl)phenoxy)acetamido)methyl)pyrazolo[1,5-a]pyridine-3-carboxamide |
| 69 CDD-3256 | 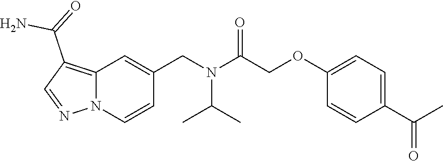 [SMILES: CC(=O)c1ccc(OCC(=O)N(Cc2ccn3ncc(C(N)=O)c3c2)C(C)C)cc1] |
| | 5-((2-(4-acetylphenoxy)-N-isopropylacetamido)methyl)pyrazolo[1,5- |
| | a]pyridine-3-carboxamide |
| 70 CDD-3257 | 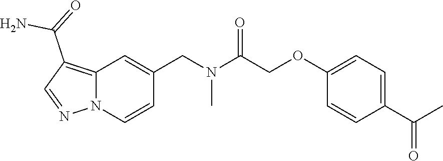 [SMILES: CC(=O)c1ccc(OCC(=O)N(C)Cc2ccn3ncc(C(N)=O)c3c2)cc1] |
| | 5-((2-(4-acetylphenoxy)-N-methylacetamido)methyl)pyrazolo[1,5- |
| | a]pyridine-3-carboxamide |
| 71 CDD-3258 | 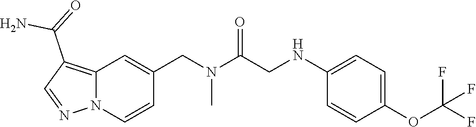 [SMILES: CN(Cc1ccn2ncc(C(N)=O)c2c1)C(=O)CNc1ccc(OC(F)(F)F)cc1] |
| | 5-((N-methyl-2-((4- |
| | (trifluoromethoxy)phenyl)amino)acetamido)methyl)pyrazolo[1,5- |
| | a]pyridine-3-carboxamide |
| 72 CDD-3099 | 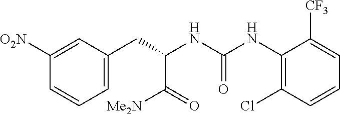 [SMILES: CN(C)C(=O)[C@H](Cc1cccc([N+](=O)[O-])c1)NC(=O)Nc1c(Cl)cccc1C(F)(F)F] |
| | (S)-2-(3-(2-chloro-6-(trifluoromethyl)phenyl)ureido)-N,N-dimethyl-3-(3- |
| | nitrophenyl)propanamide |
| 73 CDD-3100 | 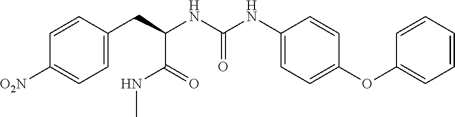 [SMILES: CNC(=O)[C@@H](Cc1ccc([N+](=O)[O-])cc1)NC(=O)Nc1ccc(Oc2ccccc2)cc1] |
| | (R)-N-methyl-3-(4-nitrophenyl)-2-(3-(4- |
| | phenoxyphenyl)ureido)propanamide |
| 74 CDD-3264 | 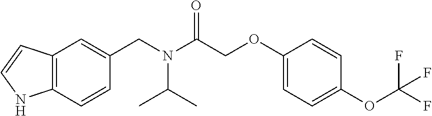 [SMILES: CC(C)N(Cc1ccc2[nH]ccc2c1)C(=O)COc1ccc(OC(F)(F)F)cc1] |
| | N-((1H-indol-5-yl)methyl)-N-isopropyl-2-(4- |
| | (trifluoromethoxy)phenoxy)acetamide |
| 75 CDD-3265 | 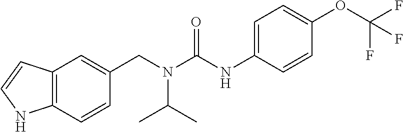 [SMILES: CC(C)N(Cc1ccc2[nH]ccc2c1)C(=O)Nc1ccc(OC(F)(F)F)cc1] |
| | 1-((1H-indol-5-yl)methyl)-1-isopropyl-3-(4- |
| | (trifluoromethoxy)phenyl)urea |
| 76 CDD-3306 | 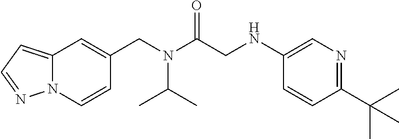 [SMILES: CC(C)N(Cc1ccn2nccc2c1)C(=O)CNc1ccc(C(C)(C)C)nc1] |
| | 2-((6-(tert-butyl)pyridin-3-yl)amino)-N-isopropyl-N-(pyrazolo[1,5- |
| | a]pyridin-5-ylmethyl)acetamide |
| 77 CDD-3307 | 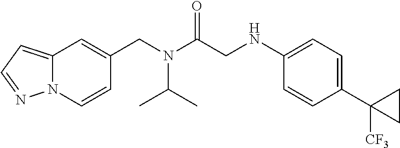 [SMILES: CC(C)N(Cc1ccn2nccc2c1)C(=O)CNc1ccc(C2(C(F)(F)F)CC2)cc1] |
| | N-isopropyl-N-(pyrazolo[1,5-a]pyridin-5-ylmethyl)-2-((4-(1- |
| | (trifluoromethyl)cyclopropyl)phenyl)amino)acetamide |
| 78 CDD-3308 | 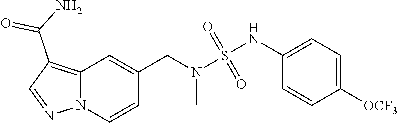 [SMILES: CN(Cc1ccn2ncc(C(N)=O)c2c1)S(=O)(=O)Nc1ccc(OC(F)(F)F)cc1] |
| | 5-((methyl(N-(4- |
| | (trifluoromethoxy)phenyl)sulfamoyl)amino)methyl)pyrazolo[1,5- |
| | a]pyridine-3-carboxamide |
| 79 CDD-2837 | 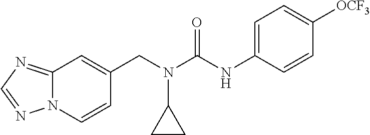 [SMILES: O=C(Nc1ccc(OC(F)(F)F)cc1)N(Cc1ccn2ncnc2c1)C1CC1] |
| | 1-([1,2,4]triazolo[1,5-a]pyridin-7-ylmethyl)-1-cyclopropyl-3-(4- |
| | (trifluoromethoxy)phenyl)urea |
| 80 CDD-2838 | 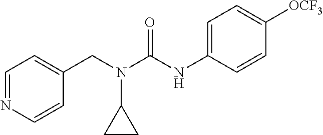 [SMILES: O=C(Nc1ccc(OC(F)(F)F)cc1)N(Cc1ccncc1)C1CC1] |
| | 1-cyclopropyl-1-(pyridin-4-ylmethyl)-3-(4-(trifluoromethoxy)phenyl)urea |
| 81 CDD-2839 | 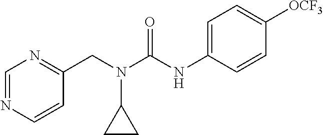 [SMILES: O=C(Nc1ccc(OC(F)(F)F)cc1)N(Cc1ccncn1)C1CC1] |
| | 1-cyclopropyl-1-(pyrimidin-4-ylmethyl)-3-(4- |
| | (trifluoromethoxy)phenyl)urea |
| 82 CDD-2840 | 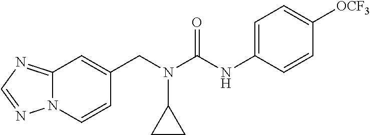 [SMILES: O=C(Nc1ccc(OC(F)(F)F)cc1)N(Cc1ccn2ncnc2c1)C1CC1] |
| | 1-cyclopropyl-1-(imidazo[1,2-a]pyridin-7-ylmethyl)-3-(4- |
| | (trifluoromethoxy)phenyl)urea |
| 83 CDD-3033 | 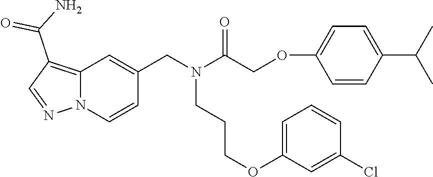 [SMILES: CC(C)c1ccc(OCC(=O)N(CCCOc2cccc(Cl)c2)Cc2ccn3ncc(C(N)=O)c3c2)cc1] |
| | 5-((N-(3-(3-chlorophenoxy)propyl)-2-(4- |
| | isopropylphenoxy)acetamido)methyl)pyrazolo[1,5-a]pyridine-3- |
| | carboxamide |
| 84 CDD-3222 | 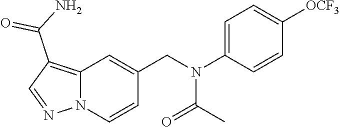 [SMILES: CC(=O)N(Cc1ccn2ncc(C(N)=O)c2c1)c1ccc(OC(F)(F)F)cc1] |
| | 5-((N-(4-(trifluoromethoxy)phenyl)acetamido)methyl)pyrazolo[1,5- |
| | a]pyridine--3-carboxamide |
| 85 CDD-3223 | 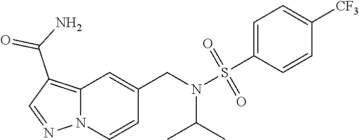 [SMILES: CC(C)N(Cc1ccn2ncc(C(N)=O)c2c1)S(=O)(=O)c1ccc(C(F)(F)F)cc1] |
| | 5-(((N-isopropyl-4- |
| | (trifluoromethyl)phenyl)sulfonamido)methyl)pyrazolo[1,5-a]pyridine-3- |
| | carboxamide |
| 86 CDD-3224 | 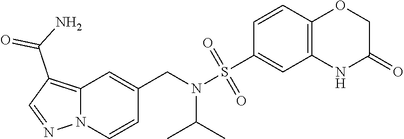 [SMILES: CC(C)N(Cc1ccn2ncc(C(N)=O)c2c1)S(=O)(=O)c1ccc2c(c1)NC(=O)CO2] |
| | 5-(((N-isopropyl-3-oxo-3,4-dihydro-2H-benzo[b][1,4]oxazine)-6- |
| | sulfonamido)methyl)pyrazolo[1,5-a]pyridine-3-carboxamide |
| 87 CDD-3225 | 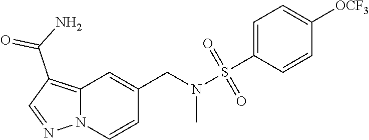 [SMILES: CN(Cc1ccn2ncc(C(N)=O)c2c1)S(=O)(=O)c1ccc(OC(F)(F)F)cc1] |
| | 5-(((N-methyl-4- |
| | (trifluoromethoxy)phenyl)sulfonamido)methyl)pyrazolo[1,5-a]pyridine-3- |
| | carboxamide |
| 88 CDD-3226 | 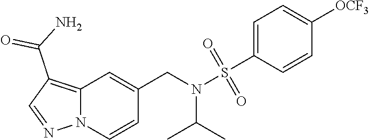 [SMILES: CC(C)N(Cc1ccn2ncc(C(N)=O)c2c1)S(=O)(=O)c1ccc(OC(F)(F)F)cc1] |
| | 5-(((N-isopropyl-4- |
| | (trifluoromethoxy)phenyl)sulfonamido)methyl)pyrazolo[1,5-a]pyridine-3- |
| | carboxamide |
| 89 | 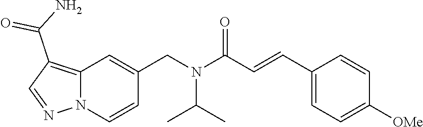 [SMILES: COc1ccc(/C=C/C(=O)N(Cc2ccn3ncc(C(N)=O)c3c2)C(C)C)cc1] |
| | (E)-5-((N-isopropyl-3-(4- |
| | methoxyphenyl)acrylamido)methyl)pyrazolo[1,5-a]pyridine-3- |
| | carboxamide |
| 90 | 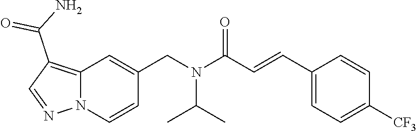 [SMILES: CC(C)N(Cc1ccn2ncc(C(N)=O)c2c1)C(=O)/C=C/c1ccc(C(F)(F)F)cc1] |
| | (E)-5-((N-isopropyl-3-(4- |
| | (trifluoromethyl)phenyl)acrylamido)methyl)pyrazolo[1,5-a]pyridine-3- |
| | carboxamide |

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means.

Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate anine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), N-alkoxycarbonyl (Alloc), methyl (Me), ethyl (Et), tert-butyl (t-Bu), tert-butyl dimethylsilyl (TBDMS), 2-(trimethylsilyl)ethoxycarbonyl (Teoc), tert-butyloxycarbonyl (Boc), para-methoxybenzyl (PMB), triphenylmethyl (trityl), fluorenylmethyloxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

In one aspect, the present disclosure provides a pharmaceutical composition comprising the compound of the present disclosure and a pharmaceutically acceptable carrier.

The compositions containing the compound(s) described herein include a pharmaceutical composition comprising at least one compound as described herein and at least one pharmaceutically acceptable carrier. In some embodiments, the pharmaceutical composition comprises Kolliphor EL, and aqueous buffer, or a combination thereof. In certain embodiments, the aqueous buffer comprises phosphate buffered saline (PBS). In some embodiments, the aqueous buffer comprises 1× PBS. In certain embodiments, the pharmaceutical composition comprises about 5%, about 10%, about 15%, about 20%, about 25%, about 30%, about 35%, or about 40% Kolliphor EL. In some embodiments, the pharmaceutical composition comprises about 20% Kolliphor EL in 1× PBS.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. In some embodiments, the composition is formulated as a pill, tablet, gelcap, or capsule for oral administration.

### Methods

In one aspect, the present disclosure provides a method of treating, preventing, and/or ameliorating endometriosis in a subject in need thereof. In certain embodiments, the method comprises administering to the subject a Neurotrophic Receptor Tyrosine Kinase (NTRK) inhibitor or a pharmaceutical composition comprising the NTRK inhibitor and a pharmaceutically acceptable carrier.

In certain embodiments, the NTRK inhibitor is a compound of the present disclosure.

In another aspect, the present disclosure provides a method of treating, preventing, and/or ameliorating endometriosis in a subject in need thereof, the method comprising administering to the subject a compound of the present disclosure and/or a pharmaceutical composition of the present disclosure.

In certain embodiments, at least one selected from the group consisting of chronic pelvic pain, inflammation, and infertility is treated, prevented, and/or ameliorated in the subject.

In certain embodiments, the subject is a female.

In another aspect, the present disclosure provides a method of treating, preventing, and/or ameliorating cancer in a subject in need thereof. In certain embodiments, the method comprises administering to the subject a Neurotrophic Receptor Tyrosine Kinase (NTRK) inhibitor or a pharmaceutical composition comprising the NTRK inhibitor and a pharmaceutically acceptable carrier.

In certain embodiments, the NTRK inhibitor is a compound of the present disclosure.

In another aspect, the present disclosure provides a method of treating, preventing, and/or ameliorating cancer in a subject in need thereof, the method comprising administering to the subject a compound of the present disclosure and/or a pharmaceutical composition of the present disclosure.

In another aspect, the present disclosure provides a method of treating, preventing, and/or ameliorating pain in a subject in need thereof. In certain embodiments, the method comprises administering to the subject a Neurotrophic Receptor Tyrosine Kinase (NTRK) inhibitor or a pharmaceutical composition comprising the NTRK inhibitor and a pharmaceutically acceptable carrier.

In certain embodiments, the NTRK inhibitor is a compound of the present disclosure.

In another aspect, the present disclosure provides a method of treating, preventing, and/or ameliorating pain in a subject in need thereof, the method comprising administering to the subject a compound of the present disclosure and/or a pharmaceutical composition of the present disclosure.

In certain embodiments, neurotrophic receptor tyrosine kinase 1 (NTRK1) is overexpressed in the subject. In certain embodiments, neurotrophic receptor tyrosine kinase 2 (NTRK2) is overexpressed in the subject. In certain embodiments, neurotrophic receptor tyrosine kinase 3 (NTRK3) is overexpressed in the subject.

In certain embodiments, administration of the compound of the present disclosure and/or the pharmaceutical composition of the present disclosure results in inhibition of NTRK1 in the subject. In certain embodiments, inhibition of NTRK1 results in treatment, prevention, and/or amelioration of endometriosis, and/or one or more symptoms thereof, cancer, and/or pain.

In certain embodiments, administration of the compound of the present disclosure and/or the pharmaceutical composition of the present disclosure results in inhibition of NTRK2 in the subject. In certain embodiments, inhibition of NTRK2 results in treatment, prevention, and/or amelioration of endometriosis, and/or one or more symptoms thereof, cancer, and/or pain.

In certain embodiments, administration of the compound of the present disclosure and/or the pharmaceutical composition of the present disclosure results in inhibition of NTRK3 in the subject. In certain embodiments, inhibition of NTRK3 results in treatment, prevention, and/or amelioration of endometriosis, and/or one or more symptoms thereof, cancer, and/or pain.

In certain embodiments, a fusion gene comprising at least one selected from the group consisting of NTRK1, NTRK2, and NTRK3 is overexpressed in the subject.

In certain embodiments, the NTRK inhibitor is a NTRK2 inhibitor.

In certain embodiments, the NTRK2 inhibitor selectively inhibits NTRK2 over NTRK11. In certain embodiments, the NTRK2 inhibitor selectively inhibits NTRK2 over NTRK3. In certain embodiments, the NTRK2 inhibitor selectively inhibits NTRK2 over NTRK1 and NTRK3.

In certain embodiments, selectivity of a compound for NTRK2 over NTRK1 comprises a half-maximal inhibitory concentration (IC₅₀) of the compound for NTRK2 which is at least 2, 3, 4, 5, 6, 7, 8, 9, 10, 20, 50, 100, or 1000 times lower than the IC₅₀ for the same compound against NTRK1. In certain embodiments, selectivity of a compound for NTRK2 over NTRK1 comprises a half-maximal inhibitory concentration (IC₅₀) of the compound for NTRK2 which is at least 2, 3, 4, 5, 6, 7, 8, 9, 10, 20, 50, 100, or 1000 times lower than the IC₅₀ for the same compound against NTRK3.

In certain embodiments, the NTRK2 inhibitor is staurosporine. In certain embodiments, the NTRK2 inhibitor is entrectinib. In certain embodiments, the NTRK2 inhibitor is larotrectinib. In certain embodiments, the NTRK2 inhibitor is selitrectinib. In certain embodiments, the NTRK2 inhibitor is reprotrectinib. In certain embodiments, the NTRK2 inhibitor is PF-06273340.

In certain embodiments, administration of the compound of the present disclosure and/or the pharmaceutical composition of the present disclosure results in inhibition of the fusion gene product in the subject. In certain embodiments, inhibition of the fusion gene product results in treatment, prevention, and/or amelioration of endometriosis, and/or one or more symptoms thereof, cancer, and/or pain.

In certain embodiments, the subject is a mammal. In certain embodiments, the mammal is a human.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. To maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Materials and Methods

### Materials and Instrumentation

All reactions involving air-sensitive reagents were carried out in anhydrous solvents under an atmosphere of nitrogen. Reagents and solvents purchased from commercial supplies were used as received. Reactions were monitored by thin-layer chromatography (TLC) on BAKER-FLEX® silica gel plates (IB2-F) using UV-light (254 and 365 nm) detection or high-performance liquid chromatography/mass spectrometry (HPLC-MS). Column chromatography was carried out using Teledyne ISCO CombiFlash system equipped with either a silica or C-18 column. NMR spectra were recorded at room temperature using a Bruker Avance III HD 600 MHz spectrometer (¹H NMR at 600 MHz). Chemical shifts (δ) are reported in parts per million (ppm) with reference to solvent signals [¹H-NMR: DMSO-d₆ (2.50 ppm) and CD₃Cl₃ (7.26 ppm)]. Signal patterns are reported as s (singlet), d (doublet), dd (double doublet), t (triplet), q (quartet), m (multiplet), and br (broad). Coupling constants (J) are given in Hz. HRMS measurements were performed using ThermoFisher Scientific Q Exactive instrument. Abbreviations presented in experimental procedures are referred to the following definitions: NaBH(OAc)₃, Sodium triacetoxyhydroborate; CHCl₃, chloroform; DCM, dichloromethane; CH₃CN, acetonitrile; MeOH, methanol; NaHCO₃, sodium bicarbonate, DMF, N,N-dimethylformamide; EtOAc, ethyl acetate; HATU, 0-(7-azabenzotriazol-1-yl)-N,N,N′,N′-tetramethyluronium hexafluorophosphate; LiOH, lithium hydroxide; NaOH_{(aq)}; TEA, triethylamine; THF, tetrahydrofuran; HCl, hydrochloric acid.

### Example 1: Novel Kinases as Therapeutic Targets in Endometriosis

Balanced phosphorylation activity mediated by kinases and phosphatases encoded within the human genome is critical for controlling the intracellular response to internal and external cues. This balanced kinase and phosphatase activity is critical for maintaining cellular function and preventing dysregulated cellular growth. Identifying inhibitors for overactive kinases and phosphatases in cancer is a large area of interest within the academic and industry drug discovery community. There are approximately 538 kinases and 200 phosphatases in the human genome, with 73 currently-available FDA-approved kinase inhibitors. In addition to the 73 FDA-approved kinase inhibitors that target 42 different primary kinases, there are several hundred ongoing clinical trials for another 15-20 protein kinases. Therefore, only ˜70 kinases of the known 538 kinases are currently being targeted for drug inhibition, indicating that a vast number of therapeutic opportunities exist by targeting kinase domains. Further, these FDA-approved kinase inhibitors are often promiscuous, inhibiting multiple related kinases. For example, Entrectinib, a pan-NTRK inhibitor, is a potent 7 nM inhibitor of ROS1 and a 12 nM inhibitor of anaplastic lymphoma kinase (ALK); Pexidartinib inhibits FLT3-ITD, KIT, and CSFIR with IC₅₀ values of 9, 12, and 17 nM, respectively; and Ripretinib targets KIT, PDRFα, BRAF, and DDR2 in the 3-7 nM IC₅₀ range.

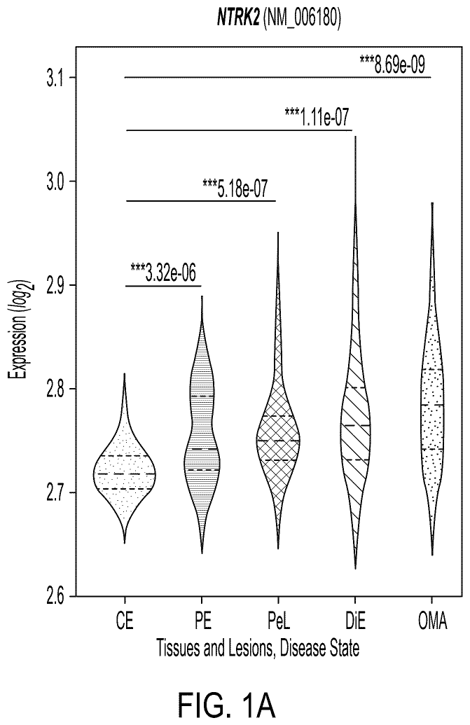

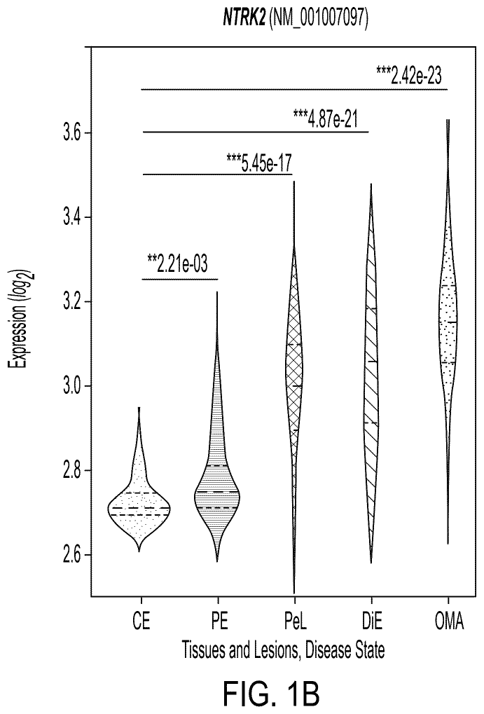

To identify overexpressed kinases in endometriosis, a comprehensive database was analyzed which compares endometrial biopsies from 53 controls versus endometriosis subtype lesions (i.e., peritoneal lesion (PeL), ovarian endometrioma (OMA), and deep infiltrating endometriosis (DiE)) from 115 patients. To identify differentially expressed kinases, the normalized microarray values were analyzed to identify kinase-encoding genes that were upregulated (>1.4-fold, p<0.05), and chosen for high expression across all three subtypes. From this analysis, 20 kinases were identified for validation and therapeutic targeting in endometriosis (FIGS. 1A-1B).

### Example 2: Identification of NTRK2 as a Target Kinase for Endometriosis

NTRK2 was among the kinases identified in the study described herein, which is a target of an FDA-approved small molecule inhibitor (i.e., entrectinib). Thus, NTRK2 represents a kinase target with a corresponding FDA-approved inhibitor that can be evaluated as a tool compound for endometriosis.

NTRK1, NTRK2, and NTRK3 are tyrosine kinase receptors for their extracellular ligands nerve growth factor (NGF), brain derived nerve growth factor (BDNF) and neurotrophin 4 (NT-4), and neurotrophin 3 (NT-3), respectively. Further, previous data has indicated the risk of on-target adverse events associated with both NTRK1 and NTRK2 resulting from pharmacologically relevant exposure in the CNS. Therefore, developing a safer, peripherally restricted NTRK2 inhibitor must be considered for this non-life-threatening indication.

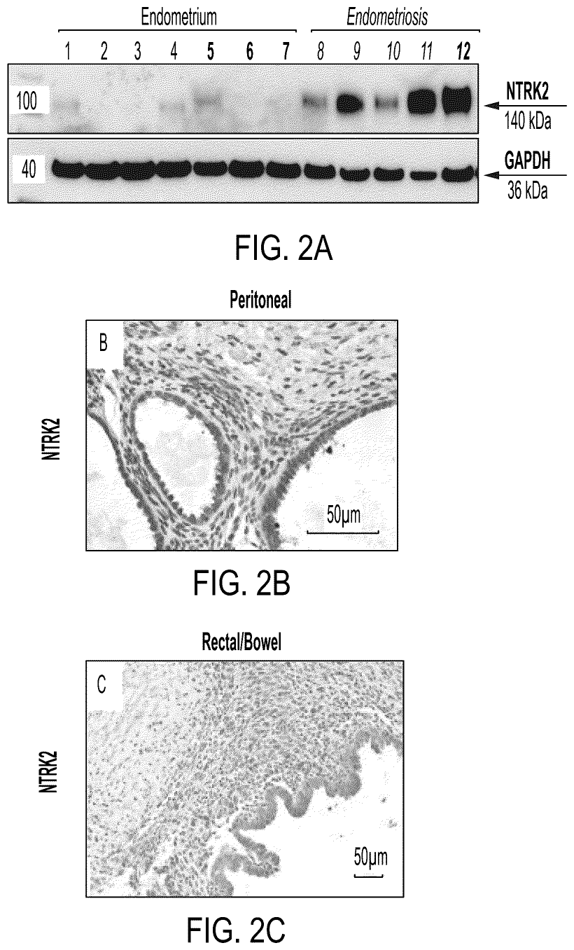

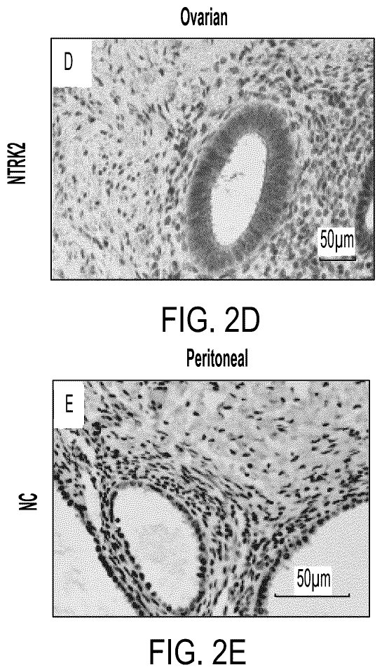

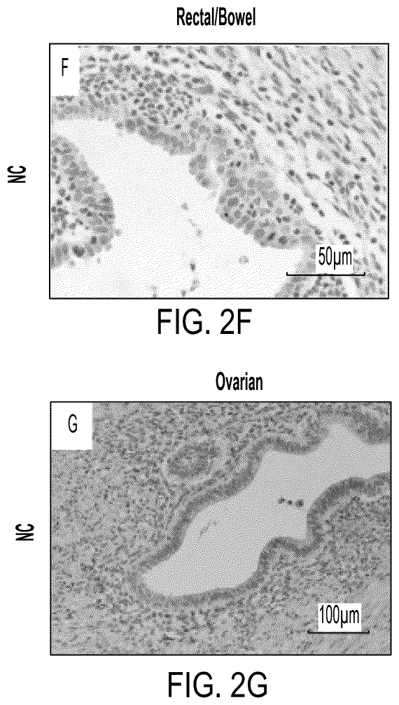

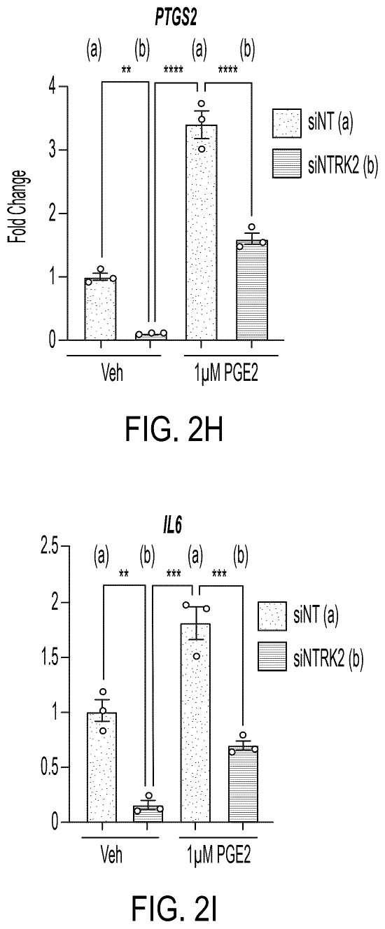

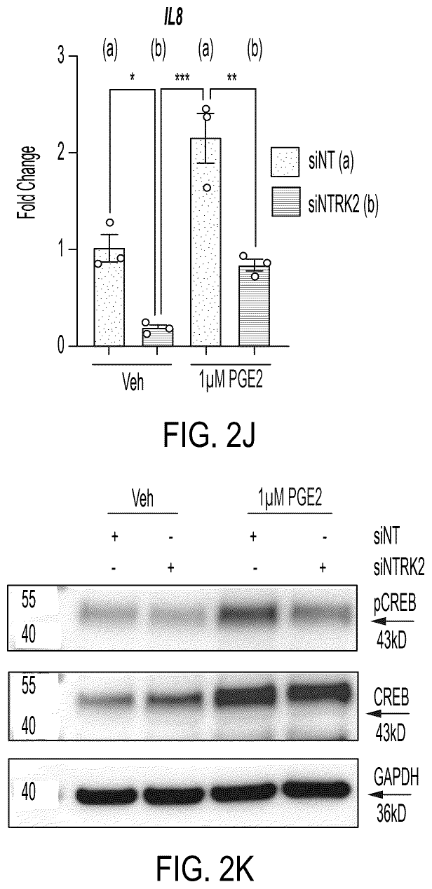

Using Western blot analysis, it was discovered that NTRK2 protein levels are increased in endometriotic tissue versus eutopic tissue (FIG. 2A). NTRK2 showed elevated expression in lysates from ectopic peritoneal lesions and ovarian endometriomas as compared to a eutopic normal endometrium (FIGS. 2B-2G). It was also determined that siRNA-mediated knockdown of NTRK2 decreased prostaglandin E2 (PGE2)-mediated induction of pro-inflammatory genes (PTGS2/COX2, IL6, IL8) in primary endometriotic stromal cells from an endometrioma (FIGS. 2H-2J). Western blot analysis showed that this NTRK2-mediated decrease in pro-inflammatory genes also decreased phosphorylated cAMP-response element binding protein (CREB) (FIG. 2K).

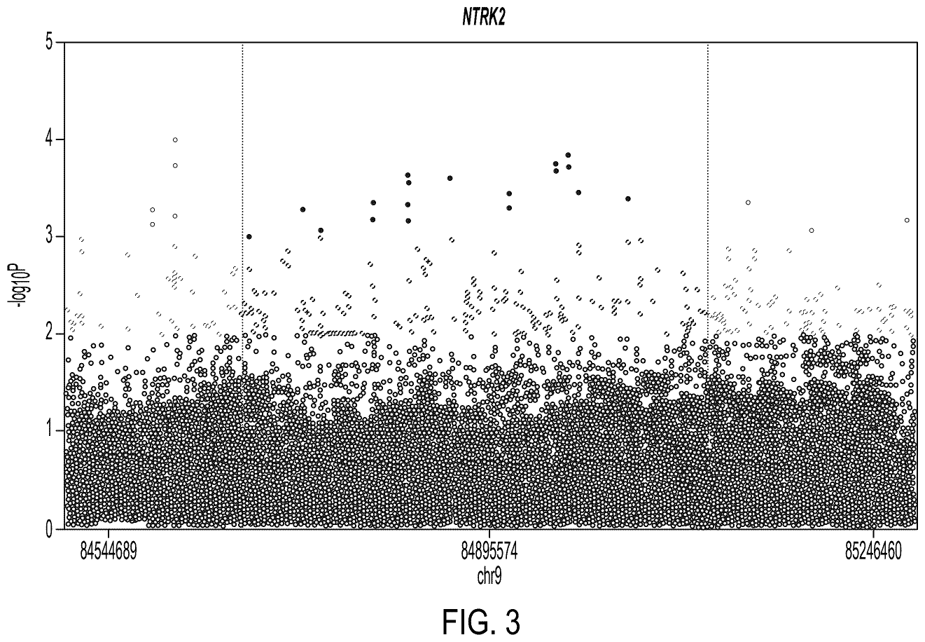

Further, analysis of 36,697 endometriosis cases and 116,071 controls in the FinnGen database identified the association of 20 (p<0.001) NTRK2 variants with various subtypes and stages of endometriosis (FIG. 3).

### Example 3: Identification of NTRK2 Inhibitors

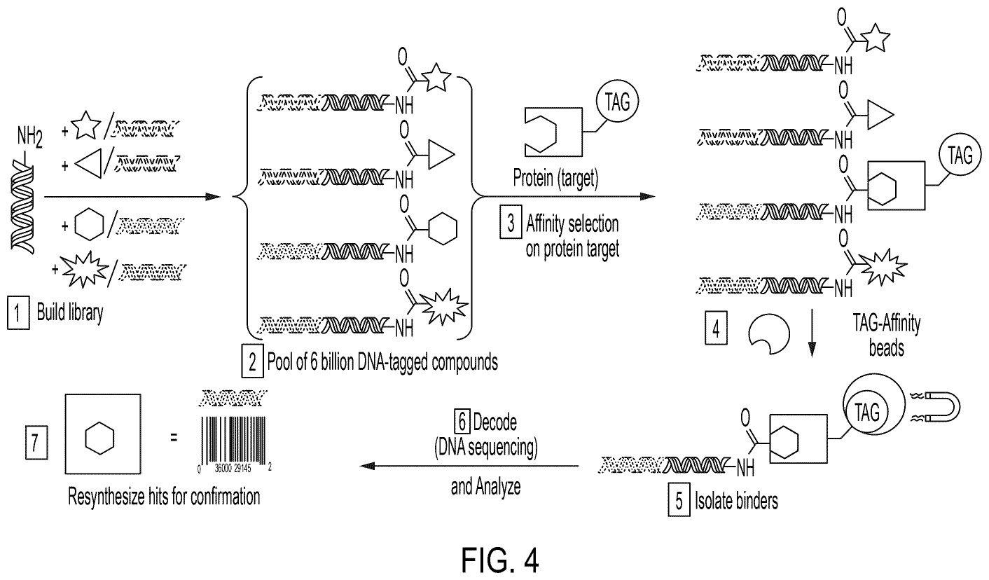

To identify NTRK2 selective inhibitors, a DNA-Encoded Chemistry Technology (DEC-Tec) strategy was employed. DEC-Tec is a platform for the identification of drug-like molecules with high affinity binding to target proteins. DEC-Tec platforms allow the exploration of chemical space at a greater level than traditional high-throughput screening methods. By using “split and pool” combinatorial synthesis techniques, DEC-Tec can effectively generate screening libraries with up to 109 uniquely encoded chemical compounds (FIG. 4).

Parallel selections were performed against 4.25 billion DNA-encoded small molecules with NTRK2, without NTRK2 (i.e., control), or in the presence of the promiscuous kinase inhibitor, staurosporine. These selections included kinase-biased molecules. Enrichment of each library member, measured by a normalized z-score, from the selections were compared.

A series of compounds were identified that were significantly enriched in the NTRK2-only selection, but not in the control or NTRK2 plus staurosporine selections, consistent with competitive ligand binding.

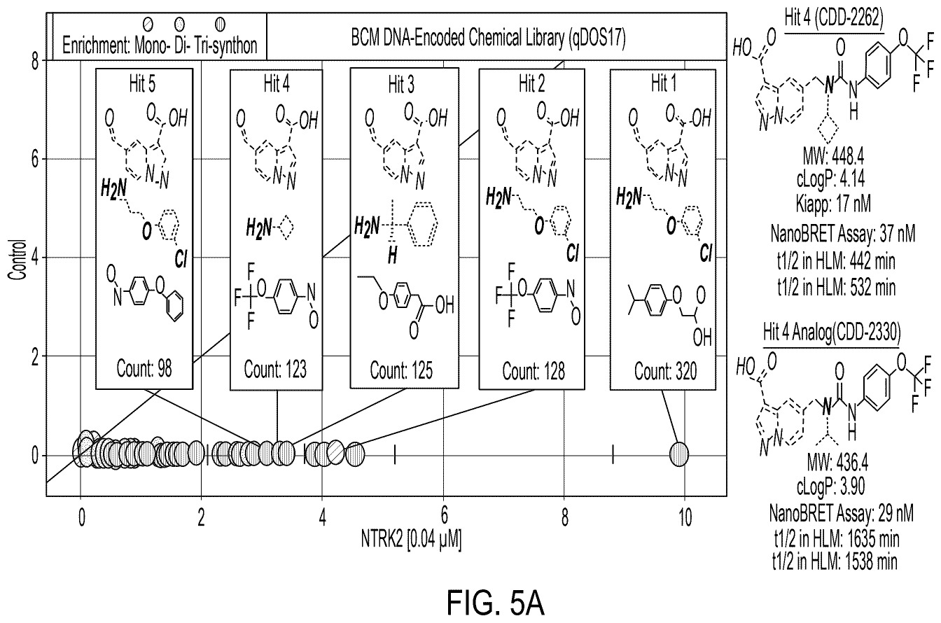

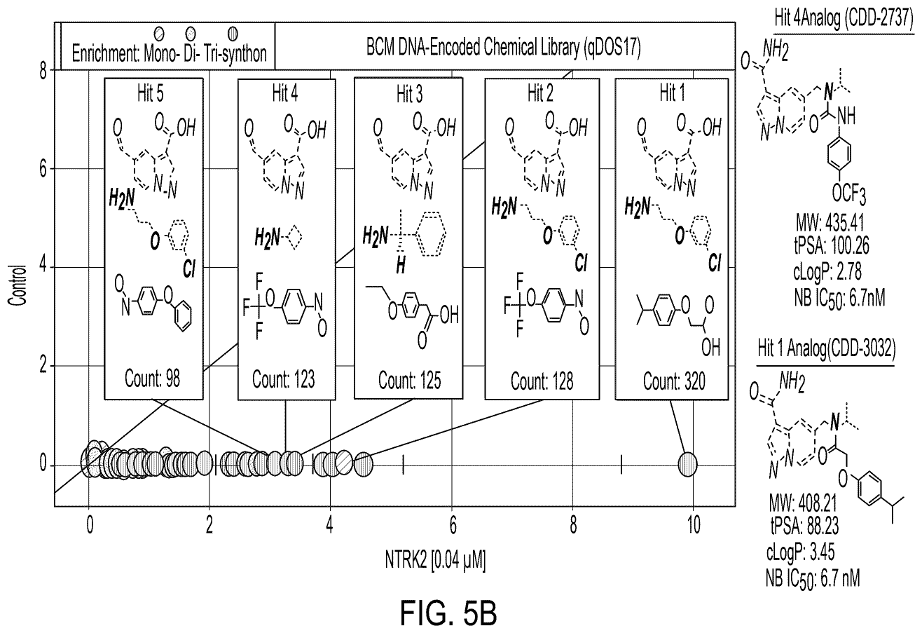

Because this NTRK2 hit series had many drug-like characteristics, several tri-synthon hits and analogs were selected for synthesis off-DNA for in vitro testing, including CDD-2262, and its smaller analog, CDD-2330 (FIG. 5).

### Example 4: Compound Synthesis

### General Procedure I: Reductive Amination

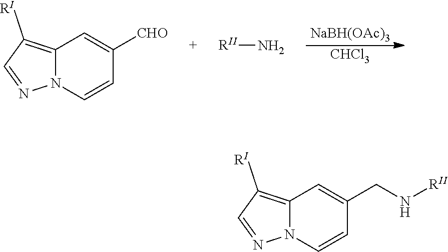

An oven-dried microwave vial, which was equipped with a magnetic stir bar, was charged with aryl aldehyde (1.0 equiv.), primary amine (2.0 equiv.), and CHCl₃ (0.2-0.4 M). The solution was stirred at room temperature for at least 2 hours, then NaBH(OAc)₃ (1.5 equiv.) was added in portions. After overnight, additional NaBH(OAc)₃ (1.5 equiv.) was added. After 4 hours, the reaction mixture was quenched by adding NaOH (1.0 M) until pH=9. The aqueous layer was extracted by DCM/MeOH (9:1) three times. The combined organic layers were concentrated and the residual was separated by chromatography to afford desired secondary amine product. Compounds 13-15 and 27 were prepared according to General Procedure I.

### General Procedure H: Urea Synthesis

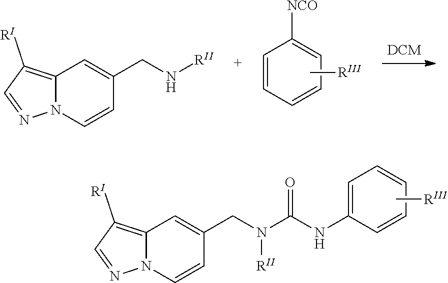

A secondary amine (1.0 equiv.) was dissolved in DCM (0.2 M), then an isocyanate (2.0 equiv.) was added. The reaction was allowed to proceed overnight, then the solution was concentrated, and the crude product was purified by chromatography to afford the desired urea product. In certain embodiments, the secondary amine may be obtained as described in General Procedure I. Compounds 2-11, 16-19, 21-23, and 25-26 were prepared according to General Procedure II.

### General Procedure III: Amide Coupling

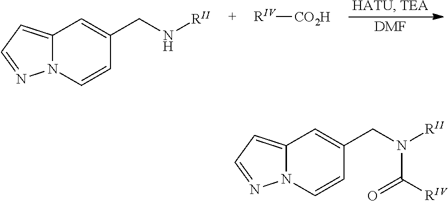

A carboxylic acid (1.0 equiv.) was dissolved in DMF (0.2 M), then HATU (1.2 equiv.) was added. After stirring at room temperature for 5 minutes, a secondary amine (1.3 equiv.) was added, followed by addition of Et₃N (1.8 equiv.). The reaction was allowed to proceed overnight, was quenched with saturated NaHCO₃, and was extracted three times with EtOAc. Evaporation of the combined organic layers provided the crude product which was purified by chromatography to afford desired amide product. In certain embodiments, the secondary amine may be obtained as described in General Procedure T. Compounds 12 and 24-25 were prepared according to General Procedure III.

### General Procedure IV. Ester Hydrolysis

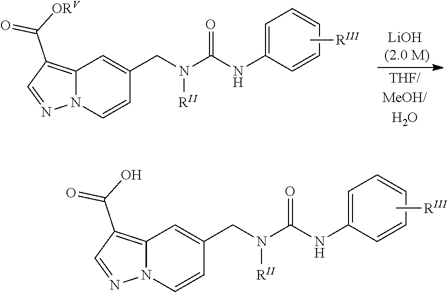

A methyl ester was dissolved in a solvent mixture comprising THF/MeOH/H₂O (4:1:1, 0.2 M), then an aqueous solution of LiOH (2.0 M, 5 equiv.) was added dropwise at room temperature. The reaction proceeded for 4 h, then additional aq. LiOH was added. The reaction proceeded overnight and was subsequently quenched with 1M HCl, and the pH was adjusted to pH=2-3. The mixture was concentrated to provide the crude product, which was was purified by C-18 column chromatography to afford the desired carboxylic acid product. Compounds 1 and 21 were prepared according to General Procedure IV.

### General Procedure V: Amide Coupling

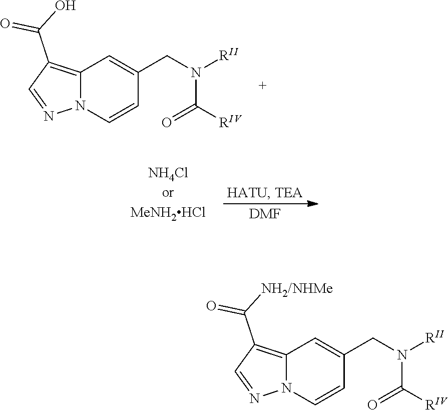

A carboxylic acid (1.0 equiv.) was dissolved in DMF (0.2 M), then HATU (1.2 equiv.) was added. After stirring at room temperature for 5 minutes, ammonium chloride or methylamine salt (1.3 equiv.) was added, followed by addition of Et₃N (1.8 equiv.). The reaction was allowed to proceed overnight, quenched with saturated NaHCO₃, and extracted three times with EtOAc. Evaporation of the combined organic layers provided the crude product which was purified by chromatography to afford desired amide product.

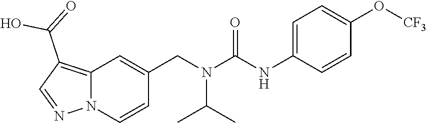 [SMILES: CC(C)N(Cc1ccn2ncc(C(=O)O)c2c1)C(=O)Nc1ccc(OC(F)(F)F)cc1]

### 5-((1-isopropyl-3-(4-(trifluoromethoxy)phenyl)ureido)methyl)pyrazolo[1,5-a]pyridine-4-carboxylic acid (1)

¹H NMR (600 MHz, CDCl₃) δ (ppm) 8.46 (d, J=6.6 Hz, 1H), 8.41 (s, 1H), 8.08 (s, 1H), 7.34 (d, J=9.0 Hz, 2H), 7.08 (d, J=8.4 Hz, 2H), 6.96 (d, J=6.0 Hz, 2H), 6.69 (s, 1H), 4.56 (s, 2H), 4.45 (t, J=6.0 Hz, 3H), 1.22 (d, J=6.0, 6H); ¹³C NMR (600 MHz, CDCl₃) δ (ppm) 168.3, 155.6, 146.0, 141.1, 137.5, 129.5, 121.7, 121.6, 119.6, 115.7, 113.1, 103.0, 53.4, 47.8, 45.0, 20.9; HRMS (ESI) m/z calcd for C₂₀H₁₉F₃N₄O₄ [M+H]⁺ 437.1358, found 437.1421.

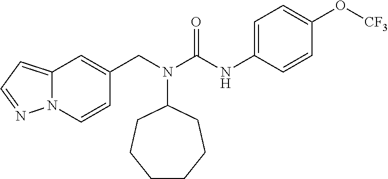 [SMILES: O=C(Nc1ccc(OC(F)(F)F)cc1)N(Cc1ccn2nccc2c1)C1CCCCCC1]

### 1-cycloheptyl-1-(pyrazolo[1,5-a]pyridin-5-ylmethyl)-3-(4-(trifluoromethoxy)phenyl)urea (2)

¹H NMR (600 MHz, CDCl₃) δ (ppm) 8.44 (d, J=7.2 Hz, 1H), 7.96 (s, 1H), 7.49 (s, 1H), 7.27 (d, J=9.0 Hz, 2H), 7.08 (d, J=8.4 Hz, 2H), 6.68 (dd, J=7.2 Hz, 1H), 6.49 (d, J=1.8 Hz, 1H), 6.32 (s, 1H), 4.50 (s, 2H), 4.25 (m, 1H), 1.96 (m, 2H), 1.72 (m, 2H), 1.65 (m, 4H), 1.51 (m, 4H).

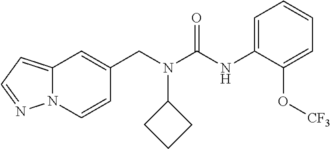 [SMILES: O=C(Nc1ccccc1OC(F)(F)F)N(Cc1ccn2nccc2c1)C1CCC1]

### 1-cyclobutyl-1-(pyrazolo[1,5-a]pyridin-5-ylmethyl)-3-(2-(trifluoromethoxy)phenyl)urea (3)

¹H NMR (600 MHz, CDCl₃) δ (ppm) 8.43 (d, J=7.2 Hz, 1H), 8.29 (d, J=7.2 Hz, 1H), 7.93 (d, J=1.8 Hz, 1H), 7.40 (s, 1H), 7.25 (m, 1H), 7.18 (m, 1H), 7.01 (m, 1H), 6.80 (s, 1H), 6.68 (dd, J=7.2 Hz, 1H), 6.45 (d, J=1.8 Hz, 1H), 4.64 (s, 2H), 4.45 (m, 1H), 2.32 (m, 2H), 2.18 (m, 2H), 1.75 (m, 2H).

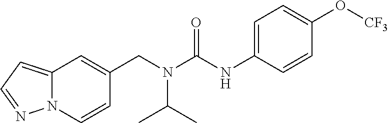 [SMILES: CC(C)N(Cc1ccn2nccc2c1)C(=O)Nc1ccc(OC(F)(F)F)cc1]

### 1-isopropyl-1-(pyrazolo[1,5-a]pyridin-5-ylmethyl)-3-(4-(trifluoromethoxy)phenyl)urea (4)

¹H NMR (600 MHz, CDCl₃) δ (ppm) 8.46 (d, J=7.2 Hz, 1H), 7.97 (d, J=1.8 Hz, 1H), 7.50 (s, 1H), 7.28 (d, J=9.0 Hz, 2H), 7.09 (d, J=9.0 Hz, 2H), 6.70 (dd, J=7.2 Hz, 1H), 6.50 (d, J=1.8 Hz, 1H), 6.33 (s, 1H), 4.63 (m, 1H), 4.48 (s, 2H), 1.25 (d, J=6.6 Hz, 6H); ¹³C NMR (600 MHz, CDCl₃) δ (ppm) 155.4, 144.6, 142.6, 139.9, 137.6, 134.3, 129.1, 121.6, 121.1, 114.7, 110.4, 97.1, 47.1, 44.9, 20.8; HRMS (ESI) m/z calcd for C₁₉H₁₉F₃N₄O₂ [M+H]⁺ 393.1460, found 393.1533.

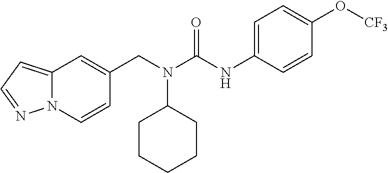 [SMILES: O=C(Nc1ccc(OC(F)(F)F)cc1)N(Cc1ccn2nccc2c1)C1CCCCC1]

### 1-cyclohexyl-1-(pyrazolo[1,5-a]pyridin-5-ylmethyl)-3-(4-(trifluoromethoxy)phenyl)urea (5)

¹H NMR (600 MHz, CDCl₃) δ (ppm) 8.43 (d, J=7.2 Hz, 1H), 7.96 (d, J=2.4 Hz, 1H), 7.49 (s, 1H), 7.27 (d, J=9.0 Hz, 2H), 7.08 (d, J=9.0 Hz, 2H), 6.67 (dd, J=7.2 Hz, 1H), 6.48 (d, J=1.8 Hz, 1H), 6.35 (s, 1H), 4.50 (s, 2H), 4.17 (m, 1H), 1.89 (m, 2H), 1.82 (m, 2H), 1.68 (m, 1H), 1.39 (m, 4H), 1.098 (m, 1H). HRMS (ESI) m/z calcd for C₂₂H₂₃F₃N₄O₂ [M+H]⁺ 433.1773, found 433.1842.

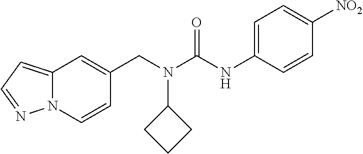 [SMILES: O=C(Nc1ccc([N+](=O)[O-])cc1)N(Cc1ccn2nccc2c1)C1CCC1]

### 1-cyclobutyl-3-(4-nitrophenyl)-1-(pyrazolo[1,5-a]pyridin-5-ylmethyl)urea (6)

¹H NMR (600 MHz, CDCl₃) δ (ppm) 8.37 (d, J=7.2 Hz, 1H), 8.09 (d, J=6.0 Hz, 2H), 7.88 (d, J=2.4, 1H), 7.50 (d, J=9.0 Hz, 2H), 7.35 (s, 1H), 7.17 (s, 1H), 6.63 (dd, J=7.2 Hz, 1H), 6.43 (d, J=1.8 Hz, 1H), 4.61 (s, 2H), 4.43 (m, 1H), 2.25 (m, 2H), 2.11 (m, 2H), 1.68 (m, 2H).

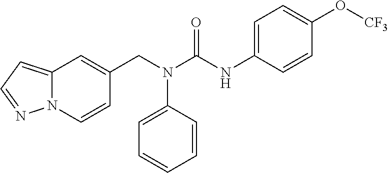 [SMILES: O=C(Nc1ccc(OC(F)(F)F)cc1)N(Cc1ccn2nccc2c1)c1ccccc1]

### 1-phenyl-1-(pyrazolo[1,5-a]pyridin-5-ylmethyl)-3-(4-(trifluoromethoxy)phenyl)urea (7)

¹H NMR (600 MHz, CDCl₃) δ (ppm) 8.40 (d, J=7.2 Hz, 1H), 7.91 (d, J=1.8 Hz, 1H), 7.43 (m, 2H), 7.38 (m, 1H), 7.33 (s, 1H), 7.31 (m, 2H), 7.21 (d, J=7.2 Hz, 2H), 7.11 (d, J=8.4 Hz, 2H), 6.84 (dd, J=7.2 Hz, 1H), 6.41 (d, J=1.8 Hz, 1H), 6.24 (s, 1H), 4.93 (s, 2H).

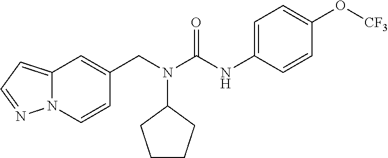 [SMILES: O=C(Nc1ccc(OC(F)(F)F)cc1)N(Cc1ccn2nccc2c1)C1CCCC1]

### 1-cyclopentyl-1-(pyrazolo[1,5-a]pyridin-5-ylmethyl)-3-(4-(trifluoromethoxy)phenyl)urea (8)

¹H NMR (600 MHz, CDCl₃) δ (ppm) 8.45 (d, J=7.2 Hz, 1H), 7.95 (d, J=1.8 Hz, 1H), 7.46 (s, 1H), 7.29 (d, 9.0 Hz, 2H), 7.09 (d, J=8.4 Hz, 2H), 6.67 (dd, J=7.2 Hz, 1H), 6.48 (d, J=1.8 Hz, 1H), 6.34 (s, 1H), 4.62 (m, 1H), 4.49 (s, 2H), 2.00 (m, 2H), 1.72 (m, 2H), 1.64 (m, 2H), 1.61 (m, 2H), 1.53 (m, 2H). HRMS (ESI) m/z calcd for C₂₁H₂₁F₃N₄O₂ [M+H]419.1617, found 419.1669.

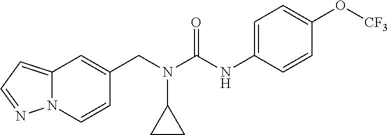 [SMILES: O=C(Nc1ccc(OC(F)(F)F)cc1)N(Cc1ccn2nccc2c1)C1CC1]

### 1-cyclopropyl-1-(pyrazolo[1,5-a]pyridin-5-ylmethyl)-3-(4-(trifluoromethoxy)phenyl)urea (9)

¹H NMR (600 MHz, CDCl₃) δ (ppm) 8.39 (d, J=7.2 Hz, 1H), 7.92 (d, J=2.4 Hz, 1H), 7.50 (dd, J=7.2 Hz, 2H), 7.41 (d, J=9.6 Hz, 2H), 7.16 (d, J=8.4 Hz, 2H), 6.75 (dd, J=7.2 Hz, 1H), 6.45 (d, J=1.8 Hz, 1H), 4.62 (s, 2H), 2.59 (m, 1H), 0.99 m, 2H), 0.93 (m, 2H). HRMS (ESI) m/z calcd for C₁₉H₁₇F₃N₄O₂ [M+H]⁺ 391.1304, found 391.1375.

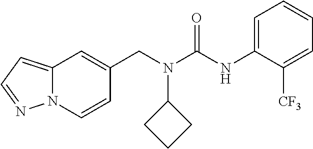 [SMILES: O=C(Nc1ccccc1C(F)(F)F)N(Cc1ccn2nccc2c1)C1CCC1]

### 1-cyclobutyl-1-(pyrazolo[1,5-a]pyridin-5-ylmethyl)-3-(2-(trifluoromethyl)phenyl)urea (10)

¹H NMR (600 MHz, CDCl₃) δ (ppm) 8.43 (d, J=7.2 Hz, 1H), 8.13 (d, J=8.4 Hz, 1H), 7.94 (d, J=2.4 Hz, 1H), 7.51 (m, 2H), 7.39 (s, 1H), 7.13 (m, 1H), 6.77 (s, 1H), 6.68 (dd, J=7.2 Hz, 1H), 6.46 (d, J=1.8 Hz, 1H), 4.66 (s, 2H), 4.45 (m 1H), 2.30 (m, 2H), 2.16 (m, 2H), 1.74 (m, 2H).

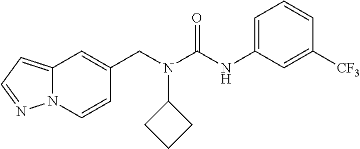 [SMILES: O=C(Nc1cccc(C(F)(F)F)c1)N(Cc1ccn2nccc2c1)C1CCC1]

### 1-cyclobutyl-1-(pyrazolo[1,5-a]pyridin-5-ylmethyl)-3-(3-(trifluoromethyl)phenyl)urea (11)

¹H NMR (600 MHz, CDCl₃) δ (ppm) 8.41 (d, J=7.2 Hz, 1H), 7.93 (d, J=2.4 Hz, 1H), 7.67 (s, 1H), 7.52 (d, J=8.4 Hz, 1H), 7.39 (s, 1H), 7.36 (m, 1H), 7.27 (m, 1H), 6.66 (dd, J=7.2 Hz, 1H), 6.48 (d, J=19.2 Hz, 2H), 4.63 (s, 2H), 4.42 (m, 1H), 2.30 (m, 2H), 2.19 (m, 2H), 1.74 (m, 2H).

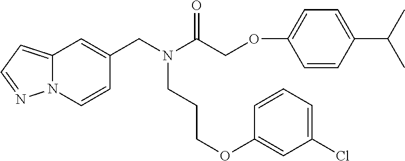 [SMILES: CC(C)c1ccc(OCC(=O)N(CCCOc2cccc(Cl)c2)Cc2ccn3nccc3c2)cc1]

### N-(3-(3-chlorophenoxy)propyl)-2-(4-isopropylphenoxy)-N-(pyrazolo[1,5-a]pyridin-5-ylmethyl)acetamide (12)

¹H NMR (600 MHz, CDCl₃) δ (ppm) 8.49 (dd, J=13.8 Hz, 1H), 7.95 (dd, J=9.0 Hz, 1H), 7.34, 7.28 (ds, 1H), 7.16 (m, 2H), 7.09 (d, J=8.4 Hz, 1H), 6.93 (m, 1H), 6.87 (d, J=8.4 Hz, 1H), 6.78 (d, J=10.8 Hz, 1H), 6.77 (d, J=9.0 Hz, 1H), 6.68 (dd, J=8.4 Hz, 1H), 6.61 (dd, J=20.4 Hz, 1H), 6.45 (dd, J=9.0 Hz, 1H), 4.78 (s, 1H), 4.71 (s, 1H), 4.66 (d, J=9.0 Hz, 2H), 3.95, 3.87 (dt, 2H), 3.61, 3.55 (dt, 2H), 2.84 (m, 1H), 2.06 (m, 1H), 1.20 (dd, J=22.8 Hz, 6H).

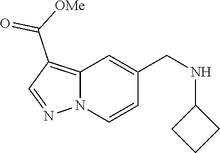 [SMILES: COC(=O)c1cnn2ccc(CNC3CCC3)cc12]

### methyl 5-((cyclobutylamino)methyl)pyrazolo[1,5-a]pyridine-3-carboxylate (13)

¹H NMR (600 MHz, CDCl₃) δ (ppm) 8.45 (d, J=7.2 Hz, 1H), 8.36 (s, 1H), 8.06 (s, 1H), 7.07 (dd, J=7.2 Hz, 1H), 3.90 (s, 3H), 3.85 (s, 2H), 3.32 (m, 1H), 2.23 (m, 2H), 1.83 (m, 2H), 1.76, 1.74 (m, 2H).

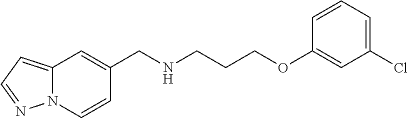 [SMILES: Clc1cccc(OCCCNCc2ccn3nccc3c2)c1]

### 3-(3-chlorophenoxy)-N-(pyrazolo[1,5-a]pyridin-5-ylmethyl)propan-1-amine (14)

¹H NMR (600 MHz, CDCl₃) δ (ppm) 8.36 (s, 1H), 7.89 (d, J=2.4 Hz, 1H), 7.42 (s, 1H), 7.15 (t, J=8.4 Hz, 1H), 6.88 (dd, J=18.0 Hz, 1H), 6.87 (s, 1H), 6.73 (dd, J=17.4 Hz, 1H), 6.40 (s, 1H), 4.02 (t, J=6.0 Hz, 2H), 3.80 (s, 2H), 2.81 (t, J=6.6 Hz, 2H), 1.96 (m, 2H), 1.81 (bs, 1H).

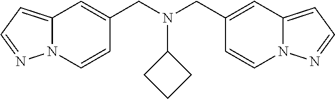 [SMILES: c1cc2cc(CN(Cc3ccn4nccc4c3)C3CCC3)ccn2n1]

### N,N-bis(pyrazolo[1,5-a]pyridin-5-ylmethyl)cyclobutanamine (15)

The compound is an over-reductive amination product. ¹H NMR (600 MHz, CDCl₃) δ (ppm) 8.38 (d, J=7.2 Hz, 2H), 7.89 (s, 2H), 7.38 (s, 2H), 6.77 (d, J=4.8 Hz, 2H), 6.41 (s, 2H), 3.50 (s, 4H), 3.22 (m, 1H), 1.97 (m, 2H), 1.88 (m, 2H), 1.68, 1.63 (m, 2H).

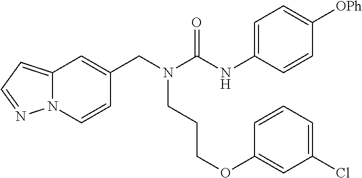 [SMILES: O=C(Nc1ccc(Oc2ccccc2)cc1)N(CCCOc1cccc(Cl)c1)Cc1ccn2nccc2c1]

### 1-(3-(3-chlorophenoxy)propyl)-3-(4-phenoxyphenyl)-1-(pyrazolo[1,5-a]pyridin-5-ylmethyl)urea (16)

¹H NMR (600 MHz, CDCl₃) δ (ppm) 8.55 (d, J=7.2 Hz, 1H), 7.96 (d, J=2.4 Hz, 1H), 7.47 (s, 1H), 7.30 (t, J=7.8 Hz, 2H), 7.20 (m, 3H), 6.90 (m, 6H), 6.80 (m, 2H), 6.51 (d, J=1.8 Hz, 1H), 4.63 (s, 2H), 4.08 (t, J=5.4 Hz, 2H), 3.58 (t, J=6.6 Hz, 2H), 2.11 (m, 2H).

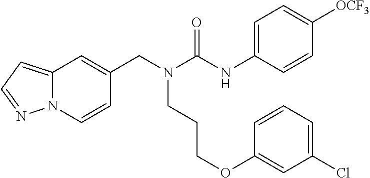 [SMILES: O=C(Nc1ccc(OC(F)(F)F)cc1)N(CCCOc1cccc(Cl)c1)Cc1ccn2nccc2c1]

### 1-(3-(3-chlorophenoxy)propyl)-1-(pyrazolo[1,5-a]pyridin-5-ylmethyl)-3-(4-trifluoromethoxy)phenyl)urea (17)

¹H NMR (600 MHz, CDCl₃) δ (ppm) 8.53 (d, J 7.2 Hz, 1H), 7.96 (d, J 2.4 Hz, 1H), 7.46 (s, 1H), 7.25 (dd, J=7.2 Hz, 2H), 7.21 (t, J=8.4 Hz, 1H), 7.06 (m, 3H), 6.99 (dd, J=8.4 Hz, 1H), 6.93 (t, J=2.4 Hz, 1H), 6.80 (m, 2H), 6.50 (d, J=1.8 Hz, 1H), 4.63 (s, 2H), 4.08 (t, J=5.4 Hz, 2H), 3.58 (t, J=6.6 Hz, 2H), 2.09 (t, J=6.0 Hz, 2H).

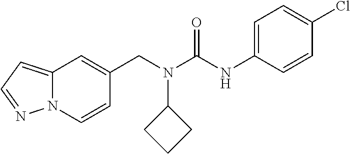 [SMILES: O=C(Nc1ccc(Cl)cc1)N(Cc1ccn2nccc2c1)C1CCC1]

### 3-(4-chlorophenyl)-1-cyclobutyl-1-(pyrazolo[1,5-a]pyridin-5-ylmethyl)urea (18)

¹H NMR (600 MHz, CDCl₃) δ (ppm) 8.51 (d, J=7.2 Hz, 1H), 7.95 (d, J=2.4 Hz, 1H), 7.42 (s, 1H), 7.29 (d, J=9.0 Hz, 2H), 7.21 (d, J=9.0 Hz, 2H), 6.72 (dd, J=7.2 Hz, 1H), 6.48 (d, J=1.8 Hz, 1H), 6.37 (s, 1H), 4.63 (s, 2H), 4.41 (m, 1H), 2.16 (m, 2H), 1.72 (m, 2H).

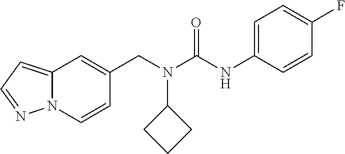 [SMILES: O=C(Nc1ccc(F)cc1)N(Cc1ccn2nccc2c1)C1CCC1]

### 1-cyclobutyl-3-(4-fluorophenyl)-1-(pyrazolo[1,5-a]pyridin-5-ylmethyl)urea (19)

¹H NMR (600 MHz, CDCl₃) δ (ppm) 8.52 (d, J=7.2 Hz, 7.95 (d, J=2.4 Hz, 1H), 7.43 s, 1H), 7.29 (dd, J=9.0 Hz, 2H), 6.96 (t, J=9.0 Hz, 2H), 6.73 (dd, J=7.2 Hz, 1H), 6.48 (d, J=2.4 Hz, 1H), 6.32 (s, 1H), 4.63 (s, 2H), 4.42 (m, 1H), 2.30 (m, 2H), 2.16 (m, 2H), 1.72 (m, 2H).

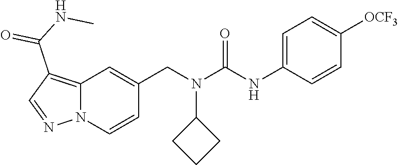 [SMILES: CNC(=O)c1cnn2ccc(CN(C(=O)Nc3ccc(OC(F)(F)F)cc3)C3CCC3)cc12]

### 5-((1-cyclobutyl-3-(4-(trifluoromethoxy)phenyl)ureido)methyl)-N-methylpyrazolo[1,5-a]pyridine-3-carboxamide (20)

¹H NMR (600 MHz, CDCl₃) δ (ppm) 8.60 (d, J=7.2 Hz, 1H), 8.38 (s, 1H), 8.17 (s, 1H), 7.53 (d, J=9.0 Hz, 2H), 7.21 (d, J=9.0 Hz, 2H), 7.03 (dd, J=7.2 Hz, 1H), 4.85 (s, 2H), 2.95 (s, 3H), 2.34 (m, 2H), 2.16 (m, 2H), 1.72 (m, 2H).

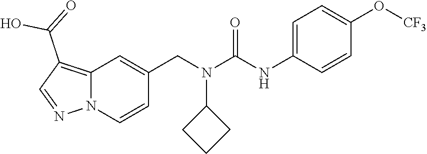 [SMILES: O=C(O)c1cnn2ccc(CN(C(=O)Nc3ccc(OC(F)(F)F)cc3)C3CCC3)cc12]

### 5-((1-cyclobutyl-3-(4-(trifluoromethoxy)phenyl)ureido)methyl)pyrazolo[1,5-a]pyridine-3-carboxylic acid (21)

¹H NMR (600 MHz, CDCl₃) δ (ppm) 8.65 (d, J=7.2 Hz, 1H), 8.39 (s, 1H), 8.08 (s, 1H), 7.52 (dd, J=7.2 Hz, 2H), 7.21 (d, J=8.4 Hz, 2H), 7.08 (dd, J=8.4 Hz, 1H), 4.87 (s, 2H), 4.68 (m, 1H), 2.32 (m, 2H), 2.23 (m, 2H), 1.77 (m, 2H). HRMS (ESI) m % z calcd for C₂H₁₉F₃N₄O₄ [M+H]⁺ 449.1358, found 449.1426.

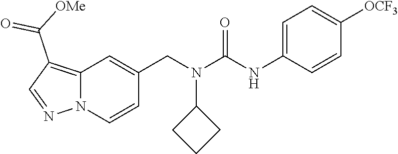 [SMILES: COC(=O)c1cnn2ccc(CN(C(=O)Nc3ccc(OC(F)(F)F)cc3)C3CCC3)cc12]

### methyl 5-((1-cyclobutyl-3-(4-(trifluoromethoxy)phenyl)ureido)methyl)pyrazolo[1,5-a]pyridine-3-carboxylate (22)

¹H NMR (600 MHz, CDCl₃) δ (ppm) 8.46 (d, J=7.2 Hz, 1H), 8.36 (s, 1H), 8.02 (s, 1H), 7.39 (d, J=9.0 Hz, 2H), 7.13 (d, J=7.2 Hz, 2H), 6.93 (dd, J=7.2 Hz, 1H), 6.44 (s, 1H), 4.72 (s, 2H), 4.33 (m, 1H), 3.89 (s, 3H), 2.33 (m, 2H), 2.22 (m, 2H), 1.79 (m, 2H).

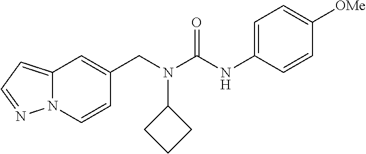 [SMILES: COc1ccc(NC(=O)N(Cc2ccn3nccc3c2)C2CCC2)cc1]

### 1-cyclobutyl-3-(4-methoxyphenyl)-1-(pyrazolo[1,5-a]pyridin-5-ylmethyl)urea (23)

¹H NMR (600 MHz, CDCl₃) δ (ppm) 8.49 (d, J=7.2 Hz, 1H), 7.94 (d, J=2.4 Hz, 1H), 7.43 (s, 1H), 7.25 (d, J=8.4 Hz, 2H), 6.82 (d, J=8.4 Hz, 2H), 6.72 (dd, J=7.2 Hz, 1H), 6.48 (d, J=1.8 Hz, 1H), 6.23 (s, 1H), 4.63 (s, 2H), 4.42 (m, 1H), 3.77 (s, 3H), 2.29 (m, 2H), 2.17 (m, 2H), 1.75 (m, 2H).

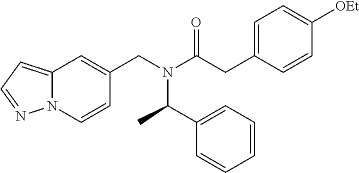 [SMILES: CCOc1ccc(CC(=O)N(Cc2ccn3nccc3c2)[C@H](C)c2ccccc2)cc1]

### (R)-2-(4-ethoxyphenyl)-N-(1-phenylethyl)-N-(pyrazolo[1,5-a]pyridin-5-ylmethyl)acetamide (24)

¹H NMR (600 MHz, CDCl₃) δ (ppm) 8.47, 8.41 (d, J=7.2 Hz, 1H), 7.95, 7.91 (s, 1H), 7.25 (m, 5H), 7.06 (m, 2H), 6.90 (d, J=8.4 Hz, 1H), 6.81 (d, J=8.4 Hz, 1H), 6.56, 6.40 (d, J=7.2 Hz, 1H), 6.44, 6.38 (s, 1H), 6.22, 5.35 (m, 1H), 4.61, 4.38 (d, J=15.6, 1), 4.15 (m, 1H), 4.05 (m, 2H), 3.98 (m, 1H), 3.58 (m, 1H), 1.48, 1.42 (m, 6H).

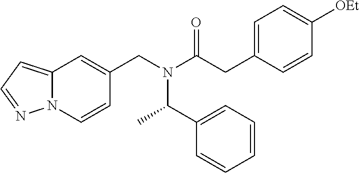 [SMILES: CCOc1ccc(CC(=O)N(Cc2ccn3nccc3c2)[C@@H](C)c2ccccc2)cc1]

### (S)-2-(4-ethoxyphenyl)-N-(1-phenylethyl)-N-(pyrazolo[1,5-a]pyridin-5-ylmethyl)acetamide (25)

¹H NMR (600 MHz, CDCl₃) δ (ppm) 8.33, 8.26 (d, J=7.2 Hz, 1H), 7.90, 7.84 (s, 1H), 7.25 (m, 5H), 7.04 (m, 2H), 6.87 (d, J=8.4 Hz, 1H), 6.78 (d, J=8.4 Hz, 1H), 6.49, 6.36 (d, J=7.2 Hz, 1H), 6.40, 6.30 (s, 1H), 6.19, 5.32 (m, 1H), 4.64, 4.35 (d, J=15.6 Hz, 1H), 4.13, 3.96 (d, J=18.0 Hz, 1H), 4.00 (m, 2H), 3.86 (m, 1H), 3.56 (m, 1H), 1.44, 1.38 (m, 6H). HRMS (ESI) m/z calcd for C₂₆H₂₇N₃O₂ [M+H]⁺ 414.2103, found 414.2174.

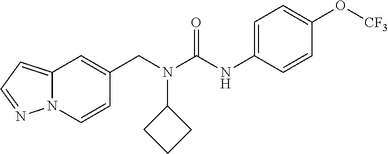 [SMILES: O=C(Nc1ccc(OC(F)(F)F)cc1)N(Cc1ccn2nccc2c1)C1CCC1]

### 1-cyclobutyl-1-(pyrazolo[1,5-a]pyridin-5-ylmethyl)-3-(4-(trifluoromethoxy)phenyl)urea (26)

¹H NMR (600 MHz, CDCl₃) δ (ppm) 8.41 (d, J=7.2 Hz, 1H), 7.93 (d, J=2.4 Hz, 1H), 7.39 (s, 1H), 7.36 (d, J=9.0 Hz, 2H), 7.11 (d, J=9.0 Hz, 2H), 6.64 (d, J=7.2 Hz, 1H), 6.46 (d, J=1.8 Hz, 2H), 4.61 (s, 2H), 4.43 (m, 1H), 2.29 (m, 2H), 2.15 (m, 2H), 1.72 (m, 2H). HRMS (ESI) m/z calcd for C₂₀H₁₉F₃N₄O₂ [M+H]⁺ 405.1460, found 405.1523.

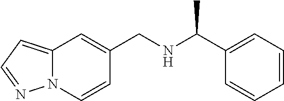 [SMILES: C[C@H](NCc1ccn2nccc2c1)c1ccccc1]

### (S)-1-phenyl-N-(pyrazolo[1,5-a]pyridin-5-ylmethyl)ethan-1-amine (27)

¹H NMR (600 MHz, CDCl₃) δ (ppm) 8.39 (d, J=7.2 Hz, 1H), 7.91 (d, J=2.4 Hz, 1H), 7.42 (s, 1H), 7.36 (d, J=4.2 Hz, 4H), 7.28 (m, 1H), 6.72 (d, J=6.0 Hz, 1H), 6.43 (d, J=1.8 Hz, 1H), 3.84 (m, 1H), 3.65 (m, 2H), 1.68 (bs, 1H), 1.41 (d, J=6.6 Hz, 3H).

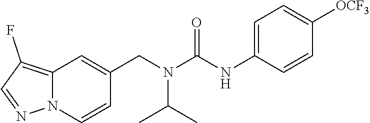 [SMILES: CC(C)N(Cc1ccn2ncc(F)c2c1)C(=O)Nc1ccc(OC(F)(F)F)cc1]

### 1-((3-fluoropyrazolo[1,5-a]pyridin-5-yl)methyl)-1-isopropyl-3-(4-(trifluoromethoxy)phenyl) urea (28)

¹H NMR (600 MHz, CDCl₃) δ (ppm) 8.29 (d, J=6.8 Hz, 1H), 7.82 (d, J=3.4 Hz, 1H), 7.46 (s, 1H), 7.31 (d, J=8.8 Hz, 2H), 7.11 (d, J=8.4 Hz, 2H), 6.71 (d, J=6.5 Hz, 1H), 6.34 (s, 1H), 4.53 (dd, J=13.0, 6.5 Hz, 1H), 4.48 (s, 2H), 1.27 (d, J=6.7 Hz, 6H).

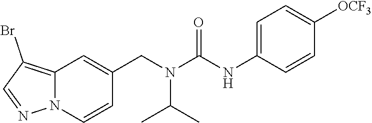 [SMILES: CC(C)N(Cc1ccn2ncc(Br)c2c1)C(=O)Nc1ccc(OC(F)(F)F)cc1]

### 1-((3-bromopyrazolo[1,5-a]pyridin-5-yl)methyl)-1-isopropyl-3-(4-(trifluoromethoxy) phenyl) urea (29)

¹H NMR (600 MHz, CDCl₃) δ (ppm) 8.43 (d, J=7.2 Hz, 1H), 7.94 (s, 1H), 7.44 (s, 1H), 7.31 (d, J=9.0 Hz, 2H), 7.12 (d, J=8.5 Hz, 2H), 6.81 (d, J=6.2 Hz, 1H), 6.34 (s, 1H), 4.53 (s, 3H), 1.27 (d, J=6.7 Hz, 6H).

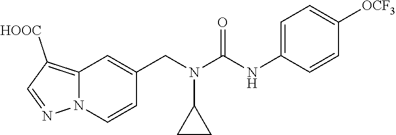 [SMILES: O=C(O)c1cnn2ccc(CN(C(=O)Nc3ccc(OC(F)(F)F)cc3)C3CC3)cc12]

### 5-((1-cyclopropyl-3-(4-(trifluoromethoxy)phenyl)ureido)methyl)pyrazolo[1,5-a]pyridine-3-carboxylic acid (30)

¹H NMR (600 MHz, CDCl₃) δ 8.44 (s. 1H), 8.35 (d, J=8.4 Hz, 1H), 7.99 (d, J=7.8 Hz, 1H), 7.46 (t, J=8.4 Hz, 2H), 7.14 (t, J=7.8 Hz, 2H), 6.95 (s, 1H), 4.67 (d, J=7.8 Hz, 2H), 2.61 (m, 1H), 0.99 (t, J=6.6 Hz, 2H), 0.91 (d, J=3.6 Hz, 2H).

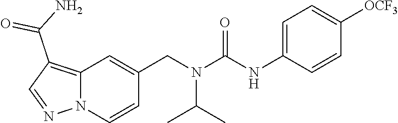 [SMILES: CC(C)N(Cc1ccn2ncc(C(N)=O)c2c1)C(=O)Nc1ccc(OC(F)(F)F)cc1]

### 5-((1-isopropyl-3-(4-(trifluoromethoxy)phenyl)ureido)methyl)pyrazolo[1,5-a]pyridine-3-carboxamide (31)

¹H NMR (600 MHz, CDCl₃) δ 8.37 (d, J=7.2 Hz, 1H), 8.15 (s, 1H), 8.12 (s, 1H), 7.29 (d, J=9.0 Hz, 2H), 7.02 (d, J=8.4 Hz, 2H), 6.59 (s, 1H), 4.51 (d, J=7.8 Hz, 2H), 4.39 (m, 1H), 1.19 (d, J=6.6 Hz, 6H).

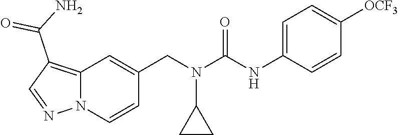 [SMILES: NC(=O)c1cnn2ccc(CN(C(=O)Nc3ccc(OC(F)(F)F)cc3)C3CC3)cc12]

### 5-((1-cyclopropyl-3-(4-(trifluoromethoxy)phenyl)ureido)methyl)pyrazolo[1,5-a]pyridine-3-carboxamide (32)

¹H NMR (600 MHz, CDCl₃) δ 8.33 (d, J=7.2 Hz, 1H), 8.07 (d, J=12.6 Hz, 1H), 7.42 (d, J=9.0 Hz, 2H), 7.08 (d, J=8.4 Hz, 2H), 6.90-6.89 (m, 1H), 5.80 (bs, 2H), 4.62 (s, 2H), 2.62-2.60 (m, 1H), 0.94 (d, J=5.4 Hz, 2H), 0.86 (d, J=3.0 Hz, 2H).

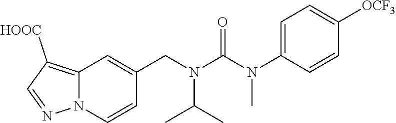 [SMILES: CC(C)N(Cc1ccn2ncc(C(=O)O)c2c1)C(=O)N(C)c1ccc(OC(F)(F)F)cc1]

### 5-((1-isopropyl-3-methyl-3-(4-(trifluoromethoxy)phenyl)ureido)methyl)pyrazolo[1,5-a]pyridine-3-carboxylic acid (33)

¹H NMR (600 MHz, CDCl₃) δ 8.52 (d, J=7.2 Hz, 1H), 8.47 (s, 1H), 8.06 (s, 1H), 7.29 (d, J=8.4 Hz, 2H), 7.19 (d, J=8.4 Hz, 2H), 6.96-6.95 (m, 1H), 4.28 (s, 2H), 4.12-4.10 (m, 1H), 1.04 (d, J=6.6 Hz, 6H).

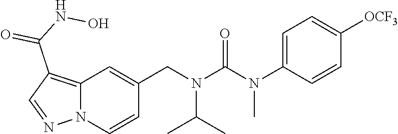 [SMILES: CC(C)N(Cc1ccn2ncc(C(=O)NO)c2c1)C(=O)N(C)c1ccc(OC(F)(F)F)cc1]

### N-hydroxy-5-((1-isopropyl-3-methyl-3-(4-(trifluoromethoxy) phenyl)ureido)methyl) pyrazolo[1,5-a]pyridine-3-carboxamide (34)

¹H NMR (600 MHz, CDCl₃) δ 8.41 (t, J=7.2 Hz, 1H), 8.27 (s, 1H), 8.02 (d, J=6.6 Hz, 1H), 7.29 (d, J=9.0 Hz, 2H), 7.23 (d, J=8.4 Hz, 2H), 7.09 (m, 1H), 6.79 (s, 1H), 4.17 (d, J=7.8 Hz, 2H), 4.05-4.03 (m, 1H), 3.09 (s, 3H), 0.99 (t, J=7.2 Hz, 6H).

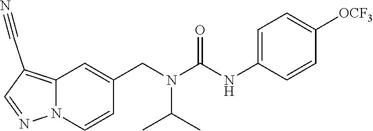 [SMILES: CC(C)N(Cc1ccn2ncc(C#N)c2c1)C(=O)Nc1ccc(OC(F)(F)F)cc1]

### 1-((3-cyanopyrazolo[1,5-a]pyridin-5-yl)methyl)-1-isopropyl-3-(4-(trifluoromethoxy)phenyl)urea (35)

¹H NMR (600 MHz, CDCl₃) δ 8.52 (d, J=7.2 Hz, 1H), 8.24 (s, 1H), 7.68 (s, 1H), 7.41 (d, J=9.0 Hz, 2H), 7.17 (d, J=8.4 Hz, 2H), 7.08-7.06 (m, 1H), 6.51 (s, 1H), 4.62 (s, 2H), 4.34-4.32 (m, 1H), 1.32 (d, J=6.6 Hz, 6H).

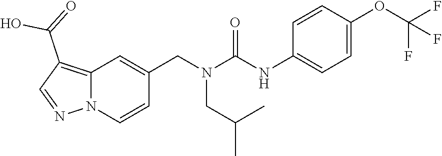 [SMILES: CC(C)CN(Cc1ccn2ncc(C(=O)O)c2c1)C(=O)Nc1ccc(OC(F)(F)F)cc1]

### 5-((1-isobutyl-3-(4-(trifluoromethoxy)phenyl)ureido)methyl)pyrazolo[1,5-a]pyridine-3-carboxylic acid (36)

¹H NMR (600 MHz, CDCl₃) δ 8.54 (d, J=7.2 Hz, 1H), 8.38 (s. 1H), 7.98 (s, 1H), 7.33 (d, J=9.0 Hz, 2H), 7.08 (d, J=8.4 Hz, 2H), 6.96 (m, 1H), 6.40 (s, 1H), 4.66 (s, 2H), 3.11 (d, J=7.8 Hz, 2H), 2.02 (m, 1H), 0.96 (d, J=6.6 Hz, 6H).

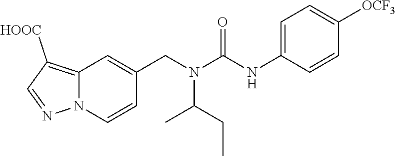 [SMILES: CCC(C)N(Cc1ccn2ncc(C(=O)O)c2c1)C(=O)Nc1ccc(OC(F)(F)F)cc1]

### 5-((1-(sec-butyl)-3-(4-(trifluoromethoxy)phenyl)ureido)methyl)pyrazolo[1,5-a]pyridine-3-carboxylic acid (37)

¹H NMR (600 MHz, CDCl₃) δ 8.52 (d, J=7.2 Hz, 1H), 8.47 (s, 1H), 8.15 (s, 1H), 7.36 (d, J=9.0 Hz, 2H), 7.13 (d, J=8.4 Hz, 2H), 7.04 (m, 1H), 6.57 (s, 1H), 4.65 (d, J=17.4 Hz, 1H), 4.54 (d, J=17.4 Hz, 1H), 4.21 (m, 1H), 1.70 (m, 1H), 1.57 (m, 1H), 1.26 (d, J=6.6 Hz, 3H), 0.98 (t, J=7.2 Hz, 3H).

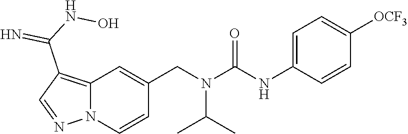 [SMILES: CC(C)N(Cc1ccn2ncc(C(=N)NO)c2c1)C(=O)Nc1ccc(OC(F)(F)F)cc1]

### N-hydroxy-5-((1-isopropyl-3-(4-(trifluoromethoxy)phenyl)ureido)methyl)pyrazolo[1,5-a]pyridine-3-carboximidamide (38)

¹H NMR (600 MHz, CDCl₃) δ 8.42 (t, J=7.8 Hz, 1H), 8.23, 8.16 (s, 1H), 8.11, 7.92 (s, 1H), 7.33 (t, J=9.0 Hz, 2H), 7.01 (d, J=9.0 Hz, 2H), 6.82 (d, J=7.2 Hz, 1H), 4.53 (s, 2H), 4.46-4.44 (m, 1H), 1.26-1.24 (m, 6H).

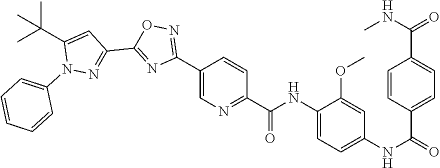 [SMILES: CNC(=O)c1ccc(C(=O)Nc2ccc(NC(=O)c3ccc(-c4noc(-c5cc(C(C)(C)C)n(-c6ccccc6)n5)n4)cn3)c(OC)c2)cc1]

### N¹-(4-(5-(5-(5-(tert-butyl)-1-phenyl-1H-pyrazol-3-yl)-1,2,4-oxadiazol-3-yl)picolinamido)-3-methoxyphenyl)-N⁴-methylterephthalamide (39)

¹H NMR (600 MHz, DMSO-d₆) δ (ppm) 10.40 (d, J=3.6 Hz, 2H), 9.36 (s, 1H), 8.72 (d, J=1.8 Hz, 1H), 8.71 (d, J=2.4 Hz, 1H), 8.60 (b, J=4.8 Hz, 1H), 8.39 (t, J=8.4 Hz, 2H), 8.04 (d, J=8.4 Hz, 2H), 7.97 (d, J=8.4 Hz, 2H), 7.71 (m, 2H), 7.57 (d, J=8.4 Hz, 2H), 7.46 (dd, J=8.4 Hz, 1H), 7.10 (s, 1H), 3.96 (s, 3H), 2.82 (d, J=4.8 Hz, 3H), 1.22 (s, 9H).

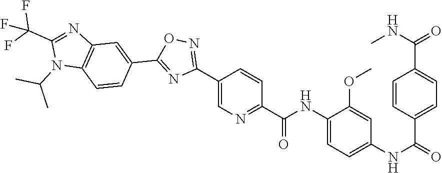 [SMILES: CNC(=O)c1ccc(C(=O)Nc2ccc(NC(=O)c3ccc(-c4noc(-c5ccc6c(c5)nc(C(F)(F)F)n6C(C)C)n4)cn3)c(OC)c2)cc1]

### N¹-(4-(5-(5-(1-isopropyl-2-(trifluoromethyl)-1H-benzo[d]imidazol-5-yl)-1,2,4-oxadiazol-3-yl)picolinamido)-3-methoxyphenyl)-N⁴-methylterephthalamide (40)

¹H NMR (600 MHz, CDCl₃) δ (ppm) 10.40 (d, J=3.6 Hz, 2H), 9.36 (s, 1H), 8.66 (d, J=3.6 Hz, 1H), 8.40 (d, J=8.4 Hz, 1H), 8.24 (m, 1H), 7.95 (d, J=4.2 Hz, 2H), 7.89 (d, J=8.4 Hz, 1H), 7.83 (d, J=8.4 Hz, 1H), 7.38 (dd, J=8.4 Hz, 1H), 4.98 (m, 1H), 4.05 (s, 3H), 2.98 (s, 3H), 1.74 (d, J=7.2 Hz, 6H).

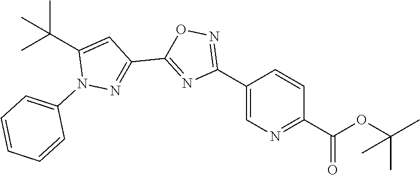 [SMILES: CC(C)(C)OC(=O)c1ccc(-c2noc(-c3cc(C(C)(C)C)n(-c4ccccc4)n3)n2)cn1]

### tert-butyl 5-(5-(5-(tert-butyl)-1-phenyl-1H-pyrazol-3-yl)-1,2,4-oxadiazol-3-yl)picolinate (41)

¹H NMR (600 MHz, CDCl₃) δ (ppm) 9.51 (d, J=1.2 Hz, 1H), 8.57 (dd, J=8.4 Hz, 1H), 8.14 (d, J=8.4 Hz, 1H), 7.51 (m, 3H), 7.44 (d, J=7.8 Hz, 2H), 6.96 (s, 1H), 1.66 (s, 9H), 1.26 (s, 9H).

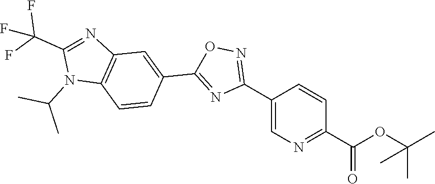 [SMILES: CC(C)n1c(C(F)(F)F)nc2cc(-c3nc(-c4ccc(C(=O)OC(C)(C)C)nc4)no3)ccc21]

### tert-butyl 5-(5-(1-isopropyl-2-(trifluoromethyl)-1H-benzo[d]imidazol-5-yl)-1,2,4-oxadiazol-3-yl)picolinate (42)

¹H NMR (600 MHz, CDCl₃) δ (ppm) 9.52 (d, J=1.2 Hz, 1H), 8.76 (d, J=1.2 Hz, 1H), 8.57 (dd, J=8.4 Hz, 1H), 8.26 (dd, J=8.4 Hz, 1H), 8.19 (dd, J=8.4 Hz, 1H), 7.82 (d, J=8.4 Hz, 1H), 4.99 (m, 1H), 1.76 (d, J=7.2 Hz, 6H), 1.67 (s, 9H).

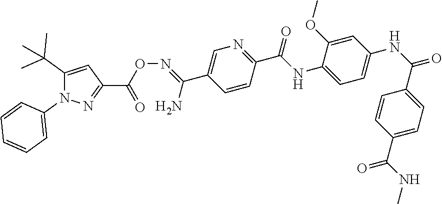 [SMILES: CNC(=O)c1ccc(C(=O)Nc2ccc(NC(=O)c3ccc(/C(N)=N/OC(=O)c4cc(C(C)(C)C)n(-c5ccccc5)n4)cn3)c(OC)c2)cc1]

### (Z)-N¹-(4-(5-(N′-((5-(tert-butyl)-1-phenyl-1H-pyrazole-3-carbonyl)oxy)carbamimidoyl)picolinamido)-3-methoxyphenyl)-N⁴-methylterephthalamide (43)

¹H NMR (600 MHz, CDCl₃) δ (ppm) 8.86 (s, 1H), 8.38 (d, J=8.4 Hz, 1H), 8.31 (d, J=8.4 Hz, 1H), 8.14 (d, J=8.4 Hz, 1H), 7.87 (d, J=7.8 Hz, 2H), 7.78 (d, J=7.8 Hz, 2H), 7.70 (s, 1H), 7.38 (m, 10H), 7.04 (d, J=9.0 Hz, 1H), 3.93 (s, 3H), 2.89 (s, 3H), 1.09 (s, 9H).

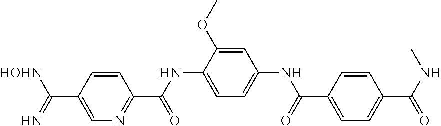

### N1-(4-(5-(N-hydroxycarbamimidoyl)picolinamido)-3-methoxyphenyl)-N4-methylterephthalamide (Int-1)

¹H NMR (600 MHz, DMSO-d₆) δ (ppm) 10.42 (s, 1H), 10.32 (s, 1H), 9.21 (s, 1H), 8.61 (d, J=4.8 Hz, 2H), 8.33 (m, 2H), 8.04 (d, J=7.8 Hz, 2H), 7.97 (d, J=7.8 Hz, 2H), 7.71 (s, 1H), 7.47 (d, J=7.8 Hz, 1H), 6.43 (bs, 1H), 6.12 (s, 1H), 5.69 (bs, 1H), 5.16 (bs, 1H), 3.95 (s, 3H), 2.82 (s, 3H).

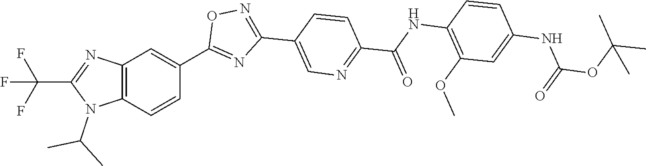 [SMILES: COc1cc(NC(=O)OC(C)(C)C)ccc1NC(=O)c1ccc(-c2noc(-c3ccc4c(c3)nc(C(F)(F)F)n4C(C)C)n2)cn1]

### tert-butyl (4-(5-(5-(1-isopropyl-2-(trifluoromethyl)-1H-benzo[d]imidazol-5-yl)-1,2,4-oxadiazol-3-yl)picolinamido)-3-methoxyphenyl)carbamate (44)

¹H NMR (600 MHz, CDCl₃) δ (ppm) 10.39 (d, J=9.6 Hz, 1H), 9.05 (s, 1H), 8.44 (d, J=7.8 Hz, 1H), 8.33 (m, 2H), 7.42 (s, 1H), 6.66 (s, 1H), 6.53 (s, 1H), 6.28 (bs, 1H), 5.81 (bs, 1H), 4.94 (m, 1H), 3.97 (s, 3H), 1.72 (d, J=7.2 Hz, 3H), 1.53 (d, J=7.2 Hz, 3H), 1.52 (s, 9H).

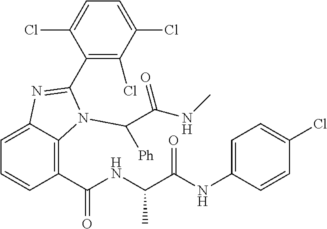 [SMILES: CNC(=O)C(c1ccccc1)n1c(-c2c(Cl)ccc(Cl)c2Cl)nc2cccc(C(=O)N[C@@H](C)C(=O)Nc3ccc(Cl)cc3)c21]

### N-((S)-1-((4-chlorophenyl)amino)-1-oxopropan-2-yl)-1-(2-(methylamino)-2-oxo-1-phenylethyl)-2-(2,3,6-trichlorophenyl)-1H-benzo[d]imidazole-7-carboxamide (45)

¹H NMR (600 MHz, CDCl₃) δ (ppm) 9.31 (bs, 1H), 7.91 (d, J=7.8 Hz, 1H), 7.45 (bs, 2H), 7.37 (m, 1H), 7.18 (m, 1H), 7.01 (m, 3H), 6.91 (m, 2H), 6.75 (m, 2H), 6.48 (s, 1H), 5.89 (bs, 1H), 4.82 (m, 1H), 3.26 (m, 1H), 3.04 (m, 1H), 2.66 (s, 3H), 1.25 (m, 3H).

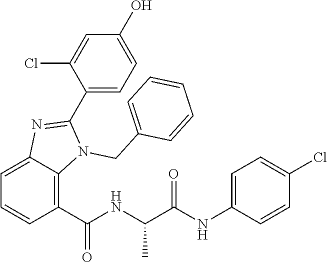 [SMILES: C[C@H](NC(=O)c1cccc2nc(-c3ccc(O)cc3Cl)n(Cc3ccccc3)c12)C(=O)Nc1ccc(Cl)cc1]

### (S)-1-benzyl-2-(2-chloro-4-hydroxyphenyl)-N-(1-((4-chlorophenyl)amino)-1-oxopropan-2-yl)-1H-benzo[d]imidazole-7-carboxamide (46)

¹H NMR (600 MHz, CDCl₃) δ (ppm) 7.94 (d, J=8.4 Hz, 1H), 7.49 (d, J=8.4 Hz, 2H), 7.37 (d, J=7.8 Hz, 1H), 7.32 (d, J=7.8 Hz, 1H), 7.27 (m, 3H), 7.05 (m, 3H), 6.96 (s, 1H), 6.76 (d, J=8.4 Hz, 1H), 6.67 (d, J=7.2 Hz, 2H), 5.51 (bs, 2H), 4.35 (m, 1H), 1.25 (m, 3H).

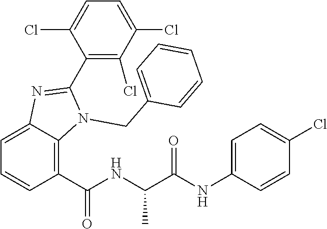 [SMILES: C[C@H](NC(=O)c1cccc2nc(-c3c(Cl)ccc(Cl)c3Cl)n(Cc3ccccc3)c12)C(=O)Nc1ccc(Cl)cc1]

### (S)-1-benzyl-N-(1-((4-chlorophenyl)amino)-1-oxopropan-2-yl)-2-(2,3,6-trichlorophenyl)-1H-benzo[d]imidazole-7-carboxamide (47)

¹H NMR (600 MHz, CDCl₃) δ (ppm) 8.59 (bs, 1H), 8.02 (d, J=7.8 Hz, 1H), 7.63 (m, 3H), 7.43 (m, 2H), 7.24 (d, J=7.8 Hz, 2H), 7.13 (bs, 3H), 6.69 (s, 2H), 6.24 (bs, 1H), 4.46 (m, 1H), 1.26 (m, 3H).

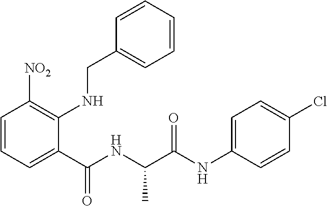 [SMILES: C[C@H](NC(=O)c1cccc([N+](=O)[O-])c1NCc1ccccc1)C(=O)Nc1ccc(Cl)cc1]

### (S)-2-(benzylamino)-N-(1-((4-chlorophenyl)amino)-1-oxopropan-2-yl)-3-nitrobenzamide (Int-2)

¹H NMR (600 MHz, CDCl₃) δ (ppm) 8.14 (d, J=8.4 Hz, 1H), 7.71 (d, J=7.8 Hz, 2H), 7.47 (d, J=7.8 Hz, 7.23 (m, 5H), 7.12 (d, J=7.2 Hz, 2H), 6.80 (t, J=7.8 Hz, 1H), 4.66 (m, 1H), 4.27 (q, J 22.8 Hz, 2H), 1.46 (d, J 7.8 Hz, 3H).

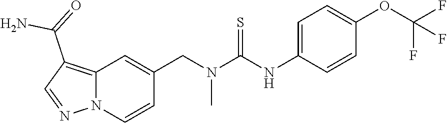 [SMILES: CN(Cc1ccn2ncc(C(N)=O)c2c1)C(=S)Nc1ccc(OC(F)(F)F)cc1]

### 5-((1-methyl-3-(4-(trifluoromethoxy)phenyl)thioureido)methyl)pyrazolo[1,5-a]pyridine-3-carboxamide (48)

¹H NMR (600 MHz, CDCl₃) δ 8.40 (d, J=7.2 Hz, 1H), 8.25 (s, 1H), 8.02 (s, 1H), 7.38 (d, J=9.0 Hz, 2H), 7.12 (d, J=8.4 Hz, 2H), 7.03 (d, J=7.2 Hz, 1H), 5.29 (s, 2H), 3.22 (s, 3H).

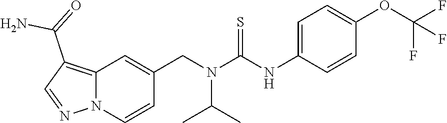 [SMILES: CC(C)N(Cc1ccn2ncc(C(N)=O)c2c1)C(=S)Nc1ccc(OC(F)(F)F)cc1]

### 5-((1-isopropyl-3-(4-(trifluoromethoxy)phenyl)thioureido)methyl)pyrazolo[1,5-a]pyridine-3-carboxamide (49)

¹H NMR (600 MHz, CDCl₃) δ 8.42 (d, J=7.2 Hz, 1H), 8.25 (s, 1H), 8.07 (s, 1H), 7.24 (d, J=7.8 Hz, 2H), 7.23 (s, 1H), 7.07 (d, J=8.4 Hz, 2H), 6.93 (d, J=7.2 Hz, 1H), 5.22 (m, 1H), 4.99 (s, 2H), 1.24 (d, J 6.6 Hz, 6H).

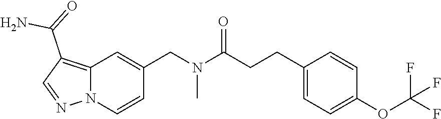 [SMILES: CN(Cc1ccn2ncc(C(N)=O)c2c1)C(=O)CCc1ccc(OC(F)(F)F)cc1]

### 5-((N-methyl-3-(4-(trifluoromethoxy)phenyl)propanamido)methyl)pyrazolo[1,5-a]pyridine-3-carboxamide (51)

¹H NMR (600 MHz, CDCl₃) δ 8.42-8.39 (m, 1H), 8.17 (s, 1H), 8.14 (s, 1H), 7.29-7.24 (m, 2H), 7.14 (d, J=8.1 Hz, 2H), 6.82 (dd, J=7.1, 2.0 Hz, 1H), 4.66 (s, 2H), 3.04 (t, J=7.6 Hz, 2H), 2.94 (s, 3H), 2.72 (dd, J=8.3, 7.1 Hz, 2H).

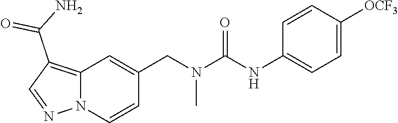 [SMILES: CN(Cc1ccn2ncc(C(N)=O)c2c1)C(=O)Nc1ccc(OC(F)(F)F)cc1]

### 5-((1-methyl-3-(4-(trifluoromethoxy)phenyl)ureido)methyl)pyrazolo[1,5-a]pyridine-3-carboxamide (52)

¹H NMR (600 MHz, CDCl₃) δ 8.41 (d, J=7.8 Hz, 1H), 8.25 (s, 1H), 8.08 (s, 1H), 7.44 (d, J=9.0 Hz, 2H), 7.12 (d, J=9.0 Hz, 2H), 6.92 (m, 1H), 4.65 (s, 2H), 3.05 (s, 3H).

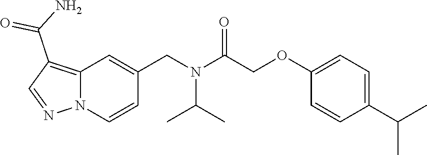 [SMILES: CC(C)c1ccc(OCC(=O)N(Cc2ccn3ncc(C(N)=O)c3c2)C(C)C)cc1]

### 5-((N-isopropyl-2-(4-isopropylphenoxy)acetamido)methyl)pyrazolo[1,5-a]pyridine-3-carboxamide (53)

¹H NMR (600 MHz, CDCl₃) δ 8.25 (bs, 1H), 8.12 (d, J=5.4 Hz, 1H), 8.02 (bs, 1H), 7.10-7.00 (m, 2H), 6.73-6.66 (m, 2H), 6.66-6.61 (m, 1H), 4.76 (bs, 2H), 4.56 (bs, 2H), 4.33 (m, 1H), 2.81-2.78 (m, 1H), 1.16-1.12 (m, 12H).

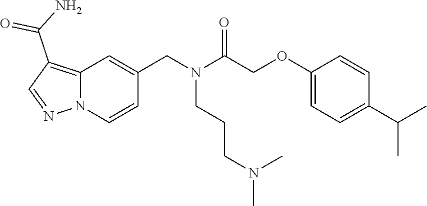 [SMILES: CC(C)c1ccc(OCC(=O)N(CCCN(C)C)Cc2ccn3ncc(C(N)=O)c3c2)cc1]

### 5-((N-(3-(dimethylamino)propyl)-2-(4-isopropylphenoxy)acetamido)methyl)pyrazolo[1,5-a]pyridine-3-carboxamide (54)

¹H NMR (600 MHz, MeOD) δ 8.51 (d, J=7.1 Hz, 1H), 8.34 (s, 1H), 8.01 (s, 1H), 7.07 (d, J=8.6 Hz, 1H), 6.97 (d, J=8.7 Hz, 2H), 6.88 (dd, J=7.2, 2.0 Hz, 1H), 6.84 (d, J=8.8 Hz, 1H), 6.66 (d, J=8.7 Hz, 2H), 4.74 (d, J=2.6 Hz, 4H), 3.47 (t, J=6.7 Hz, 2H), 3.25 (s, 2H), 3.02 (d, J=7.4 Hz, 3H), 2.76-2.74 (m, 1H), 1.94-1.87 (m, 3H), 1.11 (d, J=6.9 Hz, 2H), 1.07 (d, J=6.9 Hz, 6H).

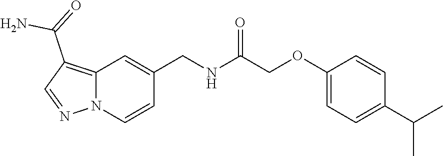 [SMILES: CC(C)c1ccc(OCC(=O)NCc2ccn3ncc(C(N)=O)c3c2)cc1]

### 5-((2-(4-isopropylphenoxy)acetamido)methyl)pyrazolo[1,5-a]pyridine-3-carboxamide (55)

¹H NMR (600 MHz, CDCl₃) δ 8.42 (dd, J=7.1, 0.9 Hz, 1H), 8.20 (s, 1H), 8.15 (s, 1H), 7.16 (d, J=8.7 Hz, 3H), 6.93-6.90 (m, 1H), 6.85 (d, J=8.8 Hz, 2H), 4.62 (d, J=6.3 Hz, 2H), 4.57 (s, 2H), 2.91-2.84 (m, 1H), 1.22 (d, J=6.9 Hz, 6H).

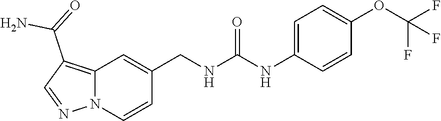 [SMILES: NC(=O)c1cnn2ccc(CNC(=O)Nc3ccc(OC(F)(F)F)cc3)cc12]

### 5-((3-(4-(trifluoromethoxy)phenyl)ureido)methyl)pyrazolo[1,5-a]pyridine-3-carboxamide (56)

¹H NMR (600 MHz, MeOD) δ 8.56 (d, J=7.3 Hz, 1H), 8.41 (s, 1H), 8.18 (dd, J=2.1, 1.1 Hz, 1H), 7.50-7.46 (m, 2H), 7.19-7.15 (m, 2H), 7.05 (dd, J=7.2, 2.0 Hz, 1H), 4.51 (s, 2H).

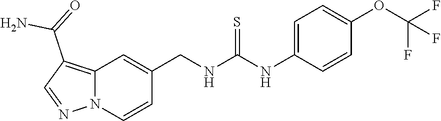 [SMILES: NC(=O)c1cnn2ccc(CNC(=S)Nc3ccc(OC(F)(F)F)cc3)cc12]

### 5-((3-(4-(trifluoromethoxy)phenyl)thioureido)methyl)pyrazolo[1,5-a]pyridine-3-carboxamide (57)

¹H NMR (600 MHz, MeOD) δ 8.55 (d, J=6.9 Hz, 1H), 8.41 (s, 1H), 8.15 (s, 1H), 7.58-7.48 (m, 2H), 7.29 (d, J=8.3 Hz, 2H), 7.13-7.06 (m, 1H), 4.95 (s, 2H).

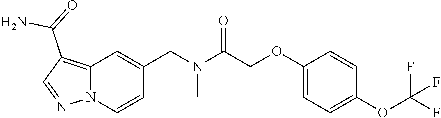 [SMILES: CN(Cc1ccn2ncc(C(N)=O)c2c1)C(=O)COc1ccc(OC(F)(F)F)cc1]

### 5-((N-methyl-2-(4-(trifluoromethoxy)phenoxy)acetamido)methyl)pyrazolo[1,5-a]pyridine-3-carboxamide (58)

¹H NMR (600 MHz, DMSO-d₆) δ 8.71 (d, J=7.2 Hz, 1H), 8.53 (s, 1H), 8.05 (s, 1H), 7.77 (bs, 1H), 7.30 (d, J=8.4 Hz, 2H), 7.09 (bs, 1H), 7.04 (d, J=9.0 Hz, 2H), 6.88 (d, J=7.2 Hz, 1H), 5.02 (s, 2H), 4.62 (s, 2H), 3.03, 2.89 (s, 3H).

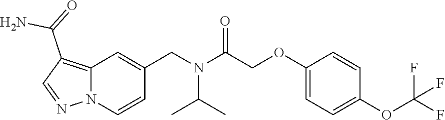 [SMILES: CC(C)N(Cc1ccn2ncc(C(N)=O)c2c1)C(=O)COc1ccc(OC(F)(F)F)cc1]

### 5-((N-isopropyl-2-(4-(trifluoromethoxy)phenoxy)acetamido)methyl)pyrazolo[1,5-a]pyridine-3-carboxamide (59)

¹H NMR (600 MHz, DMSO-d₆) δ 8.63 (d, J=7.2 Hz, 1H), 8.49 (s, 1H), 8.03 (s, 1H), 7.72 (bs, 1H), 7.30 (d, J=9.0 Hz, 2H), 7.03 (d, J=9.0 Hz, 2H), 6.89 (m, 1H), 5.08 (s, 2H), 4.55 (s, 2H), 4.22 (m, 1H), 1.18 (d, J=6.6 Hz, 6H).

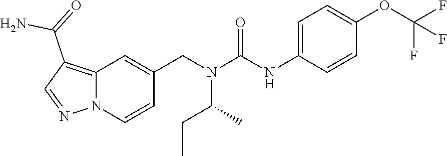 [SMILES: CC[C@@H](C)N(Cc1ccn2ncc(C(N)=O)c2c1)C(=O)Nc1ccc(OC(F)(F)F)cc1]

### (R)-5-((1-(sec-butyl)-3-(4-(trifluoromethoxy)phenyl)ureido)methyl)pyrazolo[1,5-a]pyridine-3-carboxamide (60)

¹H NMR (600 M-z, DMSO-d₆) δ 8.66 (t, J=7.2 Hz, 1H), 8.47 (s, 1H), 8.10 (s, 1H), 7.67 (bs, 1H), 7.51 (d, J=9.0 Hz, 2H), 7.20 (d, J=8.4 Hz, 2H), 6.97 (m, 1H), 4.66 (d, J=18.0 Hz, 1H), 4.46 (d, J=17.4 Hz, 1H), 4.26 (m, 1H), 1.58 (m, 1H), 1.46 (m, 1H), 1.11 (d, J=6.6 Hz, 3H), 0.81 (t, J=7.2 Hz, 3H).

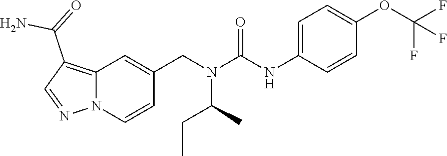 [SMILES: CC[C@H](C)N(Cc1ccn2ncc(C(N)=O)c2c1)C(=O)Nc1ccc(OC(F)(F)F)cc1]

### (S)-5-((1-(sec-butyl)-3-(4-(trifluoromethoxy)phenyl)ureido)methyl)pyrazolo[1,5-a]pyridine-3-carboxamide (61)

¹H NMR (600 MHz, CDCl₃) δ 8.37 (d, J=7.2 Hz, 1H), 8.21 (s. 1H), 8.09 (s, 1H), 7.28 (t, J=7.2 Hz, 2H), 7.03 (d, J=9.0 Hz, 2H), 6.92 (d, J=7.2 Hz, 1H), 4.59 (d, J=17.4 Hz, 1H), 4.38 (d, J=17.4 Hz, 1H), 4.13 (m, 1H), 1.60 (m, 1H), 1.49 (m, 1H), 1.18 (d, J=6.6 Hz, 3H), 0.88 (t, J=7.2 Hz, 3H).

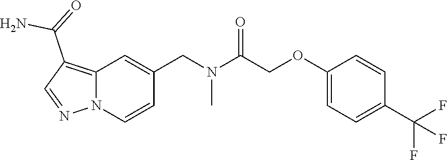 [SMILES: CN(Cc1ccn2ncc(C(N)=O)c2c1)C(=O)COc1ccc(C(F)(F)F)cc1]

### 5-((N-methyl-2-(4-(trifluoromethyl)phenoxy)acetamido)methyl)pyrazolo[1,5-a]pyridine-3-carboxamide (62)

¹H NMR (600 MHz, CDCl₃) δ 8.42 (d, J=7.2 Hz, 1H), 8.23 (s, 1H), 8.17 (s, 1H), 7.59 (d, J=8.4 Hz, 2H), 7.07 (d, J=8.4 Hz, 2H), 6.80 (m, 1H), 6.05 (bs, 2H), 4.89 (s, 2H), 4.70 (s, 2H), 3.10 (s, 3H).

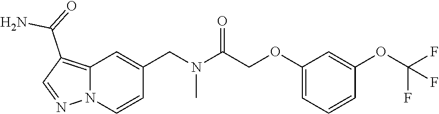 [SMILES: CN(Cc1ccn2ncc(C(N)=O)c2c1)C(=O)COc1cccc(OC(F)(F)F)c1]

### 5-((N-methyl-2-(3-(trifluoromethoxy)phenoxy)acetamido)methyl)pyrazolo[1,5-a]pyridine-3-carboxamide (63)

¹H NMR (600 MHz, CDCl₃) δ 8.32 (d, J=7.2 Hz, 1H), 8.18 (s, 1H), 8.07 (s, 1H), 7.24 (t, J=8.4 Hz, 1H), 6.84 (d, J=8.4 Hz, 2H), 6.81 (m, 2H), 4.73 (m, 2H), 4.62 (s, 2H), 3.00 (s, 3H).

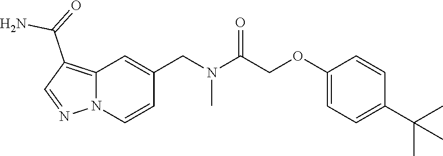 [SMILES: CN(Cc1ccn2ncc(C(N)=O)c2c1)C(=O)COc1ccc(C(C)(C)C)cc1]

### 5-((2-(4-(tert-butyl)phenoxy)-N-methylacetamido)methyl)pyrazolo[1,5-a]pyridine-3-carboxamide (64)

¹H NMR (600 MHz, CDCl₃) δ 8.27 (d, J=7.2 Hz, 1H), 8.16 (s, 1H), 8.05 (s, 1H), 7.24 (d, J=9.0 Hz, 2H), 6.83 (d, J=9.0 Hz, 2H), 6.71 (m, 1H), 4.71 (s, 2H), 4.60 (s, 2H), 2.99 (s, 3H), 1.22 (s, 9H).

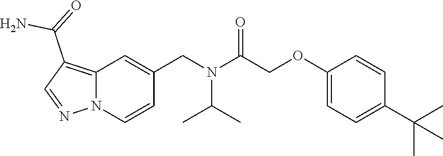 [SMILES: CC(C)N(Cc1ccn2ncc(C(N)=O)c2c1)C(=O)COc1ccc(C(C)(C)C)cc1]

### 5-((2-(4-(tert-butyl)phenoxy)-N-isopropylacetamido)methyl)pyrazolo[1,5-a]pyridine-3-carboxamide (65)

¹H NMR (600 MHz, CDCl₃) δ 8.22 (d, J=7.2 Hz, 1H), 8.07 (s, 1H), 8.02 (s, 1H), 7.26 (d, J=8.4 Hz, 2H), 6.85 (d, J=9.0 Hz, 2H), 6.71 (m, 1H), 4.77 (s, 2H), 4.56 (s, 2H), 4.32 (m, 1H), 1.23 (s, 9H), 1.18 (m, 6H).

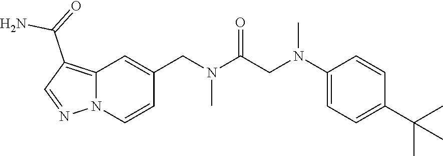 [SMILES: CN(Cc1ccn2ncc(C(N)=O)c2c1)C(=O)CN(C)c1ccc(C(C)(C)C)cc1]

### 5-((2-((4-(tert-butyl)phenyl)(methyl)amino)-N-methylacetamido)methyl)pyrazolo[1,5-a]pyridine-3-carboxamide (66)

¹H NMR (600 MHz, CDCl₃) δ 8.32 (d, J=7.2 Hz, 1H), 8.08 (m, 2H), 7.23 (d, J=8.4 Hz, 2H), 6.77 (d, J=6.0 Hz, 2H), 6.62 (m, 1H), 5.60 (bs, 2H), 4.58 (s, 2H), 4.16 (s, 2H), 3.07 (s, 3H), 2.96 (s, 3H), 1.22 (s, 9H).

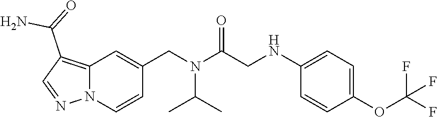 [SMILES: CC(C)N(Cc1ccn2ncc(C(N)=O)c2c1)C(=O)CNc1ccc(OC(F)(F)F)cc1]

### 5-((N-isopropyl-2-((4-(trifluoromethoxy)phenyl)amino)acetamido)methyl)pyrazolo[1,5-a]pyridine-3-carboxamide (67)

¹H NMR (600 MHz, DMSO-d₆) δ 8.63 (d, J=7.2 Hz, 1H), 8.47 (s, 1H), 8.01 (s, 1H), 7.70 (bs, 1H), 7.07 (d, J=8.4 Hz, 2H), 6.91 (d, J=7.8 Hz, 2H), 6.69 (d, J=9.1 Hz, 1H), 5.90 (s, 1H), 4.56 (s, 2H), 4.33 (m, 1H), 4.12 (s, 2H), 1.16 (d, J=6.6 Hz, 6H).

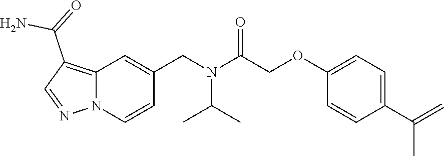 [SMILES: C=C(C)c1ccc(OCC(=O)N(Cc2ccn3ncc(C(N)=O)c3c2)C(C)C)cc1]

### 5-((N-isopropyl-2-(4-(prop-1-en-2-yl)phenoxy)acetamido)methyl)pyrazolo[1,5-a]pyridine-3-carboxamide (68)

¹H NMR (600 MHz, DMSO-d₆) δ 8.73 (d, J=7.2 Hz, 1H), 8.48 (s, 1H), 8.04 (s, 1H), 7.72 (bs, 1H), 6.99 (m, 2H), 6.74 (d, J=9.0 Hz, 1H), 5.01 (s, 2H), 4.98 (d, J=12.0 Hz, 1H), 4.66 (d, J=12.6 Hz, 1H), 4.55 (s. 2H), 4.25 (m, 1H). 2.06 (s, 3H), 1.19 (d, J=6.6 Hz, 6H).

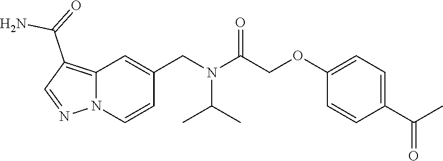 [SMILES: CC(=O)c1ccc(OCC(=O)N(Cc2ccn3ncc(C(N)=O)c3c2)C(C)C)cc1]

### 5-((2-(4-acetylphenoxy)-N-isopropylacetamido)methyl)pyrazolo[1,5-a]pyridine-3-carboxamide (69)

¹H NMR (600 MHz, DMSO-d₆) δ 8.59 (s, 1H), 8.44 (s, 1H), 7.98 (s, 1H), 7.88 (d, J=7.8 Hz, 2H), 7.70 (bs, 1H), 6.98 (d, J=8.4 Hz, 2H), 6.83 (m, 1H), 5.11 (s, 2H), 4.49 (s, 2H), 4.17 (m, 1H), 2.45 (s, 3H), 1.13 (d, J=6.0 Hz, 6H).

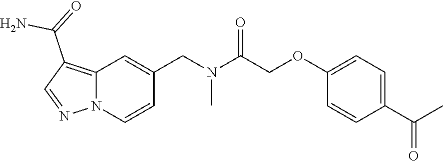 [SMILES: CC(=O)c1ccc(OCC(=O)N(C)Cc2ccn3ncc(C(N)=O)c3c2)cc1]

### 5-((2-(4-acetylphenoxy)-N-methylacetamido)methyl)pyrazolo[1,5-a]pyridine-3-carboxamide (70)

¹H NMR (600 MHz, DMSO-d₆) δ 8.70 (d, J=7.2 Hz, 1H), 8.52 (s, 1H), 8.04 (s, 1H), 7.93 (d, J=9.0 Hz, 2H), 7.77 (bs, 1H), 7.08 (bs, 1H), 7.04 (d, J=9.0 Hz, 2H), 6.88 (m, 1H), 5.10 (s, 2H), 4.62 (s, 2H), 3.04 (s, 3H), 2.52 (s, 3H).

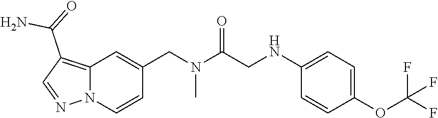 [SMILES: CN(Cc1ccn2ncc(C(N)=O)c2c1)C(=O)CNc1ccc(OC(F)(F)F)cc1]

### 5-((N-methyl-2-((4-(trifluoromethoxy)phenyl)amino)acetamido)methyl)pyrazolo[1,5-a]pyridine-3-carboxamide (71)

¹H NMR (600 MHz, DMSO-d₆) δ 8.68 (d, J=7.2 Hz, 1H), 8.51 (s, 1H), 8.03 (s, 1H), 7.75 (bs, 1H), 7.07 (d, J=8.4 Hz, 2H), 6.83 (d, J=7.2 Hz, 1H), 6.69 (d, J=8.4 Hz, 2H), 5.92 (m, 1H), 4.64 (s, 2H), 4.04 (s, 2H), 3.05 (s, 3H).

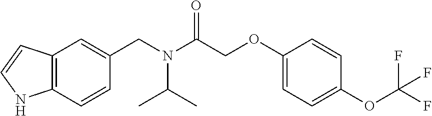 [SMILES: CC(C)N(Cc1ccc2[nH]ccc2c1)C(=O)COc1ccc(OC(F)(F)F)cc1]

### N-((1H-indol-5-yl)methyl)-N-isopropyl-2-(4-(trifluoromethoxy)phenoxy)acetamide (74)

¹H NMR (600 MHz, CDCl₃) δ 8.45 (s, 1H), 7.50 (s, 1H), 7.37 (d, J=8.4 Hz, 1H), 7.22 (s, 1H), 7.18-7.12 (m, 2H), 7.07-7.02 (m, 4H), 6.78 (d, J=8.6 Hz, 2H), 6.51 (s, 1H), 4.61 (s, 2H), 4.57 (s, 2H), 1.20 (d, J=6.8 Hz, 6H).

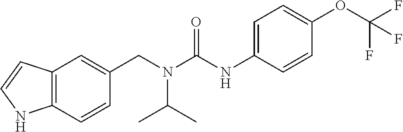 [SMILES: CC(C)N(Cc1ccc2[nH]ccc2c1)C(=O)Nc1ccc(OC(F)(F)F)cc1]

### 1-((1H-indol-5-yl)methyl)-1-isopropyl-3-(4-(trifluoromethoxy)phenyl)urea (75)

¹H NMR (600 MHz, CDCl₃) δ 8.46 (s, 1H), 7.64 (s, 1H), 7.43 (d, J=8.4 Hz, 1H), 7.30-7.24 (m, 1H), 7.17 (dd, J=8.5, 1.7 Hz, 1H), 7.10-7.03 (m, 2H), 6.98 (d, J=8.6 Hz, 2H), 6.51 (d, J=3.1 Hz, 2H), 4.92-4.79 (m, 1H), 4.53 (s, 2H), 1.28 (d, J=6.8 Hz, 6H).

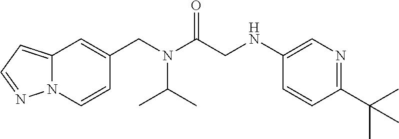 [SMILES: CC(C)N(Cc1ccn2nccc2c1)C(=O)CNc1ccc(C(C)(C)C)nc1]

### 2-((6-(tert-butyl)pyridin-3-yl)amino)-N-isopropyl-N-(pyrazolo[1,5-a]pyridin-5-ylmethyl) acetamide (76)

¹H NMR (600 MHz, CDCl₃) δ 8.39 (d, J=7.2 Hz, 1H), 8.06 (d, J=2.9 Hz, 1H), 7.92 (d, J=2.3 Hz, 1H), 7.34 (s, 1H), 7.20 (d, J=8.6 Hz, 1H), 6.96 (d, J=7.1 Hz, 1H), 6.67 (dd, J=7.2, 1.9 Hz, 1H), 6.43 (d, J=2.2 Hz, 1H), 4.60 (s, 2H), 4.07 (d, J=4.1 Hz, 2H), 1.35 (s, 9H), 1.26 (d, J=6.7 Hz, 6H).

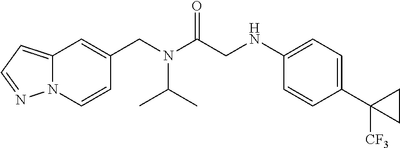 [SMILES: CC(C)N(Cc1ccn2nccc2c1)C(=O)CNc1ccc(C2(C(F)(F)F)CC2)cc1]

### N-isopropyl-N-(pyrazolo[1,5-a]pyridin-5-ylmethyl)-2-((4-(1-(trifluoromethyl)cyclopropyl) phenyl)amino)acetamide (77)

¹H NMR (600 MHz, CDCl₃) δ 8.39 (d, J=7.3 Hz, 1H), 7.91 (d, J=2.3 Hz, 1H), 7.35 (d, J=3.7 Hz, 2H), 7.29 (d, J=8.2 Hz, 2H), 7.19 (d, J=8.1 Hz, 1H), 6.67 (dd, J=7.3, 1.9 Hz, 1H), 6.61 (d, J=8.1 Hz, 2H), 6.49-6.44 (m. 1H), 6.43 (d, J=2.4 Hz, 1H), 4.59 (s, 2H), 4.06 (s, 2H), 1.25 (d, J=6.3 Hz, 6H), 1.19 (d, J=6.8 Hz, 2H), 0.93 (d, J=6.7 Hz, 2H).

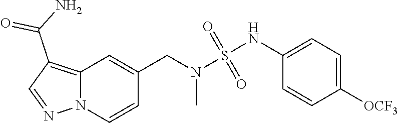 [SMILES: CN(Cc1ccn2ncc(C(N)=O)c2c1)S(=O)(=O)Nc1ccc(OC(F)(F)F)cc1]

### 5-((methyl(N-(4-(trifluoromethoxy)phenyl)sulfamoyl)amino)methyl)pyrazolo[1,5-a]pyridine-3-carboxamide (78)

¹H NMR (600 MHz, DMSO-d₆) δ 8.64 (d, J=7.2 Hz, 1H), 8.50 (s, 1H), 8.10 (s, 1H), 7.73 (bs, 1H), 7.30 (m, 4H), 6.83 (m, 1H), 4.36 (s, 2H), 2.68 (s, 3H).

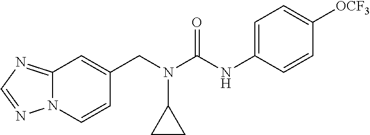 [SMILES: O=C(Nc1ccc(OC(F)(F)F)cc1)N(Cc1ccn2ncnc2c1)C1CC1]

### 1-([1,2,4]triazolo[1,5-a]pyridin-7-ylmethyl)-1-cyclopropyl-3-(4-(trifluoromethoxy)phenyl)urea (79)

¹H NMR (600 MHz, CDCl₃) δ 8.55 (d, J=7.2 Hz, 1H), 8.36 (s, 1H), 7.67 (s, 1H), 7.51 (d, J=8.4 Hz, 2H), 7.47 (s, 1H), 7.20 (d, J=8.4 Hz, 2H), 7.11-7.10 (m, 1H), 4.75 (s, 2H), 2.67 (m, 1H), 1.05 (d, J=4.8 Hz, 2H), 0.97 (d, J=3.0 Hz, 2H).

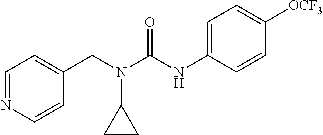 [SMILES: O=C(Nc1ccc(OC(F)(F)F)cc1)N(Cc1ccncc1)C1CC1]

### 1-cyclopropyl-1-(pyridin-4-ylmethyl)-3-(4-(trifluoromethoxy)phenyl)urea (80)

¹H NMR (600 MHz, CDCl₃) δ 8.57 (d, J=6.0 Hz, 2H), 7.51 (d, J=9.0 Hz, 2H), 7.47 (s, 1H), 7.25 (d, J=6.0 Hz, 2H), 7.19 (d, J=8.4 Hz, 2H), 4.65 (s, 2H), 2.67 (m, 1H), 1.02 (d, J=4.8 Hz, 2H), 0.93 (d, J=3.0 Hz, 2H).

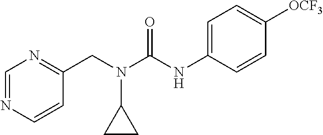 [SMILES: O=C(Nc1ccc(OC(F)(F)F)cc1)N(Cc1ccncn1)C1CC1]

### 1-cyclopropyl-1-(pyrimidin-4-ylmethyl)-3-(4-(trifluoromethoxy)phenyl)urea (81)

¹H NMR (600 MHz, CDCl₃) δ 9.08 (s, 1H), 8.60 (d, J=4.8 Hz, 1H), 7.47 (s, 1H), 7.41 (d, J=8.4 Hz, 2H), 7.23 (s, 1H), 7.09 (d, J=9.0 Hz, 2H), 4.62 (s, 2H), 2.82 (m, 1H), 0.95 (d, J=4.8 Hz, 2H), 0.89 (d, J=3.6 Hz, 2H).

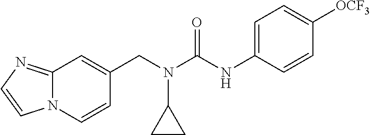 [SMILES: O=C(Nc1ccc(OC(F)(F)F)cc1)N(Cc1ccn2ccnc2c1)C1CC1]

### 1-cyclopropyl-1-(imidazo[1,2-a]pyridin-7-ylmethyl)-3-(4-(trifluoromethoxy)phenyl)urea (82)

¹H NMR (600 MHz, CDCl₃) δ 8.06 (s, 1H), 7.58 (s, 1H), 7.53 (d, J=1.2 Hz, 1H), 7.46 (d, J=6.6 Hz, 2H), 7.43 (s, 1H), 7.13 (d, J=6.6 Hz, 2H), 6.82 (m, 1H), 4.61 (s, 2H), 2.60 (m, 1H), 0.96 (m, 2H), 0.90 (t, J=2.4 Hz, 2H)

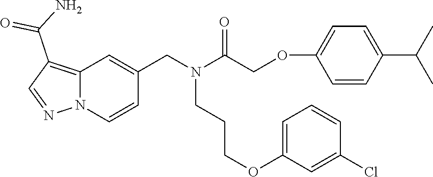 [SMILES: CC(C)c1ccc(OCC(=O)N(CCCOc2cccc(Cl)c2)Cc2ccn3ncc(C(N)=O)c3c2)cc1]

### 5-((N-(3-(3-chlorophenoxy)propyl)-2-(4-isopropylphenoxy)acetamido)methyl)pyrazolo[1,5-a]pyridine-3-carboxamide (83)

¹H NMR (600 MHz, CDCl₃) δ 8.37, 8.30 (m, 1H), 8.14 (s, 1H), 8.08 (d, J=7.2 Hz, 1H), 7.09 (m, 3H), 6.86-6.70 (m, 5H), 6.63 (m, 1H), 5.99 (m, 2H), 4.73 (s, 2H), 4.67 (t, J=7.2 Hz, 2H), 3.88-3.79 (m, 2H), 3.54-3.47 (m, 2H), 2.78 (m 1H), 2.03-1.97 (m, 2H), 1.17-1.13 (m, 6H).

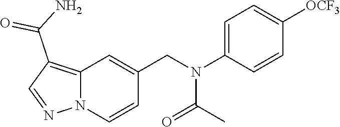 [SMILES: CC(=O)N(Cc1ccn2ncc(C(N)=O)c2c1)c1ccc(OC(F)(F)F)cc1]

### 5-((N-(4-(trifluoromethoxy)phenyl)acetamido)methyl)pyrazolo[1,5-a]pyridine-3-carboxamide (84)

¹H NMR (600 MHz, CDCl₃) δ 8.38 (d, J=7.2 Hz, 1H), 8.08 (s. 1H), 7.94 (s, 1H), 7.13 (d, J=8.4 Hz, 2H), 7.04 (d, J=8.4 Hz, 2H), 6.96 (t, J=6.0 Hz, 1H), 5.68 (bs, 2H), 4.90 (s, 2H), 1.88 (s, 3H).

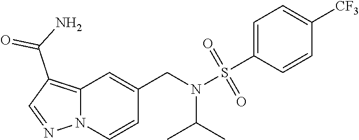 [SMILES: CC(C)N(Cc1ccn2ncc(C(N)=O)c2c1)S(=O)(=O)c1ccc(C(F)(F)F)cc1]

### 5-(((N-isopropyl-4-(trifluoromethyl)phenyl)sulfonamido)methyl)pyrazolo[1,5-a]pyridine-3-carboxamide (85)

¹H NMR (600 MHz, CDCl₃) δ 8.41 (m, 1H), 8.15 (d, J=8.4 Hz, 1H), 8.09 (d, J=12.0 Hz, 1H), 7.90 (d, J=7.8 Hz, 2H), 7.71 (m, 2H), 7.29 (d, J=8.4 Hz, 1H), 7.14 (m, 1H), 4.40 (d, J=12.6 Hz, 2H), 4.16 (m, 1H), 0.92 (m, 6H).

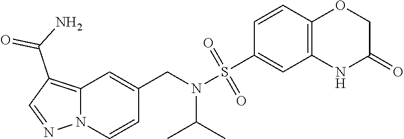 [SMILES: CC(C)N(Cc1ccn2ncc(C(N)=O)c2c1)S(=O)(=O)c1ccc2c(c1)NC(=O)CO2]

### 5-(((N-isopropyl-3-oxo-3,4-dihydro-2H-benzo[b][1,4]oxazine)-6-sulfonamido) methyl) pyrazolo[1,5-a]pyridine-3-carboxamide (86)

¹H NMR (600 MHz, CDCl₃) δ 8.37 (m, 2H), 8.23 (s, 1H). 7.77 (s, 1H), 7.51 (m, 1H), 6.97 (m, 1H), 6.90 (m, 1H), 3.81 (m, 1H), 3.78 (m, 111), 3.07 (m, 1H), 1.01 (d, J=6.6 Hz, 6H).

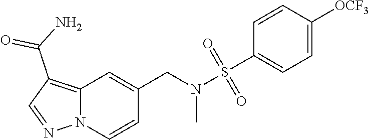 [SMILES: CN(Cc1ccn2ncc(C(N)=O)c2c1)S(=O)(=O)c1ccc(OC(F)(F)F)cc1]

### 5-(((N-methyl-4-(trifluoromethoxy)phenyl)sulfonamido)methyl)pyrazolo[1,5-a]pyridine-3-carboxamide (87)

¹H NMR (600 MHz, CDCl₃) δ 8.43 (d, J=7.2 Hz, 1H), 8.10 (s. 2H), 7.84 (d, J=9.0 Hz, 2H), 7.35 (d, J=7.8 Hz, 2H), 7.08 (m, 1H), 5.66 (bs, 2H), 4.18 (s, 2H), 2.61 (s, 3H).

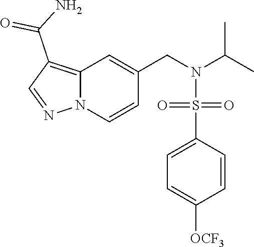 [SMILES: CC(C)N(Cc1ccn2ncc(C(N)=O)c2c1)S(=O)(=O)c1ccc(OC(F)(F)F)cc1]

### 5-(((N-isopropyl-4-(trifluoromethoxy)phenyl)sulfonamido)methyl)pyrazolo[1,5-a]pyridine-3-carboxamide (88)

¹H NMR (600 MHz, CDCl₃) δ 8.41 (d, J=7.2 Hz, 11H), 8.12 (d, J=9.6 Hz, 2H), 7.83 (d, J=9.0 Hz, 2H), 7.28 (d, J=8.4 Hz, 2H), 7.18 (m, 1H), 5.73 (bs, 2H), 4.40 (s, 2H), 4.14 (m, 111), 0.93 (d, J=7.2 Hz, 6H).

### Example 5: Validation of NTRK (NTRK1, NTRK2, and/or NTRK3) Activity of Selected Compounds

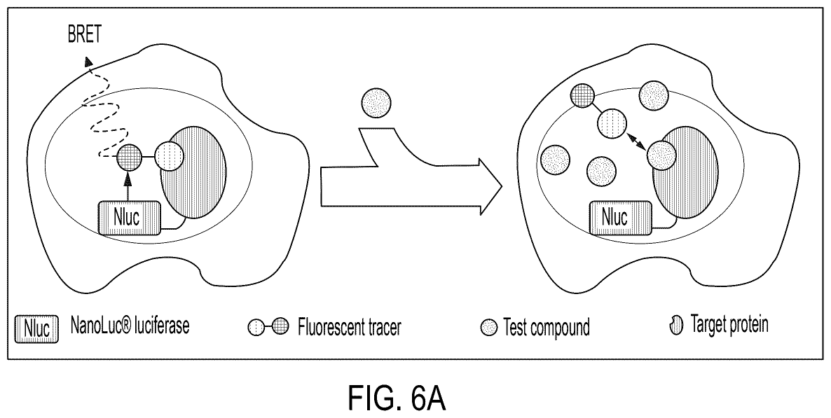

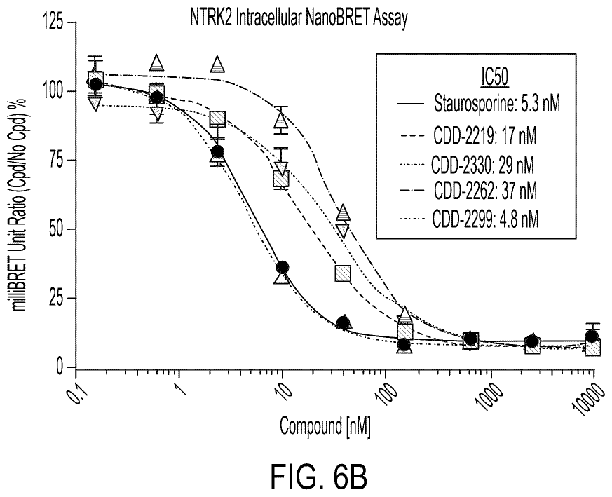

As described elsewhere herein, hit compounds, including but not limited to CDD-2262, and its smaller analog, CDD-2330, were synthesized off-DNA and evaluated for NTRK2 activity. Selected compounds (i.e., CDD-2299, CDD-2219, CDD-2330, CDD-2262, CDD-2737, and CDD-3032, inter alia) showed low nanomolar potency (IC₅₀ of 3.7, 13, 3.5, 32, 2.3, and 6.1 nM, respectively; see Table 2) and were further able to penetrate cell membranes to induce an effect in NanoBRET assays (FIGS. 6A-6B).

The inhibitory properties of select compounds of the present disclosure were analyzed against NTRK1, NTRK2, and NTRK3 (Table 2). Further, the apparent inhibition constant (Kᵢ^{app}) was determined for select compounds against NTRK2 (Table 3). Compounds with an IC₅₀≥10,000 nM are considered inactive herein.

TABLE 2
Exemplary NTRK2 inhibitory data for certain compounds

| | NTRK1 | NTRK2 | NTRK3 |
|---|---|---|---|
| Compound | IC₅₀ (nM) | IC₅₀ (nM) | IC₅₀ (nM) |
| CDD-2330 | 3.8 | 3.5 | 1.2 |
| CDD-2301 | — | 513 | — |
| CDD-2300 | — | >10,000 | — |
| CDD-2299 | 31 | 3.7 | — |
| CDD-2298 | — | 99 | — |
| CDD-2297 | — | >10,000 | — |
| CDD-2296 | — | 335 | — |
| CDD-2276 | — | 19 | — |
| CDD-2275 | — | 46 | — |
| CDD-2274 | 12.4 | 4.5 | 5.3 |
| CDD-2273 | — | 1317 | — |
| CDD-2262 | — | 32 | — |
| CDD-2222 | — | 43 | — |
| CDD-2219 | — | 13 | — |
| CDD-2734 | 36 | 5.3 | — |
| CDD-2735 | 4.3 | 3.6 | 4.4 |
| CDD-2736 | 4.1 | 2.5 | 3.3 |
| CDD-2737 | 6.1 | 2.3 | 3.1 |
| CDD-2738 | 7.5 | 0.52 | 1.51 |
| CDD-2741 | 11.7 | 5.8 | 4.4 |
| CDD-2742 | 104 | 5.7 | 11.8 |
| CDD-2743 | 9.5 | 6 | 5.4 |
| CDD-2744 | 18.1 | 1.7 | 2.9 |
| CDD-3118 | 151 | 19 | 21.5 |
| CDD-3119 | 3.8 | 3.6 | 6.6 |
| CDD-3120 | 1779 | 242 | |
| CDD-3121 | 11588 | 3236 | |
| CDD-3031 | 153 | 11.9 | 25.3 |
| CDD-3032 | 34 | 6.1 | 13.7 |
| CDD-3129 | 9541 | 3765 | |
| CDD-3130 | 9197 | 1265 | |
| CDD-3131 | 749 | 132 | 153 |
| CDD-3132 | 4559 | 517 | 1372 |
| CDD-3133 | 16.6 | 4.3 | 10.1 |
| CDD-3057 | 17.6 | 4 | 6.6 |
| CDD-3058 | 11.7 | 3.7 | 8.4 |
| CDD-3099 | 39611 | 47392 | |
| CDD-3100 | 26276 | 37240 | |
| CDD-3181 | 6717 | 1065 | |
| CDD-3182 | 4057 | 354 | |
| CDD-3183 | 3610 | 247 | |
| CDD-3184 | 61 | 6.7 | 15.3 |
| CDD-3185 | 10914 | 1753 | |
| CDD-3254 | 277 | 59 | 142 |
| CDD-3255 | 357 | 31.5 | 54 |
| CDD-3256 | 1870 | 223 | 312 |
| CDD-3257 | 58163 | 10598 | 10917 |
| CDD-3258 | 10986 | 963 | 2318 |
| CDD-3264 | 43881 | 2092 | 21777 |
| CDD-3265 | 50113 | 53743 | 55086 |
| CDD-3306 | 2408 | 1130 | 4770 |
| CDD-3307 | 2169 | 414 | 922 |
| CDD-3308 | 28431 | 4445 | 4317 |
| Staurosporine | 8.3 | 10 | 6.7 |
| Entrectinib | 2.8 | 4.5 | 11.1 |
| Larotrectinb | 46.6 | 61.2 | 22.5 |
| Selitrectinib | 8.2 | 6.5 | 3.4 |
| Reprotrectinib | 7 | 4.3 | 3.6 |
| PF-06273340 | 17.8 | 8.3 | 18.3 |

TABLE 3
NTRK2 Apparent Inhibition Constant
(Kᵢ^{app}) data for select compounds

| Compound | NTRK2 Kᵢ^{app} (nM) |
|---|---|
| CDD-2262 | 17 ± 10 |
| CDD-2222 | 34 ± 10 |
| CDD-2219 | 21 ± 7 |

### Example 6: Validation of NTRK2 Specificity for CDD-2330

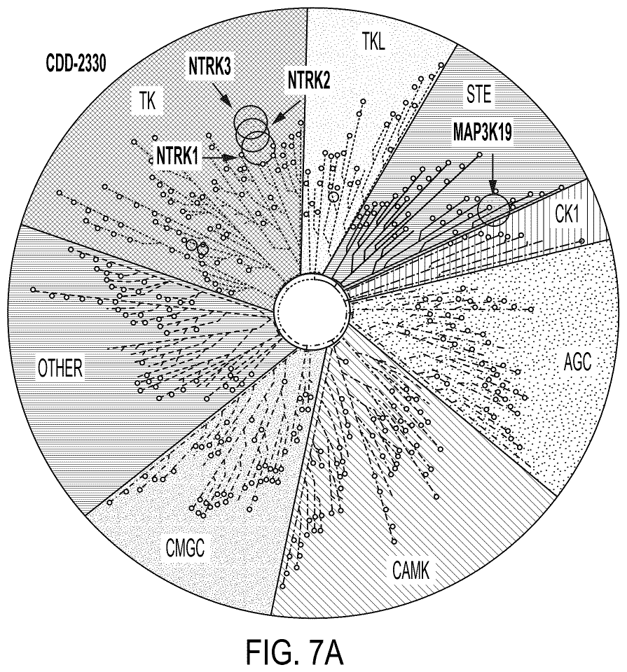

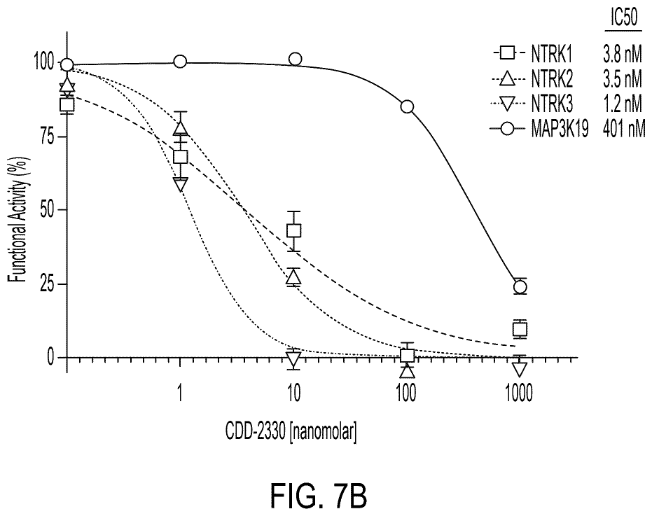

To broadly assess the kinome-wide selectivity of CDD-2330, a KINOMEscan profiling platform was employed (FIG. 7A). At a concentration of 1 μM, CDD-2330 showed high specificity for NTRKs, with MAP3K19 as the only possible off-target. Further dose response evaluation of CDD-2330 in the ThermoFisher Z'-LYTE assay (FIG. 7B) confirmed that CDD-2330 binds NTRKs in the range of single-digit nanomolar activities (IC₅₀: 1.2-3.8 nM) and shows >100-fold selectivity over MAP3K19 (IC₅₂: 401 nM).

Similarly, the selectivity of CDD-3031 for NTRK kinases was evaluated in a kinase occupancy assay (Promega K192 cell-based NanoBRET assay) using 192 kinases (Table 4). CDD-3031 was delivered at 1 μM in the assay and showed greater selectivity for NTRK2 and NTRK1, confirming the findings in Table 2.

TABLE 4
CDD-3031 kinase occupancy (%)

| Kinase | Average (%) | Replicate 1 (%) | Replicate 2 (%) |
|---|---|---|---|
| NTRK2 | 92.92 | 94.61 | 91.24 |
| NTRK1 | 73.22 | 71.41 | 75.03 |
| MUSK | 19.82 | 17.46 | 22.19 |
| TIE1 | 14.02 | 8.54 | 19.49 |
| MAP3K19 | 13.32 | 53.26 | −26.61 |
| TEK | 10.20 | 13.06 | 7.34 |
| JAK2 (V617F) | 9.92 | 31.45 | −11.62 |
| CDKL1 | 8.23 | 2.80 | 13.66 |
| MAP3K12 | 7.58 | 21.20 | −6.05 |
| AURKB | 7.37 | 3.71 | 11.04 |
| FLT3 | 7.12 | −10.34 | 24.58 |
| DYRK1A | 6.50 | 8.58 | 4.42 |
| STK11 | 5.85 | 6.96 | 4.74 |
| ITK | 5.61 | 14.16 | −2.94 |
| NEK5 | 5.02 | −0.81 | 10.85 |
| WEE1 | 4.94 | 9.23 | 0.65 |
| CLK1 | 4.92 | 3.65 | 6.20 |
| STK36 | 4.82 | −3.75 | 13.40 |
| MKNK2 | 4.61 | 7.96 | 1.26 |
| CSNK1A1L | 4.58 | 9.24 | −0.08 |
| ULK2 | 4.48 | −5.12 | 14.09 |
| SGK1 | 4.31 | 1.93 | 6.69 |
| PKMYT1 | 4.17 | 5.47 | 2.88 |
| AURKC | 4.09 | −7.48 | 15.66 |

### Example 7: Pharmacokinetic Data for Compounds of the Present Disclosure

Previous data has indicated the risk of on-target adverse events associated, with both NTRK1 and NTRK2, resulting from pharmacologically relevant exposure in the central nervous system. Both FDA approved small molecule pan-NTRK inhibitors (i.e., entrectinib and larotrectinib) have been shown to cross the blood-brain barrier, which is considered a major concern for the treatment of endometriosis, a non-life-threatening condition. Thus, in certain non-limiting embodiments, a peripherally restricted NTRK1-specific inhibitor with a new chemotype is a safer alternative and has great potential for the treatment of bot inflammation and pain associated with endometriosis.

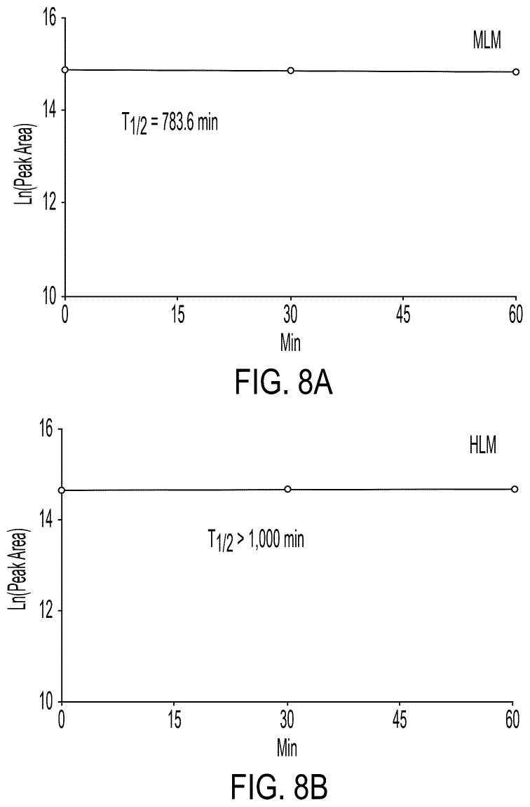

Using mouse and human liver microsomes in vitro, CDD-2262 was shown to be a metabolically stable compound (t_{1/2}>7 hr), while analog CDD-2330 was found to be even more stable (t_{1/2}>25 hr) (Table 5). Certain pharmacokinetic properties (e.g., clearance) of exemplary compound CDD-3031 are further provided herein (Tables 6-7 and FIGS. 8A-8B).

Taken together, the data indicate that the compounds described herein are potent, metabolically stable, and NTRK1-specific compounds.

TABLE 5
Half-life in mouse liver microsomes (MLM) and human liver microsomes (HLM)

| Compound | MLM^{a} t_{1/2} (min) | HLM^{b} t_{1/2} (min) | Compound | MLM t_{1/2} (min) | HLM t_{1/2} (min) |
|---|---|---|---|---|---|
| JQ1 | 12 | 9.5 | CDD-2741 | 41 | 128 |
| Alprazolam | 350 | 731 | CDD-2744 | 121 | 388 |
| Selitrectinib | 32 | 244 | CDD-3031 | 784 | >1000 |
| Reprotrectinib | 167 | 254 | CDD-3032 | 6.3 | 28.3 |
| PF-06273340 | 202 | 502 | CDD-3118 | 688 | 382 |
| CDD-2330 | 1635 | 1538 | CDD-3119 | 290 | 96 |
| CDD-2299 | 20.2 | 43.1 | CDD-3131 | 351 | 237 |
| CDD-2276 | 35 | 14 | CDD-3132 | 706 | 691 |
| CDD-2275 | 56 | 90 | CDD-3133 | 185 | 602 |
| CDD-2274 | 5 | 11.5 | CDD-3184 | 6.1 | 107 |
| CDD-2273 | 8.2 | 15.5 | CDD-3254 | 143 | 193 |
| CDD-2262 | 532 | 442 | CDD-3255 | 19 | 45 |
| CDD-2219 | 28.2 | 54.8 | CDD-3256 | >200 | >200 |
| CDD-2734 | 21 | 58 | CDD-3257 | >200 | >200 |
| CDD-2737 | 156 | 190 | CDD-3258 | 338 | 270 |
| CDD-2738 | 224 | 381 | | | |

TABLE 6
Exemplary clearance data for CDD-3031

| Mouse Liver Microsomes (MLM) | Human Liver Microsomes (HLM) |
|---|---|
| CLᵢₙₜ (μL/min/mg) | CLᵢₙₜ (μL/min/mg) |
| 1.8 | 0.0 |

TABLE 7
Exemplary pharmacokinetic data for CDD-
3031 (n = 3, mean ± SD)

| Parameter | Unit | CDD-3031 |
|---|---|---|
| Tₘₐₓ | hr | 1.17 ± 0.76 |
| Cₘₐₓ | μmol/L | 37.10 ± 4.88 |
| t_{1/2} | hr | 8.03 ± 1.25 |
| AUC₀₋ₜ | hr*μmol/L | 418.29 ± 62.11 |
| AUC_{0-∞} | hr*μmol/L | 478.43 ± 87.78 |
| CL/F | mg/(hr*μmol/L)/kg | 0.11 ± 0.02 |
| Vd/F | mg/(μmol/L)/kg | 1.22 ± 0.05 |
| MRT₀₋ₜ | hr | 7.23 ± 0.38 |
| MRT_{0-∞} | hr | 10.71 ± 1.52 |

CDD-2330 has a low brain uptake (BrainzPlasma is 0.31 at 4 h) compared to

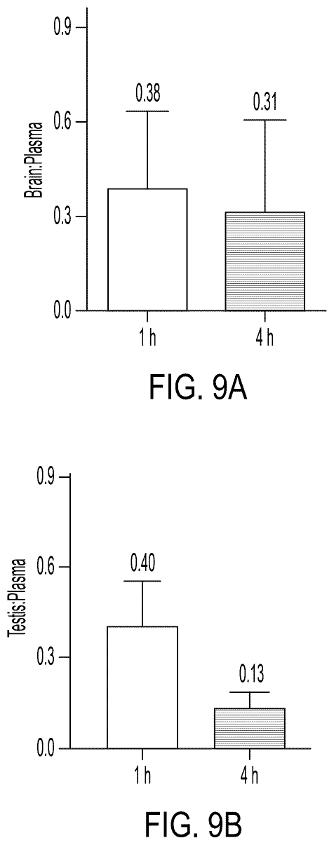

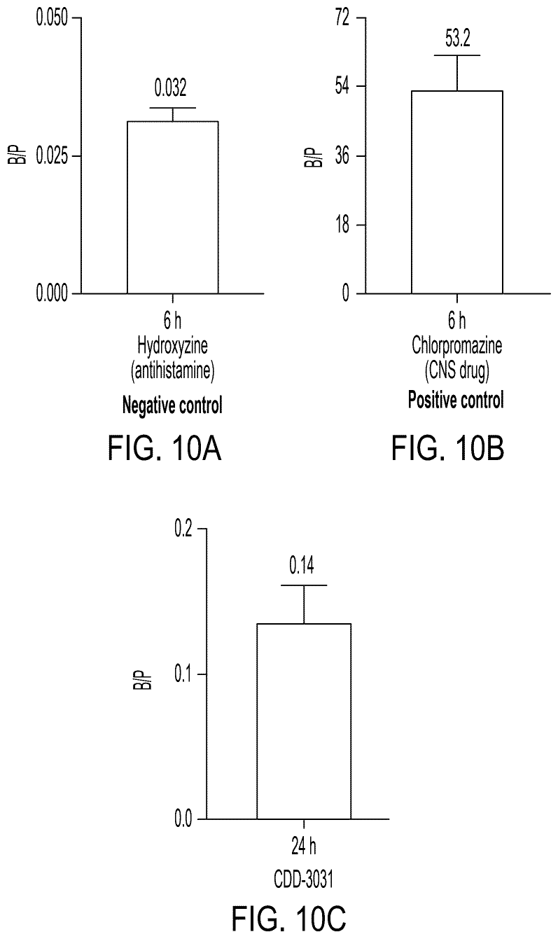

antidepressant drugs such as atomoxetine (Brain:Plasma is 20 at 4 h) (FIG. 9A), and also has low testis uptake (Testis:Plasma is 0.31 at 4 h) (FIG. 9B). Similarly, CDD-3031 has a relatively low brain uptake compared to chlorpromazine (positive control), but a high uptake as compared to hydroxyzine (negative control) (FIGS. 10A-10C).

TABLE 8
Exemplary brain uptake data for certain compounds

| Compound | Brain Concentration at 24 h (ng/mg) |
|---|---|
| PF-06273340 | 0.08 |
| CDD-3119 | 0.14 |
| CDD-2737 | 0.04 |
| CDD-3031 | 0.35 |
| CDD-2738 | 2.3 |

Additionally, analysis of potential liver and other tissue toxicity after a high dose of certain exemplary compounds, including CDD-2330 and CDD-3031 (i.e., 50 mg/kg), indicates minimal effects (Tables 9-10).

TABLE 9
Evaluation of potential liver and other tissue toxicity
in mice treated with a high dose of CDD-2330^{a}

| Mouse No. | Unit | ALT | AST |
|---|---|---|---|
| 1 | U/L | 13.97 | 19.45 |
| 2 | U/L | 26.17 | 18.92 |
| 3 | U/L | 13.44 | 26.17 |
| 4 | U/L | 18.74 | 13.26 |
| Control | U/L | 11.67 | 15.03 |

^{a}Plasma ALT and AST levels were evaluated 48 h after mice (n = 4) were orally administered 50 mg/kg CDD-2330

TABLE 10
Evaluation of potential liver and other tissue toxicity
in mice treated with a high dose of CDD-3031^{a}

| Mouse No. | Unit | ALT | AST |
|---|---|---|---|
| 1 | IU/L | 35.89 | 86.81 |
| 2 | IU/L | 24.49 | 64.53 |
| 3 | IU/L | 52.86 | 68.33 |
| 4 | IU/L | 48.18 | 72.84 |
| Control-1 | IU/L | 34.56 | 41.81 |
| Control-2 | IU/L | 39.87 | 48.71 |

^{a}Plasma ALT and AST levels were evaluated 24 h after mice were administered 50 mg/kg CDD-3031 by oral gavage

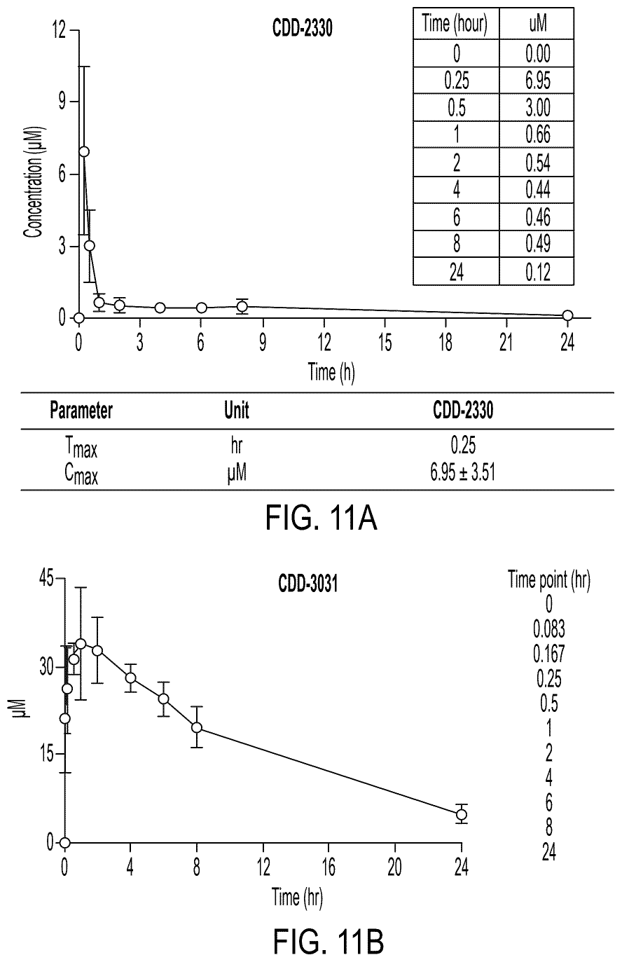

Further, initial pharmacokinetics analysis of orally administered CDD-2330 indicates rapid disappearance in the blood, which may be at least partially attributed to metabolism (FIG. 11A). Thus, the present disclosure indicates that CDD-2330, and analogues thereof, may be safer peripherally restricted pan-NTRK inhibitors for the treatment of endometriosis and multiple cancers.

In certain embodiments, compounds of the present invention comprising a carboxyl group at the 3-position of the pyrazolo[1,5-a]pyridine scaffold possess potent NTRK1, NTRK2, and/or NTRK3 activity. In certain embodiments, substitution of the carboxy functionality at the 3-position of the pyrazolo[1,5-a]pyridine scaffold with any of a number of substituents, including but not limited to halogens, amidines, N-hydroxyamidines, N-hydroxyamides (e.g., Weinreb amides), improves the pharmacokinetic properties of the compound. Without wishing to be bound by theory, substitution of the carboxylic acid group with any of a number of substituents is thought to improve the pharmacokinetics of the compounds by reduction of glucuronidation.

### Example 8: CDD-2330 Administration Results in Downregulation of Markers Associated with Inflammation in Endometriosis Cells

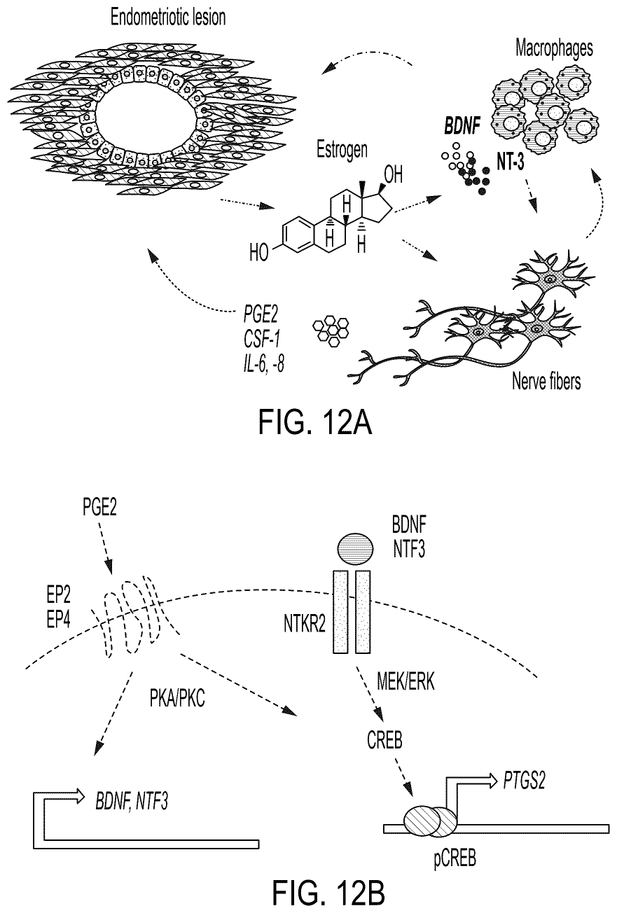

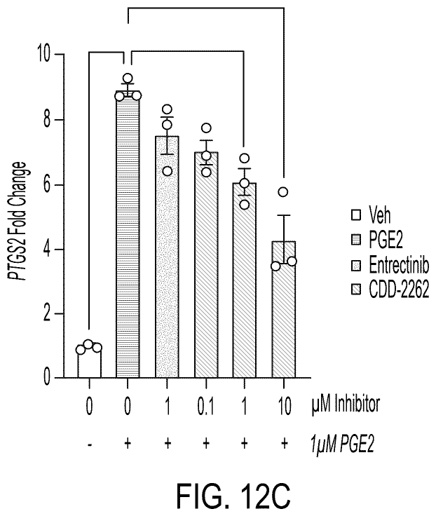

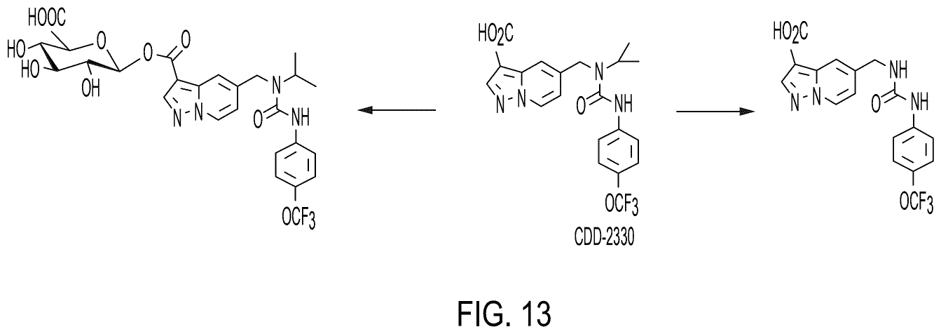

Estrogen produced by the ovary and endometriotic lesions, is a key molecule that induces BDNF/NT3 secretion from macrophages and pro-inflammatory signaling from nerve fibers (FIG. 12A). This signaling mechanism exacerbates the pro-inflammatory state of the endometriotic lesion and promotes nerve fiber outgrowth and pain. At the cellular level, it has been determined that PGE2 and BDNF/NT-3 signaling stimulate PTGS2 expression via phosphorylation and activation of CREB (FIG. 12B). Furthermore, inhibition of NTRK2 with SiRNAs attenuates pro-inflammatory gene expression.

Preliminary studies with CDD-2262 (i.e., CDD-2330 analog) demonstrate that inhibition of NTRK2 statistically suppresses prostaglandin-endoperoxidase synthase 2 (PTGS2) mRNA over a 6-hour period at a concentration >100 nM (FIG. 12C). Thus, the present disclosure provides evidence that administration of CDD-2330 results in suppression of markers associated with inflammation, and accordingly, is suitable for the treatment of endometriosis.

### Example 9: Exemplary Data for Certain NTRK2 Inhibitors in a Mouse Model of Endometriosis

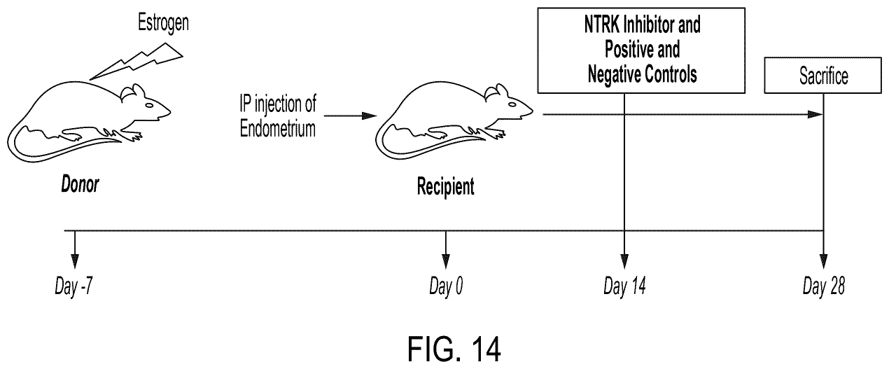

In one aspect, the present disclosure provides exemplary data relating to the administration of NTRK2 inhibitors in a mouse model of endometriosis (FIG. 14). In certain embodiments, the recipient mouse is administered the NTRK2 inhibitor and/or controls on day 14 after intraperitoneal injection of endometrium of the donor mouse. In certain embodiments, the recipient mouse is sacrificed and quantitatively assessed on day 28.

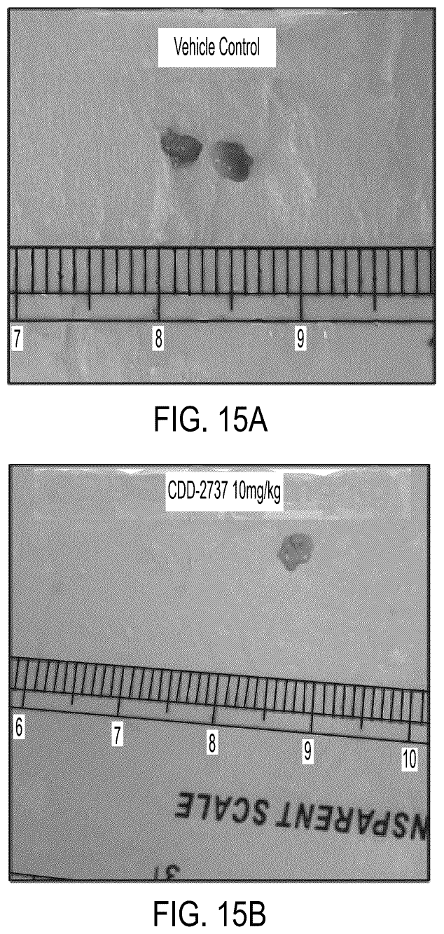

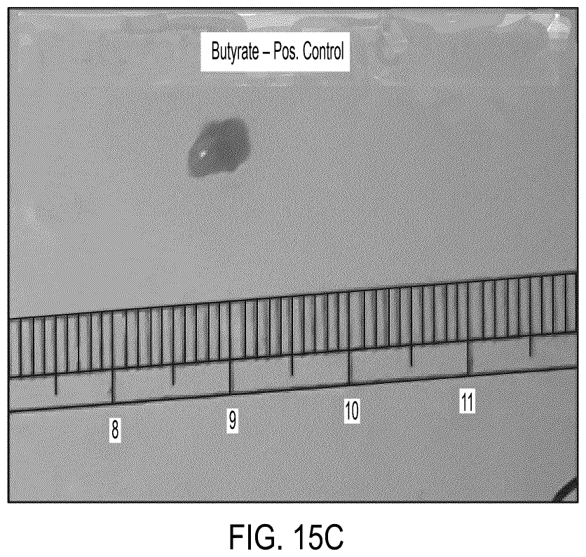

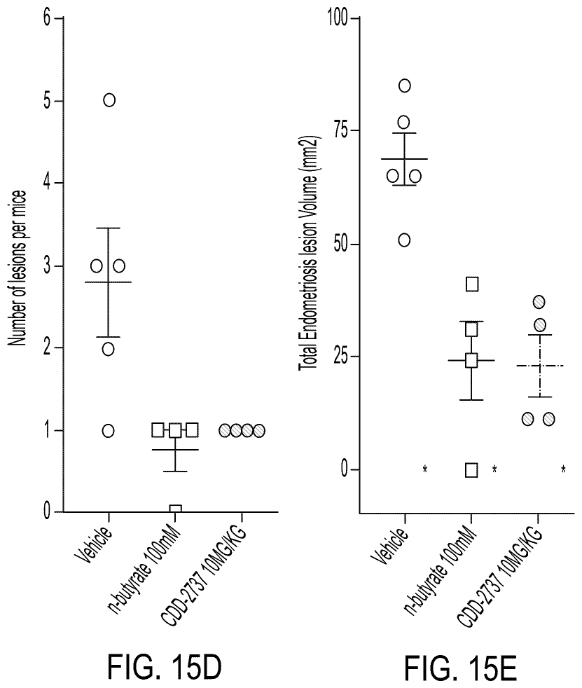

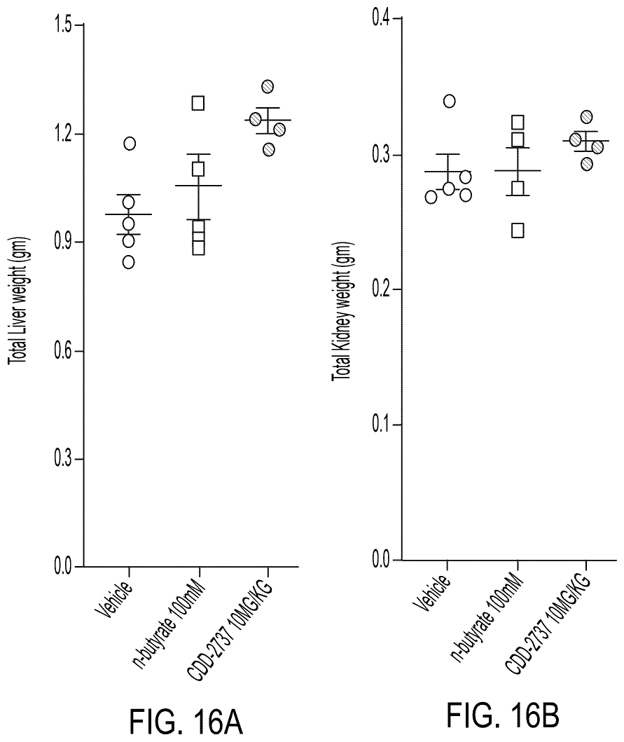

The present disclosure provides exemplary data demonstrating that administration of CDD-2737 to an endometriosis mouse model results in a decreased number of lesions and lesions of diminished size, as compared to controls (FIGS. 15A-15E). Further, administration of CDD-2737 showed no gross effects on the liver and/or kidney (FIGS. 16A-16B) or potential liver or other tissue toxicity (Table 11).

TABLE 11
Evaluation of potential liver and other tissue toxicity
in mice treated with CDD-2737 at 10 mg/kg/day for 14 days

| | Mouse # | ALT (U/L) | AST (U/L) |
|---|---|---|---|
| Control | C350 | 34.74 | 32.35 |
| | C351 | 21.48 | 37.75 |
| | C352 | 32.18 | 30.06 |
| | C353 | 20.16 | 31.03 |
| | C354 | 22.28 | 76.11 |
| Average | | 26.2 | 41.5 |
| n-butyrate 100 mM | C371 | 11.14 | 31.03 |
| | C372 | 16.53 | 70.54 |
| | C373 | 36.33 | 39.25 |
| | C374 | 27.58 | 51.01 |
| Average | | 22.9 | 48.0 |
| CDD-2737 | C355 | 13.79 | 28.11 |
| | C356 | 13.61 | 47.21 |
| | C357 | 10.25 | 27.58 |
| | C358 | 23.16 | 22.28 |
| Average | | 15.2 | 31.3 |

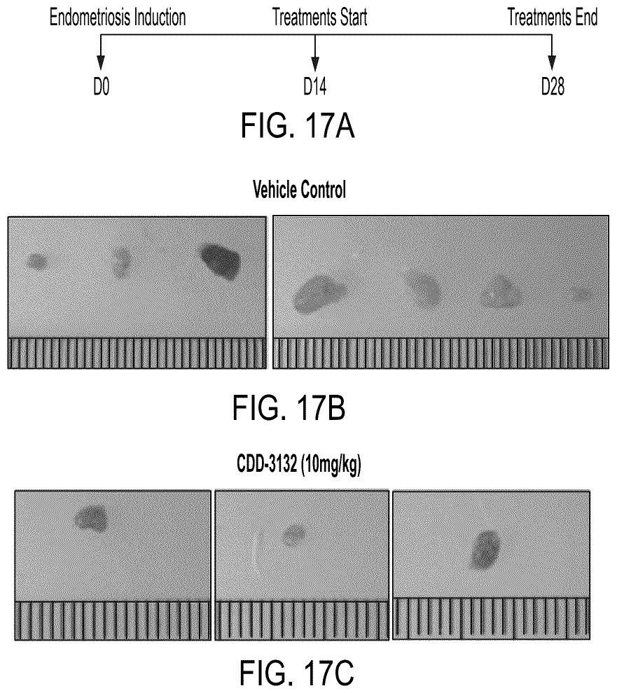

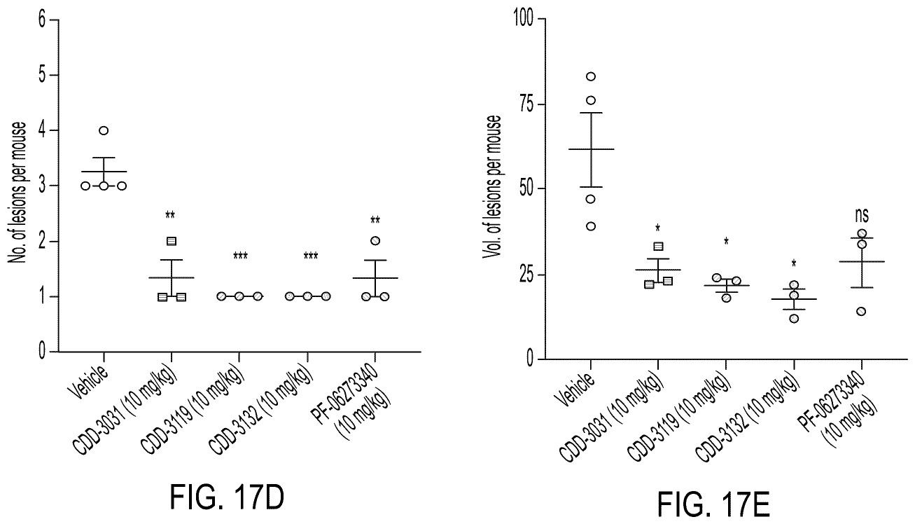

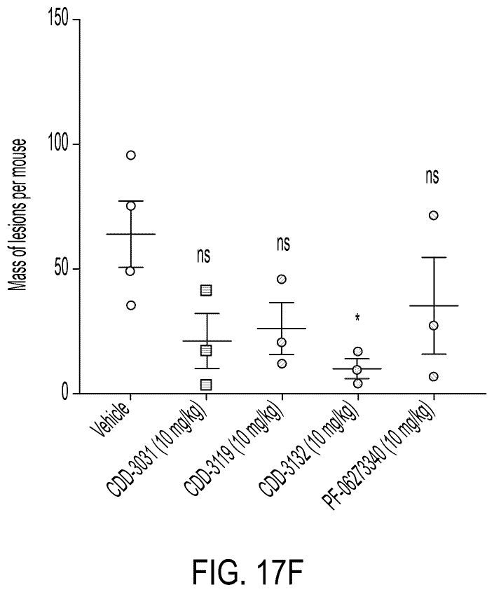

The present disclosure further demonstrates the robust efficacy of NTRK inhibitors (e.g., CDD-3132) for the inhibition of endometriotic lesion growth in mice (FIG. 17A). In certain embodiments, decreased size of endometriotic lesions was observed with administration of CDD-3132 (10 mg/kg) as compared to a vehicle control (FIGS. 17B-17C). In certain embodiments, a reduction of lesions, volume of lesions, and mass of lesions was observed with administration of CDD-3132, CDD-3119, CDD-3132, and PF-06273340 (10 mg/kg) as compared to a vehicle control (FIGS. 17D-17F).

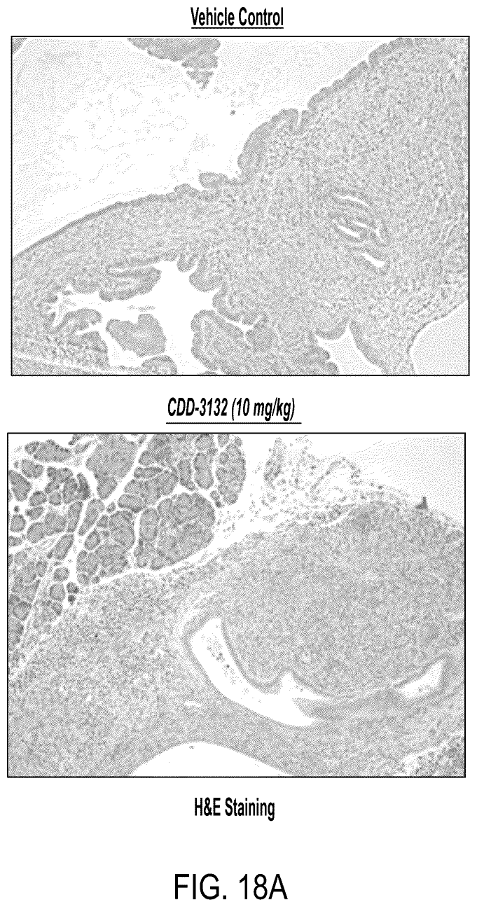

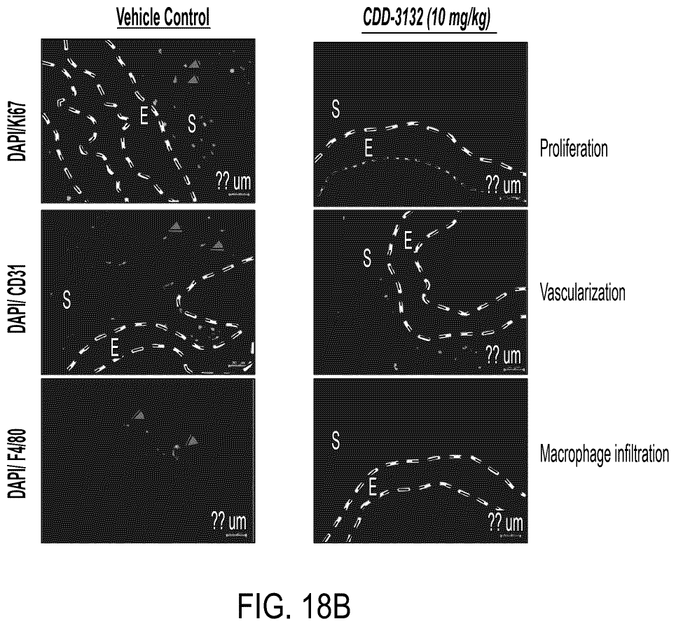

The present disclosure further demonstrates a reduction of proliferation, vascularization, macrophage infiltration, and/or inflammation within endometriotic lesions, for example, with administration of CDD-3132 (10 mg/kg) (FIGS. 18A-18B).

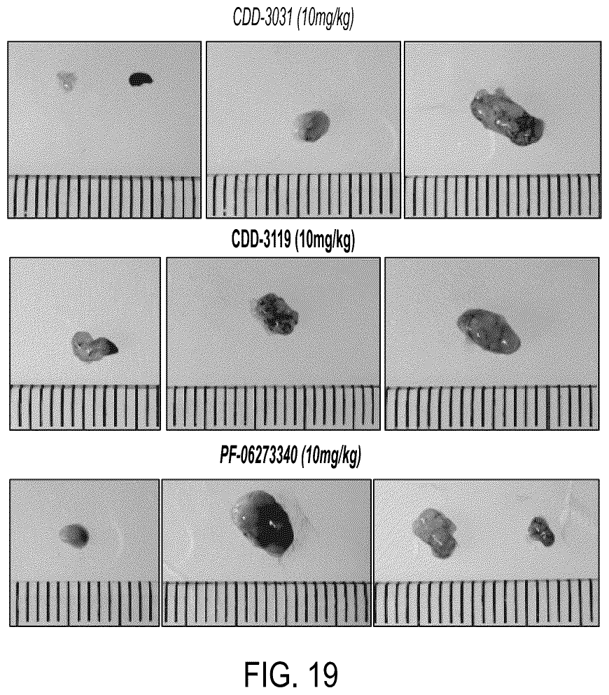

The present disclosure further demonstrates the efficacy of certain NTRK inhibitors, non-limiting examples including CDD-3031, CDD-3119, CDD-3132, and PF-06273340 (10 mg/kg), for inhibiting endometriotic lesion growth in mice (FIG. 19).

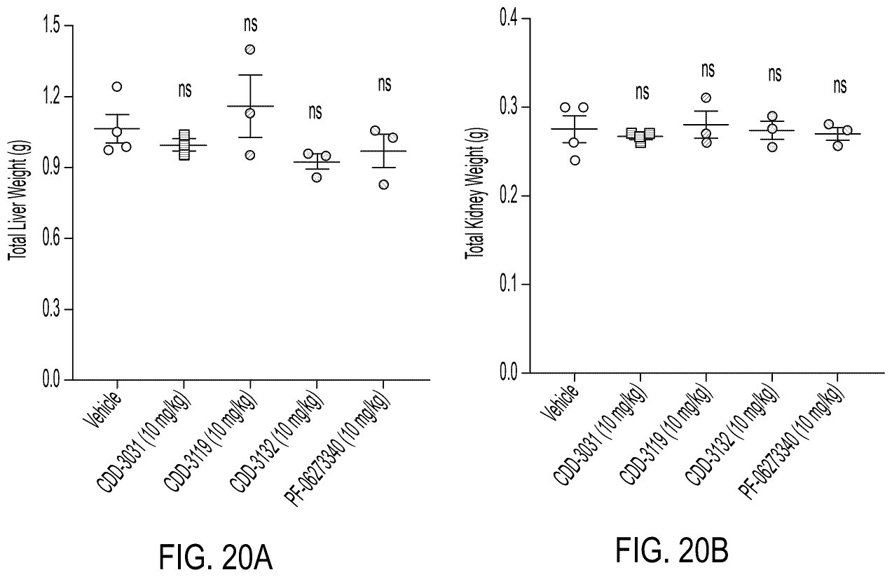

The present disclosure further demonstrates that certain NTRK2 inhibitors, non-limiting examples including CDD-3031, CDD-3119, CDD-3132, and PF-0672 (10 mg/kg) have little to no effect on liver and/or kidney weight and/or serum elevation of ALT and AST in mice with endometriotic lesions (FIGS. 20A-20B and Table 12).

TABLE 12
Evaluation of potential liver and other tissue toxicity (ALT and
AST levels) in mice with administration of exemplary NTRK inhibitors
(i.p. dose of 10 mg/kg/day for 14 days) or control (vehicle)

| Treatment | ALT (Mean ± SD) | AST (Mean ± SD) |
|---|---|---|
| Vehicle | 21.1 ± 4.2 | 51.7 ± 14.1 |
| CDD-3031 | 16.6 ± 2.6 | 46.9 ± 23.4 |
| CDD-3119 | 21.3 ± 4.9 | 53.2 ± 18.5 |
| CDD-3132 | 20.5 ± 12.6 | 33.5 ± 20.6 |
| PF-06273340 | 15.1 ± 1.6 | 25.3 ± 7.1 |

### Enumerated Embodiments

The following exemplary embodiments are provided, the numbering of which is not to be construed as designating levels of importance:

Embodiment 1 provides a compound selected from the group consisting of:
(a) a compound of formula (I):

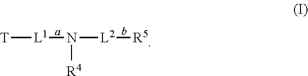

wherein:
T is selected from the group consisting of:

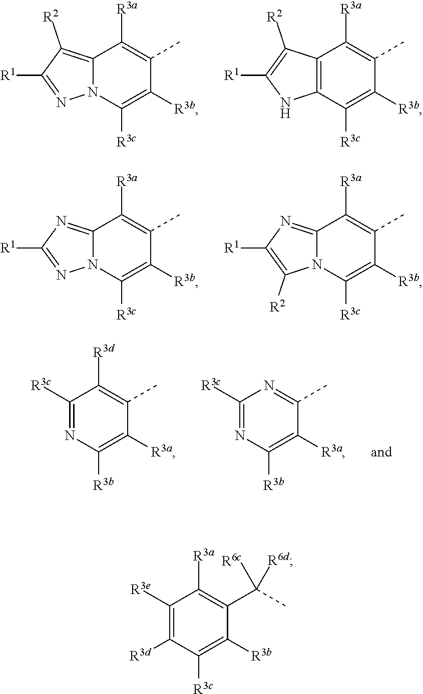

R¹ is selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R² is selected from the group consisting of H, optionally substituted C₁-C₆ alkyl, optionally substituted C₃-C₅ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}). NR^{a}C(═O)R^{b}, C(═O)N(R^{a})(OR^{b}), C(═NR^{a})N(R^{b})(R^{c}), C(═NR^{a})N(R^{b})(OR^{c}), and NR^{a}S(═O)₂R^{b};
R^{3a}, R^{3b}, R^{3c}, R^{3d}, and R^{3e} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR⁷;
R⁷ is selected from the group consisting of H and optionally substituted C₁-C₆ alkyl;
A¹ is selected from the group consisting of optionally substituted phenyl, optionally substituted naphthyl, and optionally substituted C₂-C₈ heterocyclyl;
R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl; and
wherein, if T

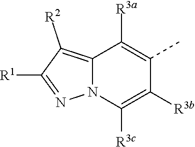

is and -L²-R⁵ is —H, then R⁴ is not H;
or a salt, solvate, isotopologue, prodrug, stereoisomer, or tautomer thereof, or any mixtures thereof;
(b) a compound of formula (II):

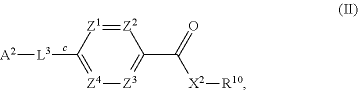

wherein:
A² is selected from the group consisting of:

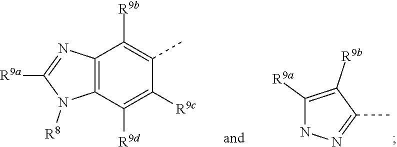

R⁸ is selected from the group consisting of H, optionally substituted C₁-C₆ alkyl, optionally substituted C₃-C₈ cycloalkyl, optionally substituted phenyl;
R^{9a}, R^{9b}, R^{9c}, and R^{9d} are each independently selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, CN, and NO₂;
R¹⁰ is selected from the group consisting of optionally substituted C₁-C₆ alkyl,

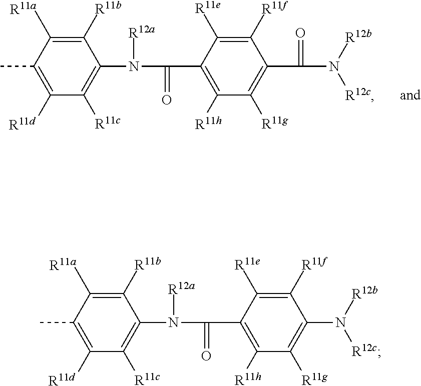

R^{11a}, R^{11b}, R^{11c}, R^{11d}, R^{11e}, R^{11f}, R^{11g} and R^{11h} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R, and NR^{a}S(═O)₂R^{b};
R^{12a}, R^{12b}, and R^{12c} are each independently selected from the group consisting of H, optionally substituted C₁-C₆ alkyl, optionally substituted C₃-C₈ cycloalkyl, optionally substituted benzyl, and optionally substituted phenyl;
R^{13a}, R^{13b}, R^{13c}, and R^{13d} are each independently selected from the group consisting of H, optionally substituted C₁-C₆ alkyl, optionally substituted C₃-C₅ cycloalkyl, optionally substituted C₁-C₆ alkoxy, halogen, CN, and NO₂;
R¹⁴ is selected from the group consisting of H and optionally substituted C₁-C₆ alkyl;
L³ is selected from the group consisting of.

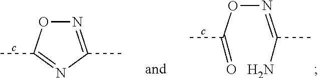

X² is selected from the group consisting of O and NR¹⁴;
Z¹ is selected from the group consisting of CR^{13a} and N, Z² is selected from the group consisting of CR^{13b} and N, Z³ is selected from the group consisting of CR^{13c} and N, and Z⁴ is selected from the group consisting of CR^{13d} and N,
   wherein at least one selected from the group consisting of Z¹, Z², Z³, and Z⁴ are N; and
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
or a salt, solvate, isotopologue, prodrug, stereoisomer, or tautomer thereof, or any mixtures thereof; and
(c) a compound of formula (III):

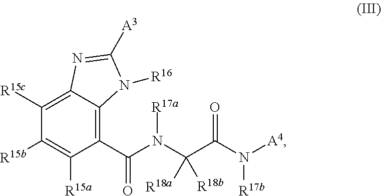

wherein:
R^{15a}, R^{15b} and R^{15c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R¹⁶ is selected from the group consisting of H, optionally substituted C₁-C₆ alkyl, optionally substituted C₃-C₅ cycloalkyl, optionally substituted benzyl, and optionally substituted phenyl;
R^{17a} and R^{17b} are each independently selected from the group consisting of H, optionally substituted C₁-C₆ alkyl, optionally substituted C₃-C₈ cycloalkyl, optionally substituted benzyl, and optionally substituted phenyl;
R^{18a} and R^{18b} are each independently selected from the group consisting of H and optionally substituted C₁-C₆ alkyl;
A³ is optionally substituted phenyl; and
A⁴ is optionally substituted phenyl; or a salt, solvate, isotopologue, prodrug, stereoisomer, or tautomer thereof, or any mixtures thereof.

Embodiment 2 provides the compound of Embodiment 1, wherein each occurrence of optionally substituted alkyl, optionally substituted cycloalkyl, optionally substituted alkoxy, optionally substituted hydroxyalkyl, optionally substituted benzyl, optionally substituted aryl, optionally substituted phenyl, optionally substituted naphthyl, optionally substituted heterocyclyl, and optionally substituted alkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₅ cycloalkyl, C₁-C₆ haloalkyl, benzyl, and phenyl.

Embodiment 3 provides the compound of Embodiment 1 or 2, which is a compound of formula (I), which is selected from the group consisting of

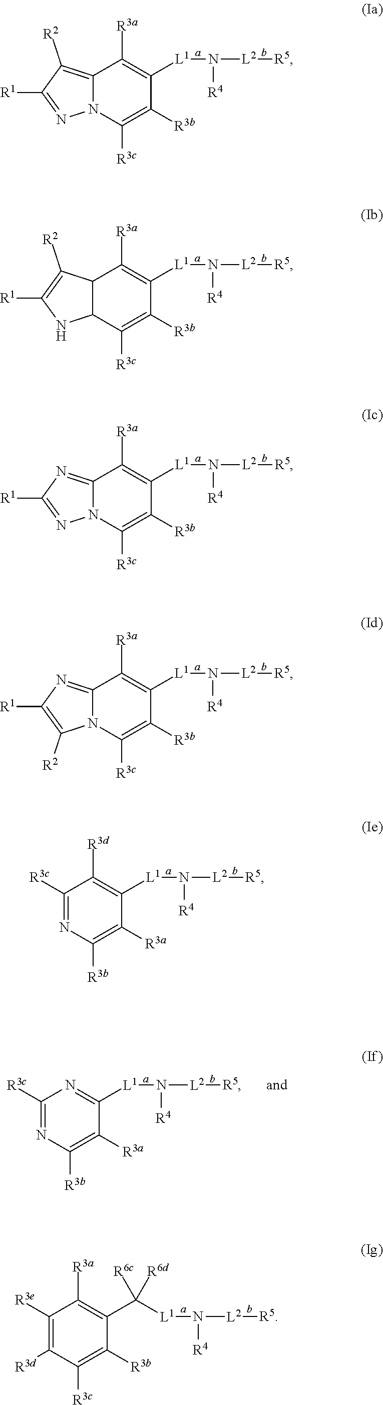

Embodiment 4 provides the compound of any one of Embodiments 1-3, wherein R¹ is H.

Embodiment 5 provides the compound of any one of Embodiments 1-4, wherein R² is selected from the group consisting of H, F, Br, CN, C(═O)OH, C(═O)NH₂, C(═O)NHMe, C(═O)NHOH, and C(═NH)NH(OH).

Embodiment 6 provides the compound of any one of Embodiments 1-5, wherein at least one of the following applies:
(a) at least one selected from R^{3a}, R^{3b}, R^{3c}, R^{3d}, and R^{3e} is H;
(b) at least two selected from R^{3a}, R^{3b}, R^{3c}, R^{3d}, and R^{3e} are H;
(c) at least three selected from R^{3a}, R^{3b}, R³¹, R^{3d}, and R^{3e} are H;
(d) at least four selected from R^{3a}, R^{3b}, R^{3c}, R^{3d}, and R^{3e} are H; and
(e) each of R^{3a}, R^{3b}, R^{3c}, R^{3d}, and R^{3e} are H.

Embodiment 7 provides the compound of any one of Embodiments 1-6, wherein T is selected from the group consisting of

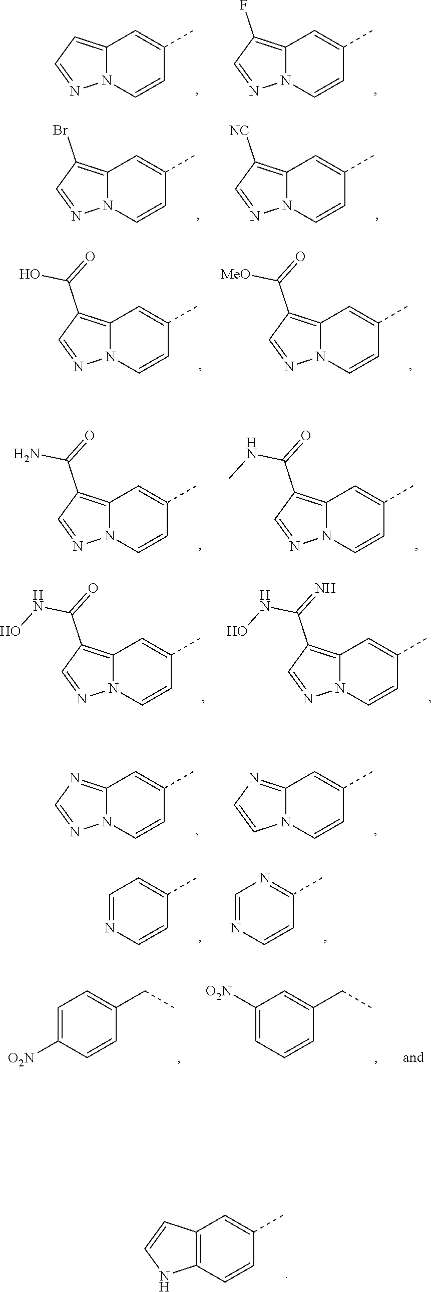

Embodiment 8 provides the compound of any one of Embodiments 1-7, wherein R⁴ is selected from the group consisting of H, methyl, isopropyl, 1-methylpropyl, 2-methylpropyl. 3-methylbutyl, cyclopropyl, cyclobutyl, cyclopentyl, cyclohexyl, cycloheptyl, (CH₂)₃N(CH₃)₂, C(═O)CH₃, phenyl, benzyl substituted with at least one C₁-C₆ alkyl; and —CH₂CH₂CH₂O(phenyl substituted with at least one halogen).

Embodiment 9 provides the compound of any one of Embodiments 1-7, wherein R^{a} is selected from the group consisting of

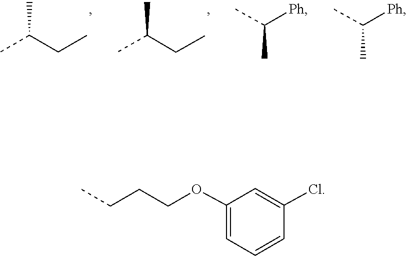

Embodiment 10 provides the compound of any one of Embodiments 1-9, wherein A¹ is selected from the group consisting of H; phenyl substituted with at least one substituent selected from the group consisting of C₁-C₆ alkyl, C₁-C₆ alkoxy, C₁-C₃ haloalkyl, C₁-C₃ haloalkoxy, C₃-C₈ cycloalkyl optionally substituted with a C₁-C₃ haloalkyl, phenoxy, halogen, and NO₂; phenyl fused with a C₂-C₆ heterocycloalkyl; and C₂-C₈ heteroaryl optionally substituted with C₁-C₆ alkyl or fused with a C₂-C₅ heteroaryl.

Embodiment 11 provides the compound of any one of Embodiments 1-10, wherein A¹ is selected from the group consisting of

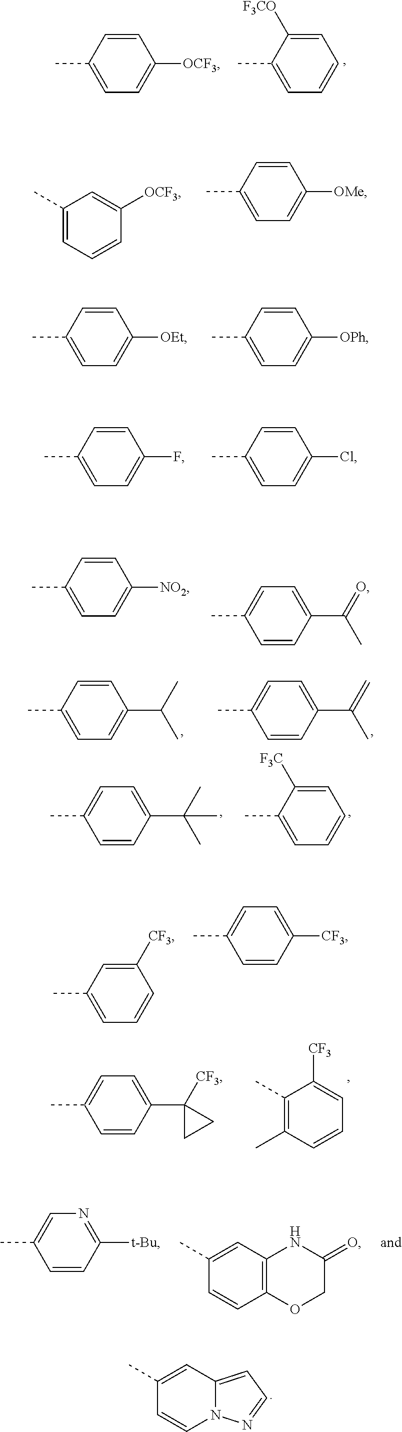

Embodiment 12 provides the compound of any one of Embodiments 1-11, wherein L¹ is —CH₂—.

Embodiment 13 provides the compound of any one of Embodiments 1-12, wherein L² is selected from the group consisting of a bond, —C(═O)—, —C(═S)—, S(═O)₂-,

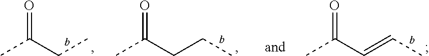

Embodiment 14 provides the compound of any one of Embodiments 1-13, wherein X¹ is selected from the group consisting of a bond, O, and NR⁷.

Embodiment 15 provides the compound of Embodiment 14, wherein R⁷ is selected from the group consisting of H and Me.

Embodiment 16 provides the compound of any one of Embodiments 1-15, wherein R⁵ is selected from the group consisting of

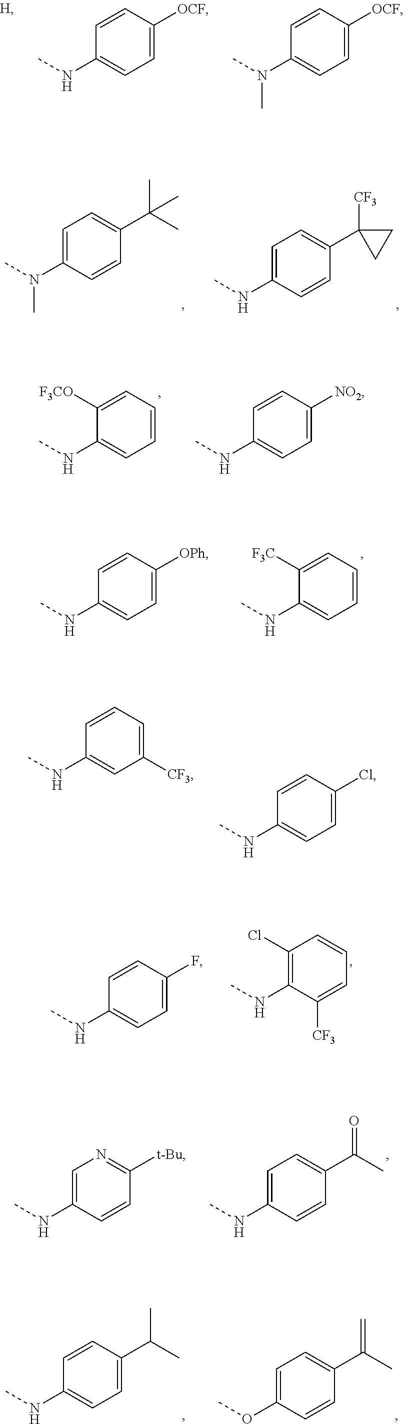

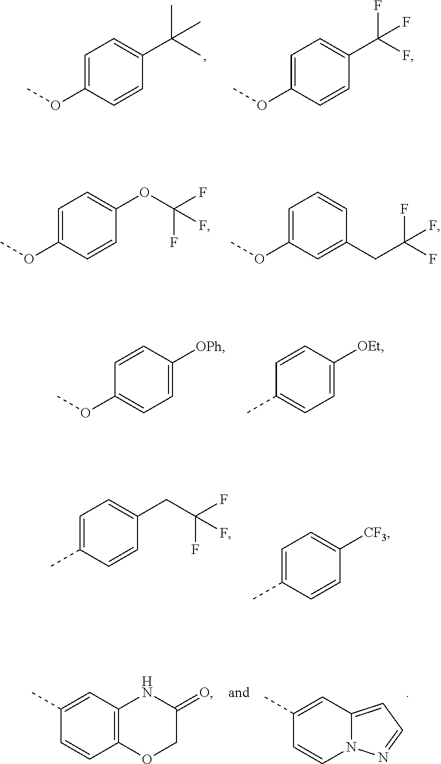

Embodiment 17 provides the compound of any one of Embodiments 1-16, which is selected from the group consisting of:
5-((1-isopropyl-3-(4-(trifluoromethoxy)phenyl)ureido)methyl)pyrazolo[1,5-a]pyridine-3-carboxylic acid;
1-cycloheptyl-1-(pyrazolo[1,5-a]pyridin-5-ylmethyl)-3-(4-(trifluoromethoxy)phenyl)urea;
1-cyclobutyl-1-(pyrazolo[1,5-a]pyridin-5-ylmethyl)-3-(2-(trifluoromethoxy)phenyl)urea;
1-isopropyl-1-(pyrazolo[1,5-a]pyridin-5-ylmethyl)-3-(4-(trifluoromethoxy)phenyl)urea;
1-cyclohexyl-1-(pyrazolo[1,5-a]pyridin-5-ylmethyl)-3-(4-(trifluoromethoxy)phenyl)urea;
1-cyclobutyl-3-(4-nitrophenyl)-1-(pyrazolo[1,5-a]pyridin-5-ylmethyl)urea;
1-phenyl-1-(pyrazolo[1,5-a]pyridin-5-ylmethyl)-3-(4-(trifluoromethoxy)phenyl)urea;
1-cyclopentyl-1-(pyrazolo[1,5-a]pyridin-5-ylmethyl)-3-(4-(trifluoromethoxy)phenyl)urea;
1-cyclopropyl-1-(pyrazolo[1,5-a]pyridin-5-ylmethyl)-3-(4-(trifluoromethoxy)phenyl)urea;
1-cyclobutyl-1-(pyrazolo[1,5-a]pyridin-5-ylmethyl)-3-(2-(trifluoromethyl)phenyl)urea;
1-cyclobutyl-1-(pyrazolo[1,5-a]pyridin-5-ylmethyl)-3-(3-(trifluoromethyl)phenyl)urea;
N-(3-(3-chlorophenoxy)propyl)-2-(4-isopropylphenoxy)-N-(pyrazolo[1,5-a]pyridin-5-ylmethyl)acetamide;
methyl 5-((cyclobutylamino)methyl)pyrazolo[1,5-a]pyridine-3-carboxylate;
3-(3-chlorophenoxy)-N-(pyrazolo[1,5-a]pyridin-5-ylmethyl)propan-1-amine;
N,N-bis(pyrazolo[1,5-a]pyridin-5-ylmethyl)cyclobutanamine;
1-(3-(3-chlorophenoxy)propyl)-3-(4-phenoxyphenyl)-1-(pyrazolo[1,5-a]pyridin-5-ylmethyl)urea;
1-(3-(3-chlorophenoxy)propyl)-1-(pyrazolo[1,5-a]pyridin-5-ylmethyl)-3-(4-(trifluoromethoxy)phenyl)urea;
3-(4-chlorophenyl)-1-cyclobutyl-1-(pyrazolo[1,5-a]pyridin-5-ylmethyl)urea;
1-cyclobutyl-3-(4-fluorophenyl)-1-(pyrazolo[1,5-a]pyridin-5-ylmethyl)urea;
5-((1-cyclobutyl-3-(4-(trifluoromethoxy)phenyl)ureido)methyl)-N-methylpyrazolo[1,5-a]pyridine-3-carboxamide;
5-((1-cyclobutyl-3-(4-(trifluoromethoxy)phenyl)ureido)methyl)pyrazolo[1,5-a]pyridine-3-carboxylic acid;
methyl 5-((1-cyclobutyl-3-(4-(trifluoromethoxy)phenyl)ureido)methyl)pyrazolo[1,5-a]pyridine-3-carboxylate;
1-cyclobutyl-3-(4-methoxyphenyl)-1-(pyrazolo[1,5-a]pyridin-5-ylmethyl)urea;
(R)-2-(4-ethoxyphenyl)-N-(1-phenylethyl)-N-(pyrazolo[1,5-a]pyridin-5-ylmethyl)acetamide;
(S)-2-(4-ethoxyphenyl)-N-(1-phenylethyl)-N-(pyrazolo[1,5-a]pyridin-5-ylmethyl)acetamide;
1-cyclobutyl-1-(pyrazolo[1,5-a]pyridin-5-ylmethyl)-3-(4-(trifluoromethoxy)phenyl)urea;
(S)-1-phenyl-N-(pyrazolo[1,5-a]pyridin-5-ylmethyl)ethan-1-amine;
1-((3-fluoropyrazolo[1,5-a]pyridin-5-yl)methyl)-1-isopropyl-3-(4-(trifluoromethoxy)phenyl)urea;
1-((3-bromopyrazolo[1,5-a]pyridin-5-yl)methyl)-1-isopropyl-3-(4-(trifluoromethoxy)phenyl)urea;
5-((1-cyclopropyl-3-(4-(trifluoro methoxy)phenyl) ureido)methyl)pyrazolo[1,5-a]pyridine-3-carboxylic acid;
5-((1-isopropyl-3-(4-(trifluoro methoxy)phenyl) ureido)methyl)pyrazolo[1,5-a]pyridine-3-carboxamide;
5-((1-cyclopropyl-3-(4-(trifluoro methoxy)phenyl) ureido)methyl)pyrazolo[1,5-a]pyridine-3-carboxamide;
5-((1-isopropyl-3-methyl-3-(4-(trifluoro methoxy) phenyl)ureido)methyl)pyrazolo[1,5-a]pyridine-3-carboxylic acid;
N-hydroxy-5-((1-isopropyl-3-methyl-3-(4-(trifluoro methoxy)phenyl)ureido)methyl)pyrazolo[1,5-a]pyridine-3-carboxamide;
1-((3-cyanopyrazolo[1,5-a]pyridin-5-yl)methyl)-1-isopropyl-3-(4-(trifluoromethoxy)phenyl)urea;
5-((1-isobutyl-3-(4-(trifluoromethoxy)phenyl)ureido)methyl)pyrazolo[1,5-a]pyridine-3-carboxylic acid;
5-((1-(sec-butyl)-3-(4-(trifluoromethoxy)phenyl) ureido)methyl)pyrazolo[1,5-a]pyridine-3-carboxylic acid;
N-hydroxy-5-((1-isopropyl-3-(4-(trifluoromethoxy) phenyl)ureido)methyl)pyrazolo[1,5-a]pyridine-3-carboximidamide;
5-((1-methyl-3-(4-(trifluoromethoxy)phenyl)thioureido)methyl)pyrazolo[1,5-a]pyridine-3-carboxamide;
5-((1-isopropyl-3-(4-(trifluoromethoxy)phenyl)thioureido)methyl)pyrazolo[1,5-a]pyridine-3-carboxamide;
5-((2-(4-isopropylphenoxy)-N-methylacetamido)methyl)pyrazolo[1,5-a]pyridine-3-carboxamide;
5-((N-methyl-3-(4-(trifluoromethoxy)phenyl)propanamido)methyl)pyrazolo[1,5-a]pyridine-3-carboxamide;
5-((1-methyl-3-(4-(trifluoromethoxy)phenyl)ureido)methyl)pyrazolo[1,5-a]pyridine-3-carboxamide;
5-((N-isopropyl-2-(4-isopropylphenoxy)acetamido)methyl)pyrazolo[1,5-a]pyridine-3-carboxamide;
5-((N-(3-(dimethylamino)propyl)-2-(4-isopropylphenoxy)acetamido)methyl)pyrazolo[1,5-a]pyridine-3-carboxamide;
5-((2-(4-isopropylphenoxy)acetamido)methyl)pyrazolo[1,5-a]pyridine-3-carboxamide;
5-((3-(4-(trifluoromethoxy)phenyl)ureido)methyl)pyrazolo[1,5-a]pyridine-3-carboxamide;
5-((3-(4-(trifluoromethoxy)phenyl)thioureido)methyl)pyrazolo[1,5-a]pyridine-3-carboxamide;
5-((N-methyl-2-(4-(trifluoromethoxy)phenoxy)acetamido)methyl)pyrazolo[1,5-a]pyridine-3-carboxamide;
5-((N-isopropyl-2-(4-(trifluoromethoxy)phenoxy)acetamido)methyl)pyrazolo[1,5-a]pyridine-3-carboxamide;
5-((1-(sec-butyl)-3-(4-(trifluoromethoxy)phenyl)ureido)methyl)pyrazolo[1,5-a]pyridine-3-carboxamide;
(R)-5-((1-(sec-butyl)-3-(4-(trifluoromethoxy)phenyl)ureido)methyl)pyrazolo[1,5-a]pyridine-3-carboxamide;
(S)-5-((1-(sec-butyl)-3-(4-(trifluoromethoxy)phenyl)ureido)methyl)pyrazolo[1,5-a]pyridine-3-carboxamide;
5-((N-methyl-2-(4-(trifluoromethyl)phenoxy)acetamido)methyl)pyrazolo[1,5-a]pyridine-3-carboxamide;
5-((N-methyl-2-(3-(trifluoromethoxy)phenoxy)acetamido)methyl)pyrazolo[1,5-a]pyridine-3-carboxamide;
5-((2-(4-(tert-butyl)phenoxy)-N-methylacetamido)methyl)pyrazolo[1,5-a]pyridine-3-carboxamide;
5-((2-(4-(tert-butyl)phenoxy)-N-isopropylacetamido)methyl)pyrazolo[1,5-a]pyridine-3-carboxamide;
5-((2-((4-(tert-butyl)phenyl)(methyl)amino)-N-methylacetamido)methyl)pyrazolo[1,5-a]pyridine-3-carboxamide;
5-((N-isopropyl-2-((4-(trifluoromethoxy)phenyl)amino)acetamido)methyl)pyrazolo[1,5-a]pyridine-3-carboxamide;
5-((N-isopropyl-2-(4-(prop-1-en-2-yl)phenoxy)acetamido)methyl)pyrazolo[1,5-a]pyridine-3-carboxamide;
5-((2-(4-acetylphenoxy)-N-isopropylacetamido)methyl)pyrazolo[1,5-a]pyridine-3-carboxamide;
5-((2-(4-acetylphenoxy)-N-methylacetamido)methyl)pyrazolo[1,5-a]pyridine-3-carboxamide;
5-((N-methyl-2-((4-(trifluoromethoxy)phenyl)amino)acetamido)methyl)pyrazolo[1,5-a]pyridine-3-carboxamide;
2-(3-(2-chloro-6-(trifluoromethyl)phenyl)ureido)-N,N-dimethyl-3-(3-nitrophenyl)propenamide;
(S)-2-(3-(2-chloro-6-(trifluoromethyl)phenyl)ureido)-N,N-dimethyl-3-(3-nitrophenyl)propenamide;
(R)-2-(3-(2-chloro-6-(trifluoromethyl)phenyl)ureido)-N,N-dimethyl-3-(3-nitrophenyl)propenamide;
N-methyl-3-(4-nitrophenyl)-2-(3-(4-phenoxyphenyl)ureido)propenamide;
(R)-N-methyl-3-(4-nitrophenyl)-2-(3-(4-phenoxyphenyl)ureido)propenamide;
(S)-N-methyl-3-(4-nitrophenyl)-2-(3-(4-phenoxyphenyl)ureido)propenamide;
N-((1H-indol-5-yl)methyl)-N-isopropyl-2-(4-(trifluoromethoxy)phenoxy)acetamide;
1-((1H-indol-5-yl)methyl)-1-isopropyl-3-(4-(trifluoromethoxy)phenyl)urea;
2-((6-(tert-butyl)pyridin-3-yl)amino)-N-isopropyl-N-(pyrazolo[1,5-a]pyridin-5-ylmethyl)acetamide;
N-isopropyl-N-(pyrazolo[1,5-a]pyridin-5-ylmethyl)-2-((4-(1-(trifluoromethyl)cyclopropyl)phenyl)amino)acetamide;
5-((methyl(N-(4-(trifluoromethoxy)phenyl)sulfamoyl)amino)methyl)pyrazolo[1,5-a]pyridine-3-carboxamide;
1-([1,2,4]triazolo[1,5-a]pyridin-7-ylmethyl)-1-cyclopropyl-3-(4-(trifluoromethoxy)phenyl)urea;
1-cyclopropyl-1-(pyridin-4-ylmethyl)-3-(4-(trifluoromethoxy)phenyl)urea;
1-cyclopropyl-1-(pyrimidin-4-ylmethyl)-3-(4-(trifluoromethoxy)phenyl)urea;
1-cyclopropyl-1-(imidazo[1,2-a]pyridin-7-ylmethyl)-3-(4-(trifluoromethoxy)phenyl)urea;
5-((N-(3-(3-chlorophenoxy)propyl)-2-(4-isopropylphenoxy)acetamido)methyl)pyrazolo[1,5-a]pyridine-3-carboxamide;
5-((N-(4-(trifluoromethoxy)phenyl)acetamido)methyl)pyrazolo[1,5-a]pyridine-3-carboxamide;
5-(((N-isopropyl-4-(trifluoromethyl)phenyl)sulfonamido)methyl)pyrazolo[1,5-a]pyridine-3-carboxamide;
5-(((N-isopropyl-3-oxo-3,4-dihydro-2H-benzo[b][1,4]oxazine)-6-sulfonamido)methyl)pyrazolo[1,5-a]pyridine-3-carboxamide;
5-(((N-methyl-4-(trifluoromethoxy)phenyl)sulfonamido)methyl)pyrazolo[1,5-a]pyridine-3-carboxamide;
5-(((N-isopropyl-4-(trifluoromethoxy)phenyl)sulfonamido)methyl)pyrazolo[1,5-a]pyridine-3-carboxamide;
5-((N-isopropyl-3-(4-methoxyphenyl)acrylamido)methyl)pyrazolo[1,5-a]pyridine-3-carboxamide;
(E)-5-((N-isopropyl-3-(4-methoxyphenyl)acrylamido)methyl)pyrazolo[1,5-a]pyridine-3-carboxamide;
(Z)-5-((N-isopropyl-3-(4-methoxyphenyl)acrylamido)methyl)pyrazolo[1,5-a]pyridine-3-carboxamide;
5-((N-isopropyl-3-(4-(trifluoromethyl)phenyl)acrylamido)methyl)pyrazolo[1,5-a]pyridine-3-carboxamide;
(E)-5-((N-isopropyl-3-(4-(trifluoromethyl)phenyl)acrylamido)methyl)pyrazolo[1,5-a]pyridine-3-carboxamide; and
(Z)-5-((N-isopropyl-3-(4-(trifluoromethyl)phenyl)acrylamido)methyl)pyrazolo[1,5-a]pyridine-3-carboxamide.

Embodiment 18 provides the compound of Embodiment 1 or 2, which is a compound of formula (II).

Embodiment 19 provides the compound of Embodiment 18, wherein R⁸ is selected from the group consisting of isopropyl and phenyl.

Embodiment 20 provides the compound of Embodiment 18 or 19, wherein R^{9a}, R^{9b}, R^{9C}, and R^{9d} are each independently selected from the group consisting of H, tert-butyl, and trifluoromethyl.

Embodiment 21 provides the compound of any one of Embodiments 18-20, wherein A² is selected from the group consisting of

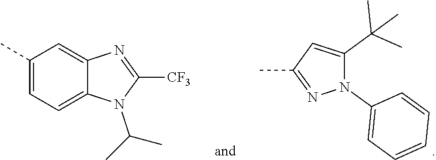

Embodiment 22 provides the compound of any one of Embodiments 17-20, wherein one of the following applies:
(a) Z¹ is N and at least one of Z², Z³, and Z⁴ is CH;
(b) Z¹ is N and at least two of Z², Z³, and Z⁴ are CH;
(c) Z¹ is N and each of Z², Z³, and Z⁴ are CH;
(d) Z² is N and at least one of Z¹, Z³, and Z⁴ is CH;
(e) Z² is N and at least two of Z¹, Z³, and Z⁴ are CH; or
(f) Z² is N and each of Z¹, Z³, and Z⁴ are CH.

Embodiment 23 provides the compound of any one of Embodiments 18-22, wherein X² is selected from the group consisting of —O— and —NH—.

Embodiment 24 provides the compound of any one of Embodiments 18-23, wherein R^{11a}, R^{11b}, R^{11c}, R^{11d}, R^{11e}, R^{11f}, R^{11g}, and R^{11h} are each independently selected from the group consisting of H and methoxy.

Embodiment 25 provides the compound of any one of Embodiments 18-24, wherein R^{12a}, R^{12b}, and R^{12c} are each independently selected from the group consisting of H, methyl, and C(═O)O(C(CH₃)₃).

Embodiment 26 provides the compound of any one of Embodiments 18-25, wherein R¹⁰ is selected from the group consisting of tert-butyl,

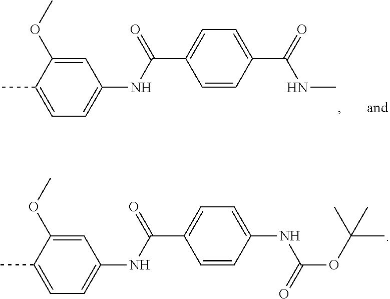

Embodiment 27 provides the compound of any one of Embodiments 1-2 and 18-26, which is selected from the group consisting of:
N¹-(4-(5-(5-(5-(tert-butyl)-1-phenyl-1H-pyrazol-3-yl)-1,2,4-oxadiazol-3-yl)picolinamido)-3-methoxyphenyl)-N⁴-methylterephthalamide;
N¹-(4-(5-(5-(1-isopropyl-2-(trifluoromethyl)-1H-benzo[d]imidazol-5-yl)-1,2,4-oxadiazol-3-yl)picolinamido)-3-methoxyphenyl)-N⁴-methylterephthalamide;
tert-butyl 5-(5-(5-(tert-butyl)-1-phenyl-1H-pyrazol-3-yl)-1,2,4-oxadiazol-3-yl)picolinate;
tert-butyl 5-(5-(1-isopropyl-2-(trifluoromethyl)-1H-benzo[d]imidazol-5-yl)-1,2,4-oxadiazol-3-yl)picolinate;
(Z)-N¹-(4-(5-(N′-((5-(tert-butyl)-1-phenyl-1H-pyrazole-3-carbonyl)oxy)carbamimidoyl)picolinamido)-3-methoxyphenyl)-N⁴-methylterephthalamide; and
tert-butyl (4-(5-(5-(1-isopropyl-2-(trifluoromethyl)-1H-benzo[d]imidazol-5-yl)-1,2,4-oxadiazol-3-yl)picolinamido)-3-methoxyphenyl)carbamate.

Embodiment 28 provides the compound of Embodiment 1 or 2, wherein the compound is a compound of formula (III).

Embodiment 29 provides the compound of Embodiment 28, wherein the compound of formula (III) is selected from the group consisting of:

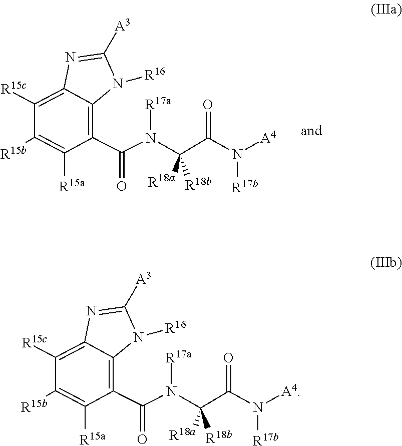

Embodiment 30 provides the compound of Embodiment 28 or 29, wherein at least one of the following applies:
(a) at least one of R^{15a}, R^{15b}, and R^{15c} is H;
(b) at least two of R^{15a}, R^{15b}, and R^{15c} are H; and
(c) each of R^{15a}, R^{15b}, and R^{15c} are H.

Embodiment 31 provides the compound of any one of Embodiments 28-30, wherein R¹⁶ is selected from the group consisting of benzyl and

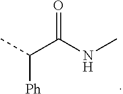

Embodiment 32 provides the compound of any one of Embodiments 28-31, wherein R^{17a} and R^{17b} are each independently H.

Embodiment 33 provides the compound of any one of Embodiments 28-32, wherein R^{18a} and R^{18b} are each independently selected from the group consisting of H and Me.

Embodiment 34 provides the compound of any one of Embodiments 28-33, wherein A³ is selected from the group consisting of

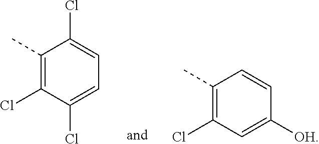

Embodiment 35 provides the compound of any one of Embodiments 28-34, wherein A⁴ is

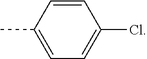

Embodiment 36 provides the compound of any one of Embodiments 1-2 and 28-35, which is selected from the group consisting of
N-((S)-1-((4-chlorophenyl)amino)-1-oxopropan-2-yl)-1-(2-(methylamino)-2-oxo-1-phenylethyl)-2-(2,3,6-trichlorophenyl)-1H-benzo[d]imidazole-7-carboxamide;
(S)-1-benzyl-2-(2-chloro-4-hydroxyphenyl)-N-(1-((4-chlorophenyl)amino)-1-oxopropan-2-yl)-1H-benzo[d]imidazole-7-carboxamide; and
(S)-1-benzyl-N-(1-((4-chlorophenyl)amino)-1-oxopropan-2-yl)-2-(2,3,6-trichlorophenyl)-1H-benzo[d]imidazole-7-carboxamide.

Embodiment 37 provides the pharmaceutical composition comprising the compound of any one of Embodiments 1-36 and a pharmaceutically acceptable carrier.

Embodiment 38 provides a method of treating, preventing, and/or ameliorating endometriosis in a subject in need thereof, the method comprising administering to the subject at least one Neurotrophic Receptor Tyrosine Kinase (NTRK) inhibitor or a pharmaceutical composition comprising the NTRK inhibitor and a pharmaceutically acceptable carrier.

Embodiment 39 provides the method of Embodiment 38, wherein at least one selected from the group consisting of chronic pelvic pain, inflammation, and infertility is treated, prevented, and/or ameliorated in the subject.

Embodiment 40 provides the method of Embodiment 38 or 39, wherein the subject is a female.

Embodiment 41 provides a method of treating, preventing, and/or ameliorating cancer in a subject in need thereof, the method comprising administering to the subject at least one Neurotrophic Receptor Tyrosine Kinase (NTRK) inhibitor or a pharmaceutical composition comprising the NTRK inhibitor and a pharmaceutically acceptable carrier.

Embodiment 42 provides a method of treating, preventing, and/or ameliorating pain in a subject in need thereof, the method comprising administering to the subject at least one Neurotrophic Receptor Tyrosine Kinase (NTRK) inhibitor or a pharmaceutical composition comprising the NTRK inhibitor and a pharmaceutically acceptable carrier.

Embodiment 43 provides the method of any one of Embodiments 38-42, wherein at least one selected from the group consisting of neurotrophic receptor tyrosine kinase 1 (NTRK1), neurotrophic receptor tyrosine kinase 2 (NTRK2), and neurotrophic receptor tyrosine kinase 3 (NTRK3), is overexpressed in the subject.

Embodiment 44 provides the method of any one of Embodiments 38-44, wherein at least one selected from the group consisting of NTRK1, NTRK2, and NTRK3 is inhibited in the subject.

Embodiment 45 provides the method of any one of Embodiments 38-44, wherein a fusion gene comprising at least one selected from the group consisting of NTRK1, NTRK2, and NTRK3 is overexpressed in the subject.

Embodiment 46 provides the method of Embodiment 45, wherein the fusion gene product is inhibited in the subject.

Embodiment 47 provides the method of any one of Embodiments 38-46, wherein the NTRK inhibitor comprises the compound of any one of Embodiments 1-35 or the pharmaceutical composition thereof comprises the pharmaceutical composition of Embodiment 36.

Embodiment 48 provides the method of any one of Embodiments 38-47, wherein the NTRK inhibitor is a NTRK2 inhibitor.

Embodiment 49 provides the method of Embodiment 48, wherein the NTRK2 inhibitor selectively inhibits NTRK2 over NTRK1 and/or NTRK3.

Embodiment 50 provides the method of any one of Embodiments 38-49, wherein the at least one NTRK2 inhibitor is selected from the group consisting of staurosporine, larotrectinib, entrectinib, selitrectinib, reprotrectinib, and PF-06273340.

Embodiment 51 provides the method of any one of Embodiments 38-50, wherein the subject is a mammal.

Embodiment 52 provides the method of Embodiment 51, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound selected from the group consisting of:
(a) a compound of formula (I): 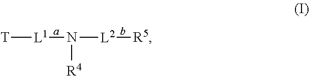
wherein:
T is selected from the group consisting of: 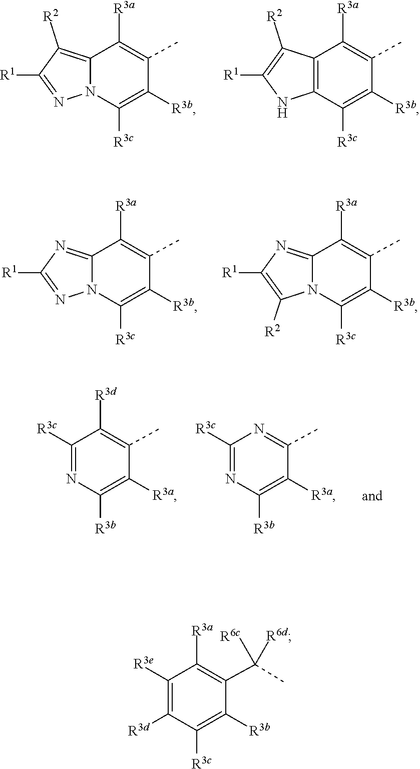
R¹ is selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, C(═O)OR^{a}, C(═O)N(R^{a})(R^{b}), S(═O)₂N(R^{a})(R^{b}), CN, and NO₂;
R² is selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{b}, C(═O)N(R^{a})(OR^{b}), C(═NR^{a})N(R^{b})(R^{c}), C(═NR^{a})N(R^{b})(OR^{c}), and NR^{a}S(═O)₂R^{b};
R^{3a}, R^{3b}, R^{3c}, R^{3d}, and R^{3e} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R⁴ is selected from the group consisting of H, optionally substituted C₁-C₆ alkyl, C(═O)(optionally substituted C₁-C₆ alkyl), optionally substituted C₃-C₈ cycloalkyl, optionally substituted C₁-C₆ aminoalkyl, optionally substituted C₂-C₆ hydroxyalkyl, optionally substituted benzyl, and optionally substituted phenyl;
R⁵ is selected from the group consisting of H and —X¹-A¹;
R^{6a}, R^{6b}, R^{6c}, and R^{6d} are each independently selected from the group consisting of H, optionally substituted C₁-C₆ alkyl, and C(═O)N(R^{a})(R^{b});
L¹ is —C(R^{6a})(R^{6b})—;
L² is selected from the group consisting of a bond, —C(═O)—, —C(═O)(optionally substituted C₁-C₃ alkylenyl), —C(═O)(optionally substituted C₂-C₃ alkenylenyl), —C(═S)—, —C(═S)(optionally substituted C₁-C₃ alkylenyl), —C(═S)(optionally substituted C₂-C₃ alkenylenyl), —S(═O)₂—, —S(═O)₂(optionally substituted C₁-C₃ alkylenyl), —S(═O)₂(optionally substituted C₂-C₃ alkenylenyl), optionally substituted C₁-C₃ alkylenyl, and optionally substituted C₂-C₃ alkenylenyl;
X¹ is selected from the group consisting of a bond, O, and NR₇;
R⁷ is selected from the group consisting of H and optionally substituted C₁-C₆ alkyl;
A¹ is selected from the group consisting of optionally substituted phenyl, optionally substituted naphthyl, and optionally substituted C₂-C₈ heterocyclyl;
R^{a}, R^{b}, and R^{c} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl; and
wherein, if 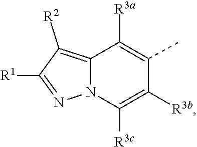
is and -L²-R⁵ is —H, then R⁴ is not H;
or a salt, solvate, isotopologue, prodrug, stereoisomer, or tautomer thereof, or any mixtures thereof;
(b) a compound of formula (II): 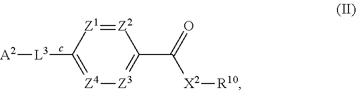
wherein:
A² is selected from the group consisting of: 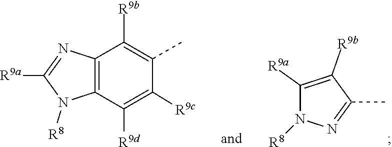
R⁸ is selected from the group consisting of H, optionally substituted C₁-C₆ alkyl, optionally substituted C₃-C₈ cycloalkyl, optionally substituted phenyl;
R^{9a}, R^{9b}, R^{9c}, and R^{9d} are each independently selected from the group consisting of H, optionally substituted C₁-C₆ alkyl, optionally substituted C₃-C₈ cycloalkyl, optionally substituted C₁-C₆ alkoxy, optionally substituted C₆-C₁₀ aryl, optionally substituted C₂-C₈ heterocyclyl, halogen, CN, and NO₂;
R¹⁰ is selected from the group consisting of optionally substituted C₁-C₆ alkyl, 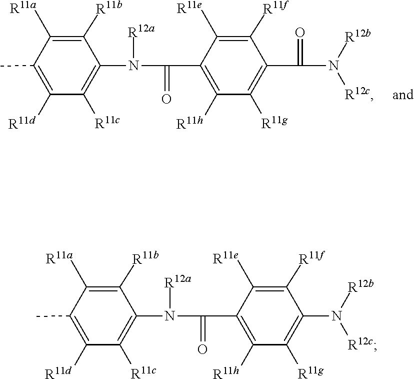
R^{11a}, R^{11b}, R^{11c}, R^{11d}, R^{11e}, R^{11f}, R^{11g} and R^{11h} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{b}, and NR^{a}S(═O)₂R^{b};
R^{12a}, R^{12b}, and R^{12c} are each independently selected from the group consisting of H, optionally substituted C₁-C₆ alkyl, optionally substituted C₃-C₈ cycloalkyl, optionally substituted benzyl, and optionally substituted phenyl;
R^{13a}, R^{13b}, R^{13c}, and R^{13d} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R¹⁴ is selected from the group consisting of H and optionally substituted C₁-C₆ alkyl;
L³ is selected from the group consisting of: 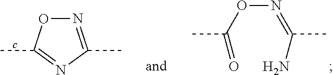
X² is selected from the group consisting of O and NR¹⁴;
Z¹ is selected from the group consisting of CR^{13a} and N, Z² is selected from the group consisting of CR^{13b} and N, Z³ is selected from the group consisting of CR^{13c} and N, and Z⁴ is selected from the group consisting of CR^{13d} and N,
wherein at least one selected from the group consisting of Z¹, Z², Z³, and Z⁴ are N; and
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
or a salt, solvate, isotopologue, prodrug, stereoisomer, or tautomer thereof, or any mixtures thereof, and
(c) a compound of formula (III): 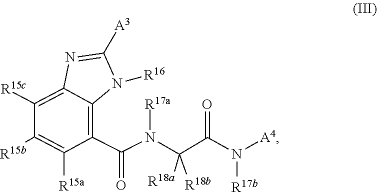
wherein:
R^{15a}, R^{15b}, and R^{15c} are each independently selected from the group consisting of H, optionally substituted C₁-C₆ alkyl, optionally substituted C₃-C₈ cycloalkyl, optionally substituted C₁-C₆ alkoxy, halogen, CN, and NO₂;
R¹⁶ is selected from the group consisting of H, optionally substituted C₁-C₆ alkyl, optionally substituted C₃-C₈ cycloalkyl, optionally substituted benzyl, and optionally substituted phenyl;
R^{17a} and R^{17b} are each independently selected from the group consisting of H, optionally substituted C₁-C₆ alkyl, optionally substituted C₃-C₈ cycloalkyl, optionally substituted benzyl, and optionally substituted phenyl;
R^{18a} and R^{18b} are each independently selected from the group consisting of H and optionally substituted C₁-C₆ alkyl;
A³ is optionally substituted phenyl; and
A⁴ is optionally substituted phenyl;
or a salt, solvate, isotopologue, prodrug, stereoisomer, or tautomer thereof, or any mixtures thereof.

2. The compound of claim 1, wherein each occurrence of optionally substituted alkyl, optionally substituted cycloalkyl, optionally substituted alkoxy, optionally substituted hydroxyalkyl, optionally substituted benzyl, optionally substituted aryl, optionally substituted phenyl, optionally substituted naphthyl, optionally substituted heterocyclyl, and optionally substituted alkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

3. The compound of claim 1, which is a compound of formula (I), which is selected from the group consisting of

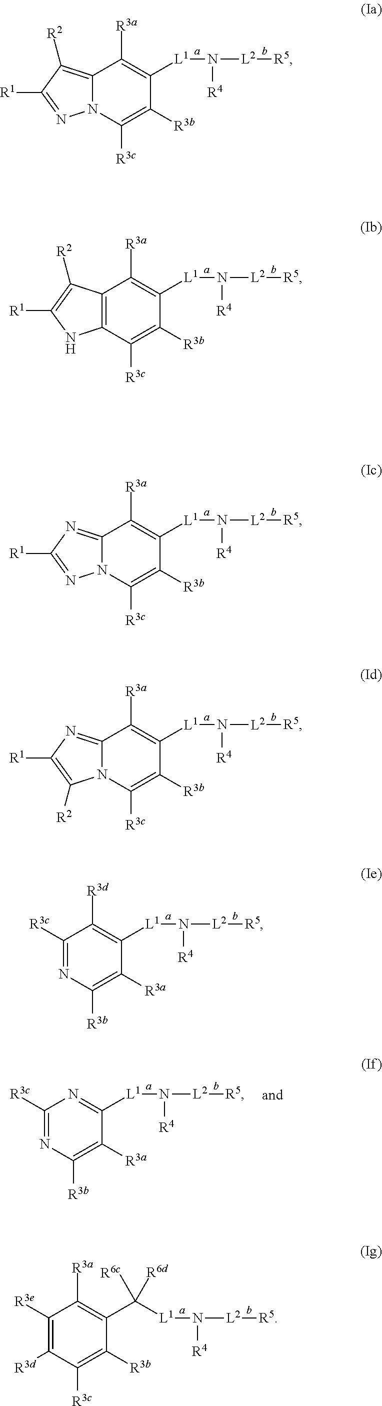

4. The compound of claim 1, wherein at least one of the following applies:
(a) R¹ is H;
(b) R² is selected from the group consisting of H, F, Br, CN, C(═O)OH, C(═O)NH₂, C(═O)NHMe, C(═O)NHOH, and C(═NH)NH(OH);
(c) at least one selected from R^{3a}, R^{3b}, R^{3c}, R^{3d}, and R^{3e} is H;
(d) at least two selected from R^{3a}, R^{3b}, R^{3c}, R^{3d}, and R^{3e} are H;
(e) at least three selected from R^{3a}, R^{3b}, R^{3c}, R^{3d}, and R^{3e} are H;
(f) at least four selected from R^{3a}, R^{3b}, R^{3c}, R^{3d}, and R^{3e} are H; and
(g) each of R^{3a}, R^{3b}, R^{3c}, R^{3d}, and R^{3e} are H.

5.-6. (canceled)

7. The compound of claim 1, wherein T is selected from the group consisting of:

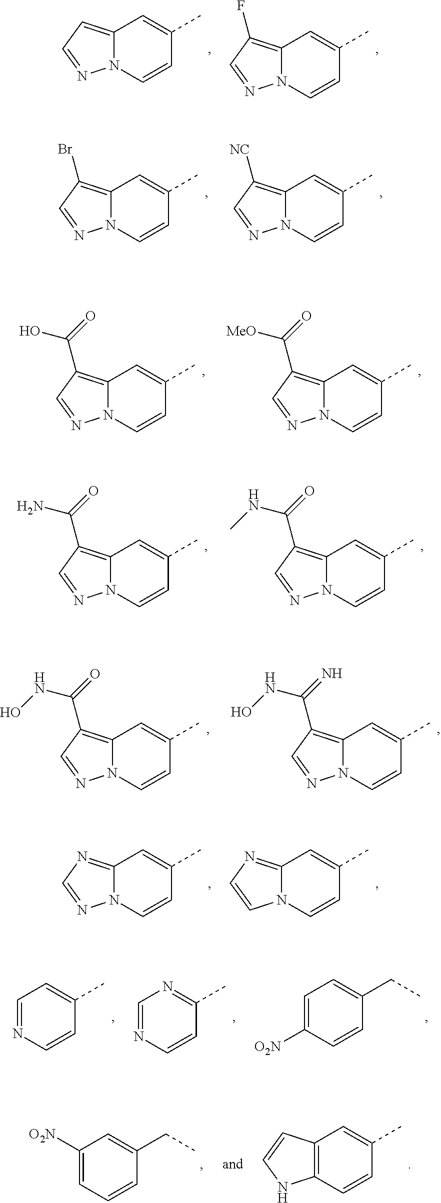

8. The compound of claim 1, wherein R⁴ is selected from the group consisting of H, methyl, isopropyl, 1-methylpropyl, 2-methylpropyl, 3-methylbutyl, cyclopropyl, cyclobutyl, cyclopentyl, cyclohexyl, cycloheptyl, (CH₂)₃N(CH₃)₂, C(═O)CH₃, phenyl, benzyl substituted with at least one C₁-C₆ alkyl; and —CH₂CH₂CH₂O(phenyl substituted with at least one halogen),
optionally wherein R⁴ is selected from the group consisting of

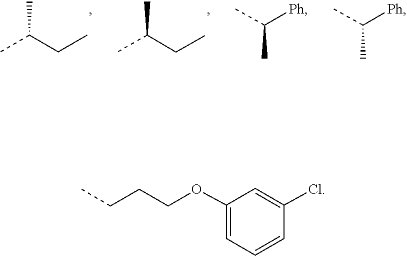

9. (canceled)

10. The compound of claim 1, wherein A¹ is selected from the group consisting of H; phenyl substituted with at least one substituent selected from the group consisting of C₁-C₆ alkyl, C₁-C₆ alkoxy, C₁-C₃ haloalkyl, C₁-C₃ haloalkoxy, C₃-C₈ cycloalkyl optionally substituted with a C₁-C₃ haloalkyl, phenoxy, halogen, and NO₂; phenyl fused with a C₂-C₆ heterocycloalkyl; and C₂-C₈ heteroaryl optionally substituted with C₁-C₆ alkyl or fused with a C₂-C₅ heteroaryl;
optionally wherein A¹ is selected from the group consisting of:

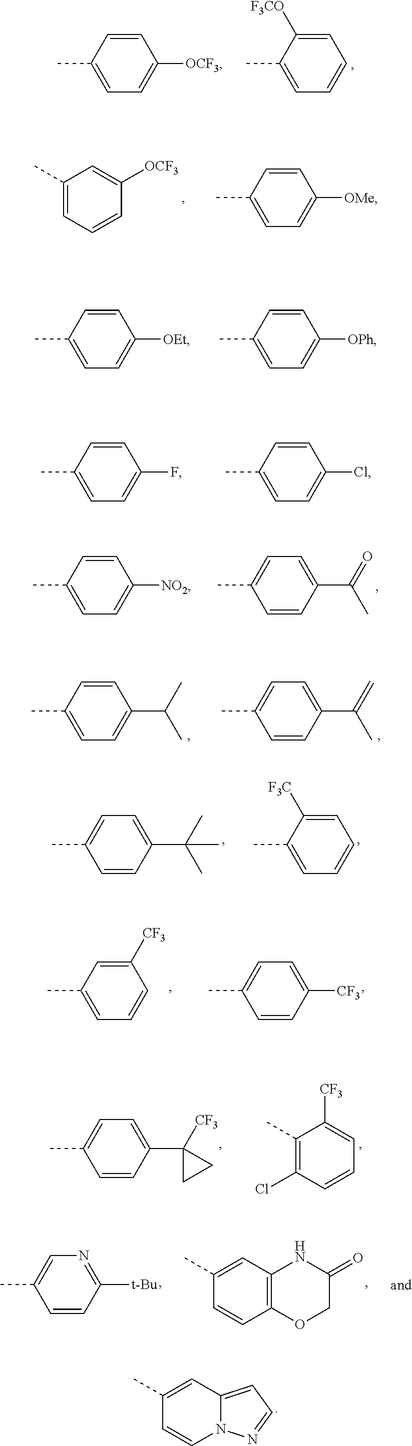

11. (canceled)

12. The compound of claim 1, wherein T least one of the following applies:
(a) L¹ is —CH₂—,
(b) L² is selected from the group consisting of a bond, —C(═O)—, —C(═S)—, S(═O)₂—, —CH₂—, 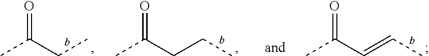
(c) X¹ is selected from the group consisting of a bond, O, and NR⁷, optionally wherein R⁷ is selected from the group consisting of H and Me.

13.-15. (canceled)

16. The compound of claim 1, wherein R⁵ is selected from the group consisting of:

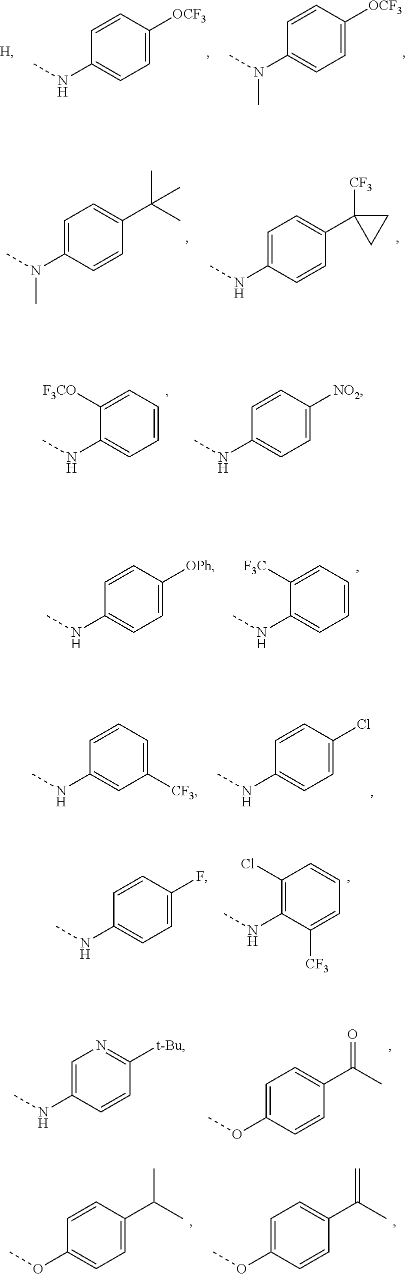

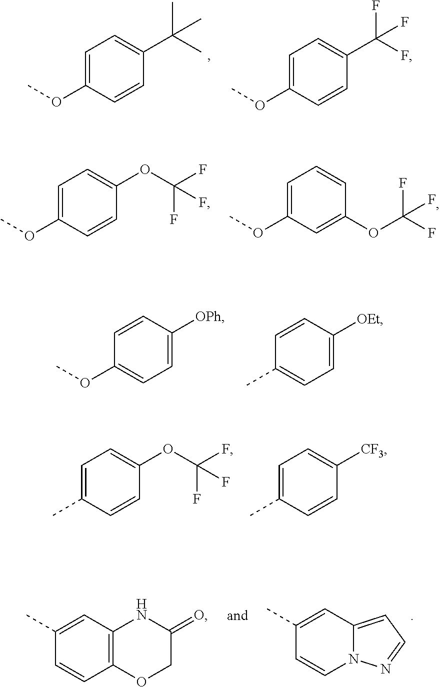

17. The compound of claim 1, which is selected from the group consisting of:
5-((1-isopropyl-3-(4-(trifluoromethoxy)phenyl)ureido)methyl)pyrazolo[1,5-a]pyridine-3-carboxylic acid;
1-cycloheptyl-1-(pyrazolo[1,5-a]pyridin-5-ylmethyl)-3-(4-(trifluoromethoxy)phenyl)urea;
1-cyclobutyl-1-(pyrazolo[1,5-a]pyridin-5-ylmethyl)-3-(2-(trifluoromethoxy)phenyl)urea;
1-isopropyl-1-(pyrazolo[1,5-a]pyridin-5-ylmethyl)-3-(4-(trifluoromethoxy)phenyl)urea;
1-cyclohexyl-1-(pyrazolo[1,5-a]pyridin-5-ylmethyl)-3-(4-(trifluoromethoxy)phenyl)urea;
1-cyclobutyl-3-(4-nitrophenyl)-1-(pyrazolo[1,5-a]pyridin-5-ylmethyl)urea;
1-phenyl-1-(pyrazolo[1,5-a]pyridin-5-ylmethyl)-3-(4-(trifluoromethoxy)phenyl)urea;
1-cyclopentyl-1-(pyrazolo[1,5-a]pyridin-5-ylmethyl)-3-(4-(trifluoromethoxy)phenyl)urea;
1-cyclopropyl-1-(pyrazolo[1,5-a]pyridin-5-ylmethyl)-3-(4-(trifluoromethoxy)phenyl)urea;
1-cyclobutyl-1-(pyrazolo[1,5-a]pyridin-5-ylmethyl)-3-(2-(trifluoromethyl)phenyl)urea;
1-cyclobutyl-1-(pyrazolo[1,5-a]pyridin-5-ylmethyl)-3-(3-(trifluoromethyl)phenyl)urea;
N-(3-(3-chlorophenoxy)propyl)-2-(4-isopropylphenoxy)-N-(pyrazolo[1,5-a]pyridin-5-ylmethyl)acetamide;
methyl 5-((cyclobutylamino)methyl)pyrazolo[1,5-a]pyridine-3-carboxylate;
3-(3-chlorophenoxy)-N-(pyrazolo[1,5-a]pyridin-5-ylmethyl)propan-1-amine;
N,N-bis(pyrazolo[1,5-a]pyridin-5-ylmethyl)cyclobutanamine;
1-(3-(3-chlorophenoxy)propyl)-3-(4-phenoxyphenyl)-1-(pyrazolo[1,5-a]pyridin-5-ylmethyl)urea;
1-(3-(3-chlorophenoxy)propyl)-1-(pyrazolo[1,5-a]pyridin-5-ylmethyl)-3-(4-(trifluoromethoxy)phenyl)urea;
3-(4-chlorophenyl)-1-cyclobutyl-1-(pyrazolo[1,5-a]pyridin-5-ylmethyl)urea;
1-cyclobutyl-3-(4-fluorophenyl)-1-(pyrazolo[1,5-a]pyridin-5-ylmethyl)urea;
5-((1-cyclobutyl-3-(4-(trifluoromethoxy)phenyl)ureido)methyl)-N-methylpyrazolo[1,5-a]pyridine-3-carboxamide;
5-((1-cyclobutyl-3-(4-(trifluoromethoxy)phenyl)ureido)methyl)pyrazolo[1,5-a]pyridine-3-carboxylic acid;
methyl 5-((1-cyclobutyl-3-(4-(trifluoromethoxy)phenyl)ureido)methyl)pyrazolo[1,5-a]pyridine-3-carboxylate;
1-cyclobutyl-3-(4-methoxyphenyl)-1-(pyrazolo[1,5-a]pyridin-5-ylmethyl)urea;
(R)-2-(4-ethoxyphenyl)-N-(1-phenylethyl)-N-(pyrazolo[1,5-a]pyridin-5-ylmethyl)acetamide;
(S)-2-(4-ethoxyphenyl)-N-(1-phenylethyl)-N-(pyrazolo[1,5-a]pyridin-5-ylmethyl)acetamide;
1-cyclobutyl-1-(pyrazolo[1,5-a]pyridin-5-ylmethyl)-3-(4-(trifluoromethoxy)phenyl)urea;
(S)-1-phenyl-N-(pyrazolo[1,5-a]pyridin-5-ylmethyl)ethan-1-amine;
1-((3-fluoropyrazolo[1,5-a]pyridin-5-yl)methyl)-1-isopropyl-3-(4-(trifluoromethoxy)phenyl)urea;
1-((3-bromopyrazolo[1,5-a]pyridin-5-yl)methyl)-1-isopropyl-3-(4-(trifluoromethoxy)phenyl)urea;
5-((1-cyclopropyl-3-(4-(trifluoro methoxy)phenyl) ureido)methyl)pyrazolo[1,5-a]pyridine-3-carboxylic acid;
5-((1-isopropyl-3-(4-(trifluoro methoxy)phenyl) ureido)methyl)pyrazolo[1,5-a]pyridine-3-carboxamide;
5-((1-cyclopropyl-3-(4-(trifluoro methoxy)phenyl) ureido)methyl)pyrazolo[1,5-a]pyridine-3-carboxamide;
5-((1-isopropyl-3-methyl-3-(4-(trifluoro methoxy) phenyl)ureido)methyl)pyrazolo[1,5-a]pyridine-3-carboxylic acid;
N-hydroxy-5-((1-isopropyl-3-methyl-3-(4-(trifluoro methoxy)phenyl)ureido)methyl)pyrazolo[1,5-a]pyridine-3-carboxamide;
1-((3-cyanopyrazolo[1,5-a]pyridin-5-yl)methyl)-1-isopropyl-3-(4-(trifluoromethoxy)phenyl)urea;
5-((1-isobutyl-3-(4-(trifluoromethoxy)phenyl)ureido)methyl)pyrazolo[1,5-a]pyridine-3-carboxylic acid;
5-((1-(sec-butyl)-3-(4-(trifluoromethoxy)phenyl) ureido)methyl)pyrazolo[1,5-a]pyridine-3-carboxylic acid;
N-hydroxy-5-((1-isopropyl-3-(4-(trifluoromethoxy) phenyl)ureido)methyl)pyrazolo[1,5-a]pyridine-3-carboximidamide;
5-((1-methyl-3-(4-(trifluoromethoxy)phenyl)thioureido)methyl)pyrazolo[1,5-a]pyridine-3-carboxamide;
5-((1-isopropyl-3-(4-(trifluoromethoxy)phenyl)thioureido)methyl)pyrazolo[1,5-a]pyridine-3-carboxamide;
5-((2-(4-isopropylphenoxy)-N-methylacetamido)methyl)pyrazolo[1,5-a]pyridine-3-carboxamide;
5-((N-methyl-3-(4-(trifluoromethoxy)phenyl)propanamido)methyl)pyrazolo[1,5-a]pyridine-3-carboxamide;
5-((1-methyl-3-(4-(trifluoromethoxy)phenyl)ureido)methyl)pyrazolo[1,5-a]pyridine-3-carboxamide;
5-((N-isopropyl-2-(4-isopropylphenoxy)acetamido)methyl)pyrazolo[1,5-a]pyridine-3-carboxamide;
5-((N-(3-(dimethylamino)propyl)-2-(4-isopropylphenoxy)acetamido)methyl)pyrazolo[1,5-a]pyridine-3-carboxamide;
5-((2-(4-isopropylphenoxy)acetamido)methyl)pyrazolo[1,5-a]pyridine-3-carboxamide;
5-((3-(4-(trifluoromethoxy)phenyl)ureido)methyl)pyrazolo[1,5-a]pyridine-3-carboxamide;
5-((3-(4-(trifluoromethoxy)phenyl)thioureido)methyl)pyrazolo[1,5-a]pyridine-3-carboxamide;
5-((N-methyl-2-(4-(trifluoromethoxy)phenoxy)acetamido)methyl)pyrazolo[1,5-a]pyridine-3-carboxamide;
5-((N-isopropyl-2-(4-(trifluoromethoxy)phenoxy)acetamido)methyl)pyrazolo[1,5-a]pyridine-3-carboxamide;
5-((1-(sec-butyl)-3-(4-(trifluoromethoxy)phenyl)ureido)methyl)pyrazolo[1,5-a]pyridine-3-carboxamide;
(R)-5-((1-(sec-butyl)-3-(4-(trifluoromethoxy)phenyl)ureido)methyl)pyrazolo[1,5-a]pyridine-3-carboxamide;
(S)-5-((1-(sec-butyl)-3-(4-(trifluoromethoxy)phenyl)ureido)methyl)pyrazolo[1,5-a]pyridine-3-carboxamide;
5-((N-methyl-2-(4-(trifluoromethyl)phenoxy)acetamido)methyl)pyrazolo[1,5-a]pyridine-3-carboxamide;
5-((N-methyl-2-(3-(trifluoromethoxy)phenoxy)acetamido)methyl)pyrazolo[1,5-a]pyridine-3-carboxamide;
5-((2-(4-(tert-butyl)phenoxy)-N-methylacetamido)methyl)pyrazolo[1,5-a]pyridine-3-carboxamide;
5-((2-(4-(tert-butyl)phenoxy)-N-isopropylacetamido)methyl)pyrazolo[1,5-a]pyridine-3-carboxamide;
5-((2-((4-(tert-butyl)phenyl)(methyl)amino)-N-methylacetamido)methyl)pyrazolo[1,5-a]pyridine-3-carboxamide;
5-((N-isopropyl-2-((4-(trifluoromethoxy)phenyl)amino)acetamido)methyl)pyrazolo[1,5-a]pyridine-3-carboxamide;
5-((N-isopropyl-2-(4-(prop-1-en-2-yl)phenoxy)acetamido)methyl)pyrazolo[1,5-a]pyridine-3-carboxamide;
5-((2-(4-acetylphenoxy)-N-isopropylacetamido)methyl)pyrazolo[1,5-a]pyridine-3-carboxamide;
5-((2-(4-acetylphenoxy)-N-methylacetamido)methyl)pyrazolo[1,5-a]pyridine-3-carboxamide;
5-((N-methyl-2-((4-(trifluoromethoxy)phenyl)amino)acetamido)methyl)pyrazolo[1,5-a]pyridine-3-carboxamide;
2-(3-(2-chloro-6-(trifluoromethyl)phenyl)ureido)-N,N-dimethyl-3-(3-nitrophenyl)propenamide;
(S)-2-(3-(2-chloro-6-(trifluoromethyl)phenyl)ureido)-N,N-dimethyl-3-(3-nitrophenyl)propenamide;
(R)-2-(3-(2-chloro-6-(trifluoromethyl)phenyl)ureido)-N,N-dimethyl-3-(3-nitrophenyl)propenamide;
N-methyl-3-(4-nitrophenyl)-2-(3-(4-phenoxyphenyl)ureido)propenamide;
(R)-N-methyl-3-(4-nitrophenyl)-2-(3-(4-phenoxyphenyl)ureido)propenamide;
(S)-N-methyl-3-(4-nitrophenyl)-2-(3-(4-phenoxyphenyl)ureido)propenamide;
N-((1H-indol-5-yl)methyl)-N-isopropyl-2-(4-(trifluoromethoxy)phenoxy)acetamide;
1-((1H-indol-5-yl)methyl)-1-isopropyl-3-(4-(trifluoromethoxy)phenyl)urea;
2-((6-(tert-butyl)pyridin-3-yl)amino)-N-isopropyl-N-(pyrazolo[1,5-a]pyridin-5-ylmethyl)acetamide;
N-isopropyl-N-(pyrazolo[1,5-a]pyridin-5-ylmethyl)-2-((4-(1-(trifluoromethyl)cyclopropyl)phenyl)amino)acetamide;
5-((methyl(N-(4-(trifluoromethoxy)phenyl)sulfamoyl)amino)methyl)pyrazolo[1,5-a]pyridine-3-carboxamide;
1-([1,2,4]triazolo[1,5-a]pyridin-7-ylmethyl)-1-cyclopropyl-3-(4-(trifluoromethoxy)phenyl)urea;
1-cyclopropyl-1-(pyridin-4-ylmethyl)-3-(4-(trifluoromethoxy)phenyl)urea;
1-cyclopropyl-1-(pyrimidin-4-ylmethyl)-3-(4-(trifluoromethoxy)phenyl)urea;
1-cyclopropyl-1-(imidazo[1,2-a]pyridin-7-ylmethyl)-3-(4-(trifluoromethoxy)phenyl)urea;
5-((N-(3-(3-chlorophenoxy)propyl)-2-(4-isopropylphenoxy)acetamido)methyl)pyrazolo[1,5-a]pyridine-3-carboxamide;
5-((N-(4-(trifluoromethoxy)phenyl)acetamido)methyl)pyrazolo[1,5-a]pyridine-3-carboxamide;
5-(((N-isopropyl-4-(trifluoromethyl)phenyl)sulfonamido)methyl)pyrazolo[1,5-a]pyridine-3-carboxamide;
5-(((N-isopropyl-3-oxo-3,4-dihydro-2H-benzo[b][1,4]oxazine)-6-sulfonamido)methyl)pyrazolo[1,5-a]pyridine-3-carboxamide;
5-(((N-methyl-4-(trifluoromethoxy)phenyl)sulfonamido)methyl)pyrazolo[1,5-a]pyridine-3-carboxamide;
5-(((N-isopropyl-4-(trifluoromethoxy)phenyl)sulfonamido)methyl)pyrazolo[1,5-a]pyridine-3-carboxamide;
5-((N-isopropyl-3-(4-methoxyphenyl)acrylamido)methyl)pyrazolo[1,5-a]pyridine-3-carboxamide;
(E)-5-((N-isopropyl-3-(4-methoxyphenyl)acrylamido)methyl)pyrazolo[1,5-a]pyridine-3-carboxamide;
(Z)-5-((N-isopropyl-3-(4-methoxyphenyl)acrylamido)methyl)pyrazolo[1,5-a]pyridine-3-carboxamide;
5-((N-isopropyl-3-(4-(trifluoromethyl)phenyl)acrylamido)methyl)pyrazolo[1,5-a]pyridine-3-carboxamide;
(E)-5-((N-isopropyl-3-(4-(trifluoromethyl)phenyl)acrylamido)methyl)pyrazolo[1,5-a]pyridine-3-carboxamide; and
(Z)-5-((N-isopropyl-3-(4-(trifluoromethyl)phenyl)acrylamido)methyl)pyrazolo[1,5-a]pyridine-3-carboxamide.

18. The compound of claim 1, which is a compound of formula (II).

19. The compound of claim 18, wherein at least one of the following applies:
(a) R⁸ is selected from the group consisting of isopropyl and phenyl;
(b) R^{9a}, R^{9b}, R^{9c}, and R^{9d} are each independently selected from the group consisting of H, tert-butyl, and trifluoromethyl;
(c) R¹⁰ is selected from the group consisting of tert-butyl, 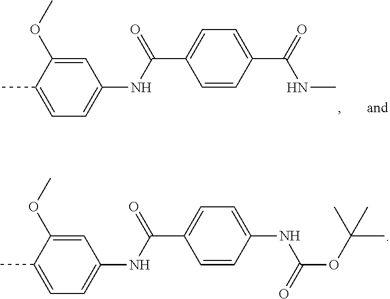
(d) R^{11a}, R^{11b}, R^{11c}, R^{11d}, R^{11e}, R^{11f}, R^{11g}, and R^{11h} are each independently selected from the group consisting of H and methoxy;
(e) R^{12a}, R^{12b}, and R^{12c} are each independently selected from the group consisting of H, methyl, and C(═O)O(C(CH₃)₃).

20. (canceled)

21. The compound of claim 18, wherein at least one of the following applies:
(a) A² is selected from the group consisting of 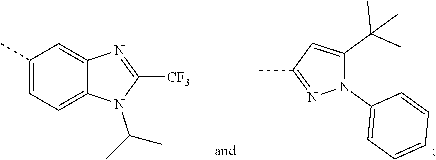
(b) X² is selected from the group consisting of —O— and —NH—.

22. The compound of claim 18, wherein one of the following applies:
(a) Z¹ is N and at least one of Z², Z³, and Z⁴ is CH;
(b) Z¹ is N and at least two of Z², Z³, and Z⁴ are CH;
(c) Z¹ is N and each of Z², Z³, and Z⁴ are CH;
(d) Z² is N and at least one of Z¹, Z³, and Z⁴ is CH;
(e) Z² is N and at least two of Z¹, Z³, and Z⁴ are CH; or
(f) Z² is N and each of Z¹, Z³, and Z⁴ are CH.

23.-26. (canceled)

27. The compound of claim 1, which is selected from the group consisting of:
N¹-(4-(5-(5-(5-(tert-butyl)-1-phenyl-1H-pyrazol-3-yl)-1,2,4-oxadiazol-3-yl)picolinamido)-3-methoxyphenyl)-N⁴-methylterephthalamide;
N¹-(4-(5-(5-(1-isopropyl-2-(trifluoromethyl)-1H-benzo[d]imidazol-5-yl)-1,2,4-oxadiazol-3-yl)picolinamido)-3-methoxyphenyl)-N⁴-methylterephthalamide;
tert-butyl 5-(5-(5-(tert-butyl)-1-phenyl-1H-pyrazol-3-yl)-1,2,4-oxadiazol-3-yl)picolinate;
tert-butyl 5-(5-(1-isopropyl-2-(trifluoromethyl)-1H-benzo[d]imidazol-5-yl)-1,2,4-oxadiazol-3-yl)picolinate;
(Z)-N¹-(4-(5-(N′-((5-(tert-butyl)-1-phenyl-1H-pyrazole-3-carbonyl)oxy)carbamimidoyl)picolinamido)-3-methoxyphenyl)-N⁴-methylterephthalamide; and
tert-butyl (4-(5-(5-(1-isopropyl-2-(trifluoromethyl)-1H-benzo[d]imidazol-5-yl)-1,2,4-oxadiazol-3-yl)picolinamido)-3-methoxyphenyl)carbamate.

28. The compound of claim 1, wherein the compound is a compound of formula (III).

29. The compound of claim 28, wherein the compound of formula (III) is selected from the group consisting of:

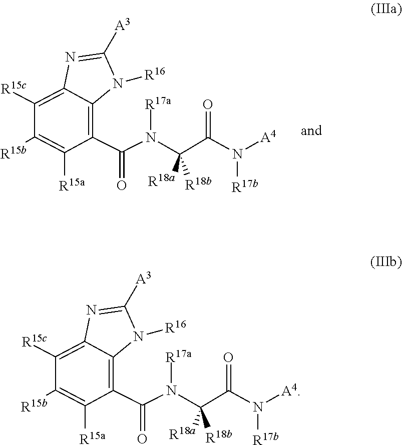

30. The compound of claim 28, wherein at least one of the following applies:
(a) at least one of R^{15a}, R^{15b}, and R^{15c} is H;
(b) at least two of R^{15a}, R^{15b}, and R^{15c} are H;
(c) each of R^{15a}, R^{15b}, and R^{15c} are H;
(d) R¹⁶ is selected from the group consisting of benzyl and 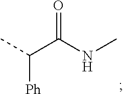
(e) R^{17a} and R^{17b} are each independently H; and
(f) R^{18a} and R^{18b} are each independently selected from the group consisting of H and Me.

31.-33. (canceled)

34. The compound of claim 28, wherein at least one of the following applies:
(a) A³ is selected from the group consisting of 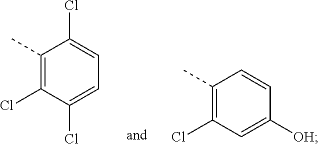
(b) A⁴ is

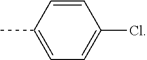

35. (canceled)

36. The compound of claim 1, which is selected from the group consisting of:
N-((S)-1-((4-chlorophenyl)amino)-1-oxopropan-2-yl)-1-(2-(methylamino)-2-oxo-1-phenylethyl)-2-(2,3,6-trichlorophenyl)-1H-benzo[d]imidazole-7-carboxamide;
(S)-1-benzyl-2-(2-chloro-4-hydroxyphenyl)-N-(1-((4-chlorophenyl)amino)-1-oxopropan-2-yl)-1H-benzo[d]imidazole-7-carboxamide; and
(S)-1-benzyl-N-(1-((4-chlorophenyl)amino)-1-oxopropan-2-yl)-2-(2,3,6-trichlorophenyl)-1H-benzo[d]imidazole-7-carboxamide.

37. A pharmaceutical composition comprising the compound of claim 1 and a pharmaceutically acceptable carrier.

38. A method of treating, preventing, or ameliorating at least one of endometriosis, cancer, and pain in a subject in need thereof,
the method comprising administering to the subject at least one Neurotrophic Receptor Tyrosine Kinase (NTRK) inhibitor or a pharmaceutical composition comprising the NTRK inhibitor and a pharmaceutically acceptable carrier;
optionally wherein the subject is a mammal, which is optionally a human.

39. The method of claim 38, wherein the subject is female and suffers from endometriosis, and wherein at least one selected from the group consisting of chronic pelvic pain, inflammation, and infertility is treated, prevented, or ameliorated in the subject.

40.-42. (canceled)

43. The method of claim 38, wherein at least one selected from the group consisting of neurotrophic receptor tyrosine kinase 1 (NTRK1), neurotrophic receptor tyrosine kinase 2 (NTRK2), and neurotrophic receptor tyrosine kinase 3 (NTRK3), is overexpressed in the subject.

44. The method of claim 38, wherein at least one selected from the group consisting of NTRK1, NTRK2, and NTRK3 is inhibited in the subject.

45. The method of claim 38, wherein a fusion gene comprising at least one selected from the group consisting of NTRK1, NTRK2, and NTRK3 is overexpressed in the subject, optionally wherein the fusion gene product is inhibited in the subject.

46. (canceled)

47. The method of claim 38, wherein the NTRK inhibitor comprises the compound of claim 1 or a pharmaceutical composition thereof.

48. The method of claim 38, wherein the NTRK inhibitor is a NTRK2 inhibitor,
optionally wherein the NTRK2 inhibitor selectively inhibits NTRK2 over at least one of NTRK1 and NTRK3,
optionally wherein the at least one NTRK2 inhibitor is selected from the group consisting of staurosporine, larotrectinib, entrectinib, selitrectinib, reprotrectinib, and PF-06273340.

49.-52. (canceled)
